(19) United States
(12) Patent Application Publication
(10) Pub. No.: US 2026/0224659 A1
(43) Pub. Date: Aug. 6, 2026

(54) PEPTIDES FOR TREATMENT OF SKIN DISEASE

(71) Applicant: In2Cure AB, Lund (SE)

(72) Inventors: Helene Hartman, Lund (SE); Artur Schmidtchen, Lund (SE)

(21) Appl. No.: 19/151,427

(22) PCT Filed: Jan. 30, 2024
(86) PCT No.: PCT/EP2024/052168

§ 371 Date: Jul. 28, 2025

Related U.S. Application Data
(60) Provisional application No. 63/441,949, filed on Jan. 30, 2023.

(30) Foreign Application Priority Data
Jan. 30, 2023 (JP) 2023-011773

(51) Int. Cl. A61K 38/16; A61K 38/10; A61P 17/02

(52) U.S. Cl. CPC A61K 38/16; A61K 38/10; A61P 17/02

(57) ABSTRACT

The present invention relates to peptides for treatment of skin diseases and skin conditions. In particular, the present invention relates to peptide for treatment of wounds and/or blisters in patients suffering from a skin disease or skin condition selected from the group consisting of blistering disease, a disease or condition characterized by excessive wound exudate, a disease or condition characterised by a dysregulated skin microbiome and scars.

### TECHNICAL FIELD

The present invention relates to peptides for treatment of skin diseases and skin conditions. In particular, the present invention relates to peptide for treatment of wounds and/or blisters in patients suffering from a skin disease or skin condition selected from the group consisting of blistering disease, a disease or condition characterized by excessive wound exudate, a disease or condition characterised by a dysregulated skin microbiome and scars.

### BACKGROUND

In normal conditions, wounds heal in a sequenced and timely manner, characterized by four major phases (haemostasis, inflammation, proliferation, and remodelling). This is a complex process involving chemokines, growth factors, cytokines, proteases and antiproteases, and multiple cell types that all work in a controlled manner during the healing process. If this timely process is interrupted at various stages and by different factors, a dysfunctional inflammatory phase occurs, which may be associated with disruption in wound healing. Toll-like receptors (TLR), crucial for normal wound repair, can become over-activated and induce local dysfunctional inflammation which may delay wound healing. Some wounds, for example epidermolysis Bullosa (EB) wounds, are often complicated by bacterial infection or hyper-colonization, where bacteria and their proinflammatory products (also denoted PAMPs), such as endotoxins (lipopolysaccharide/LPS) from Gram-negative bacteria, lipoteichoic acid (LTA) and peptidoglycan (PGN) from Gram-positive bacteria, activate the TLR signalling and induce an excessive non-controlled inflammation with high tumour necrosis factor-alfa (TNF-α), interleukin (IL)-6, and IL-1β levels yielding a high level of proteolysis, which may lead to delayed healing. PAMPs may lead to TLR-mediated nuclear factor (NF) KB activation, which in turn may induce local cytokine release and secondary tissue damage.

### Epidermolysis Bullosa (EB)

EB comprises a group of genetic disorders manifested by mechanically induced blistering and fragility of the skin and other stratified epithelia. Four types are recognized depending on the level of blistering in the extended epidermal basement membrane zone, EB simplex, junction EB, dystrophic EB with the subgroups dominant dystrophic EB and recessive dystrophic EB (RDEB), and Kindler EB. EB is linked to a high wound burden and wounds have thus served as a frequently used endpoint for clinical trials.

Inflammation is notable in EB skin and promotes pain and itch (Papanikolaou et al., 2021). There are indications that a dysregulated inflammatory response could be involved in delays in wound healing and promoting fibrosis in recessive dystrophic epidermolysis bullosa (RDEB) (Bernasconi et al., 2021). Another factor that may be involved in impairment of healing and promoting chronicity of wounds are bacterial inflammation, which occurs in wounded skin of all EB types (van der Kooi-Pol et al., 2012). Wound infections may promote wound-induced malignant transformation of keratinocytes and development of cutaneous squamous cell carcinomas, which can occur in all EB types and is the major cause of death in severe RDEB (Hoste et al., 2015; Robertson et al., 2021). Apart from being painful and debilitating, the high wound burden in EB constitutes a considerable economic burden. In RDEB, the annual cost for wound dressings has in the US been reported to range from $4,000 to $245,000 (Tang et al., 2021).

### Blisters

Blisters occur in all types of EB following friction and relatively mild trauma. They can be present anywhere on the skin and the mucous membranes. The location of a particular blister may be EB type specific. For example, in epidermolysis bullosa simplex (EBS) blistering will mainly occur on the hands and feet. In other forms of EB, such as dystrophic EB, blisters will occur on the areas subject to the most trauma, such as the bony prominences. The blisters can occur alone or in clusters depending on the initial degree of trauma and they may be filled with serous or blood-stained fluid. Blisters are not self-limiting and will fill with serous fluid and rapidly expand if left intact. Blister formation in EB is the first sequence in wound generation. The blisters contain sterile exudate (Cutting, 2003; Adderley, 2008). Exudate formation in EB is dependent on epidermal loss, combined with leakage through capillaries underneath. This feature is common for EB wounds and other acute epidermal wounds.

### Exudate

Many factors can influence the production of exudate. In some cases, infection may increase exudate production. However, acute wounds, such as EB wounds, that are non-infected also produce exudate.

Exudation is a problem in wound care. Some wounds, for example chronic wounds or EB wounds, often produce an excessive amount of exudate. If this is not controlled, the wound dressing becomes saturated and moisture may leak to the skin surrounding the wound (i.e. the periwound skin), which can cause maceration and excoriation. Macerated skin breaks down easily, which can result in an increased wound size. Moreover, blister formation may progress, increasing blister and wound size (Pillay and Clapham, 2018).

Exudate can have a considerable impact on an EB patient's quality of life. Excess exudate can lead to problems of discomfort, pain, malodour, leaking (e.g. leaking wound dressing). Wound exudate can therefore have a high impact on the life of an EB patient, for example by making it necessary to change dressings very frequently. The pain and fatigue, as well as the bulky dressings, can make it difficult for EB patients to bathe and change their clothes (Pillay and Clapham, 2018).

Inappropriate wound management and selection of wound dressing can also contribute to the problem; for example, applying a dressing designed to absorb high levels of exudate to a low to moderately exuding wound may cause a ‘drawing’ pain, or may adhere to the wound bed causing pain and trauma on removal as delicate healing tissue is damaged.

If the dressing product or therapy used does not manage the exudate and strike-through (leakage of exudate through a dressing) occurs, this can lead to an increased risk of infection. Furthermore, when dressings are being removed too often, owing to an increased frequency of dressing change, this can result in damage to the surrounding skin through epidermal stripping.

### Skin/Wound Microbiome

The skin is an ecosystem with multiple niches, each featuring unique physiological conditions and thus hosting different bacterial populations. A dysfunctional skin microbiome has been implicated in the pathogenesis of EB (Levin et al., 2021), hereunder dystrophic epidermolysis bullosa (DEB) (Bar et al., 2020).

### Other Wounds

The above-mentioned complications are present also in other wounds. In particular, inflammation, infection, wound leakage, microbial dysbiosis and scarring are common complications in patients suffering from other types of blistering diseases than EB, as well as in patients suffering from burn wounds, chronic skin ulcers, infected acute wounds, pressure ulcers and chronic wounds such as venous ulcers or diabetic leg ulcers.

### Conclusion

Although advances are being made in general wound care, current treatment options for EB are limited to keeping wounds clean through e.g., disinfectants (Octenisept, Prontosan etc.), antiseptic baths and silver-containing creams but these approaches come with significantly challenges and are inadequately effective (Pillay and Clapham, 2018; Pope et al., 2012; Prodinger et al., 2019). As antimicrobial resistance increases, common antibiotics and some antiseptics are becoming even less effective also in treatment of wound infections. Furthermore, many of the existing treatments, especially silver-containing creams, are harmful for the environment as well as toxic to the patient if used long-term. Therefore, there is a great need for new and cost-effective treatments that will improve healing in patients with EB. In relation to leakage in absence of infection, no treatments currently exist that can target excessive wound leakage in EB wounds.

Thus, in summary, there is a great need for treatment that can specifically target wound leakage, while preserving the commensal microbiome, as well as reduce and normalize the dysfunctional microbiome, including excessive levels of pathogenic S. aureus. In other words, there is great need for treatment that simultaneously addresses wound leakage, microbial dysbiosis and wound healing and scarring in EB wounds as well as in other wounds.

### SUMMARY

Surprisingly, the inventors have discovered that thrombin-derived C-terminal peptides can be used in the treatment of various skin conditions and skin diseases. In particular, the inventors have discovered that said peptides can be used for treating a disease or condition characterized by blistering, a disease or condition characterized by excessive wound exudate, a disease or condition characterised by a dysregulated skin microbiome and scars.

It has previously been shown that thrombin derived C-terminal peptides can be used to treat inflammation and infection. The present invention demonstrates that thrombin derived peptides are effective in treatment of EB. The invention further shows that thrombin derived peptides are useful for treatment of several symptoms which may be associated with certain skin conditions and skin diseases. Such symptoms include excessive wound exudate, dysregulated skin microbiome and scars.

Previous findings suggest the existence of a unique EB-associated skin microbiome dysbiosis (Bar et al., 2020; Levin et al., 2021). A similar dysbiosis has been found to be associated with other complex wounds, including burns and chronic skin ulcers (such as venous or diabetic) (Byrd, 2018). The inventors have discovered that thrombin derived C-terminal peptides may alter such dysbiosis, facilitating wound healing in patients with EB and other complex wounds.

All of the above-mentioned symptoms may be treated with thrombin derived peptides in association with any skin disease or skin condition associated with one or more, or even all, of said symptoms. As the symptoms are different at their core, they have traditionally been addressed by separate treatment approaches. However, as the inventors have discovered, the peptides of the current invention provides in a single treatment approach a curing, ameliorating or alleviating effect on said symptoms, such that the individual in need of treatment must not use several different medicaments or aids.

Thus, the present invention relates to:
a method of treatment of a skin disease or skin condition selected from the group consisting of a disease or condition characterized by blistering, a disease or condition characterized by excessive wound exudate, a disease or condition characterised by a dysregulated skin microbiome and scars,
a method of treatment of a skin disease or skin condition in an individual in need thereof, wherein the skin disease or skin condition is a wound or a disease or condition characterized by blistering, and wherein the treatment reduces the amount of fluid that exudes from one or more wound or blister,
a method of reducing the amount of fluid that exudes from one or more wound or blister in an individual in need thereof,
and
a method for promoting a healthy skin microbiome in an individual in need thereof,
said methods comprising administering a compound comprising a peptide comprising or consisting of the amino acid sequence

X₁-X₂-X₃-X₄-X₅-X₆-W-X₈-X₉-X₁₀,

wherein
   X_{4, 6, 9} is any standard amino acid,
   X₁ is I, L or V,
   X₂ is any standard amino acid except C,
   X₃ is A, E, Q, R or Y,
   X₅ is any standard amino acid except R,
   X₈ is I or L,
   X₁₀ is any standard amino acid except H,
wherein said peptide has a length of from 10 to 100 amino acid residues

The invention also relates to compositions comprising said compound comprising a peptide for use in any of the aforementioned methods.

### DESCRIPTION OF DRAWINGS

FIG. 1. Schematic illustration of clinical study. Schematic representation of a randomised, double-blinded FIH study.

FIG. 2. Heat map of bacterial development. The heatmaps show the bacterial count in swabs and dressing fluids (DF).

FIG. 3. Graph showing bacterial development. The boxplots show the bacterial count in swabs and dressing fluids (DF).

FIG. 4. The level of bacteria in the controls was normalized, and the bacterial level after TCP-25 treatment was expressed relative the control. The results showed that TCP-25 reduced the bacterial relative the adjacent control at all three dose levels.

FIG. 5. Illustration of replica results and definition of recorded areas. A Whatman paper is used to transfer bacteria from the wound and surroundings to an agar plate.

FIG. 6A-C. A replica method provides spatial data of the bacteria in the wounds. Grading of 1-4 represents bacterial density. TCP-25 gel reduced bacterial levels in the wound, and the periwound areas. The bacteria in the surrounding skin adjacent to the dressing remained unaffected.

FIG. 7. Bacteria was identified from the wound surface and the dressing at day 3 and 8 by Maldi TOF. The results showed that there was a development of a microbiome with usually 1-3 types present. Commensal bacteria were dominating, and it was observed that there was a correspondence between the types found at the wound surface and in the dressing. TCP-25 treatment did not change the overall composition of the commensal microbiome.

FIG. 8. TCP-25 treatment reduced bacterial levels particularly at the wound surface, and controlled excessive levels of S. aureus. A similar observation was made for wounds colonized with the species Bacillus.

FIG. 9. Wound exudation was determined by analysis of total protein content in the dressing fluid. The results showed that the bacterial levels did not correlate to protein content at day 5 and 8.

FIG. 10. TCP-25 treatment reduced exudation particularly at day 5 and 8 in a dose dependent manner.

FIG. 11. The difference in the levels of exudation was calculated relative the adjacent control wound. 2.6 and 8.6 mg/ml TCP-25 reduced protein content, and notably, 8.6 mg/ml TCP-25 showed significant reductions, with only 20-30% exudation relative the control.

FIG. 12. Illustration of reduced exudation at day 5 in wounds treated by TCP-25. Dressings with placebo gel show higher exudation.

FIG. 13. In a separate experiment performed as above, scarring and pigmentation was analysed. The results showed that TCP-25 reduced pigmentation at day 11.

FIG. 14. A S. aureus contaminated suture was implanted subcutaneously into mice. TCP-25 coated suture showed no fibrotic scar.

FIG. 15. Skin tissue of Lamc2jeb mice showing therapeutic effects of topical TCP-25 gel treatment. (A) Skin swab bacterial count and (B) dressing bacterial count. After TCP-25 treatment, a reduction in CFUs were noticed in swab and dressing samples. (C) Representative H&E stained photographs show epidermis and dermis. An immature epidermis and dermis with poor architecture is noticed in control gel treated area. However, TCP-25 gel treated skin showed comparatively mature epidermis with improved dermal architecture. Bar-chart shows skin histology score. An improved skin histology score was observed in the skin of TCP-25 gel treated mice.

FIG. 16. Skin wound tissue of Lamc2jeb mice showing therapeutic effects of topical TCP-25 gel treatment. (A) Wound swab and dressing bacterial count showing a reduction in CFUs after TCP-25 gel treatment. (B) Bar charts showing wound fluid cytokine measurements. A decrease in wound proinflammatory cytokines was observed after treatment with TCP-25 gel. (C) Representative H&E stained photographs show epidermis and dermis. An immature epidermis and dermis with poor architecture is noticed in control gel treated area. However, TCP-25 gel treated skin showed comparatively mature epidermis with improved dermal architecture. (D) Bar-chart shows wound histology score. An improved wound histology score was observed in the of TCP-25 gel treated mice.

FIG. 17. Skin wound tissue of Lamc2jeb mice showing therapeutic effects of topical TCP-25 gel treatment. (A) Wound swab and dressing bacterial count showing a reduction in CFUs after TCP-25 gel treatment. (B) Bacterial bioluminescence intensity visualized using IVIS bioimaging. Representative light emission intensity heat-map overlays are shown for 1, 3 and 6 h time point. Bar chart shows bioluminescence intensity emitted by the bacteria. A reduction in bacterial bioluminescence was observed after treatment with TCP-25 gel. (C) Wound healing rate showing an increase in wound healing after treatment with TCP-25.

FIG. 18. There is a statistically significant reduction in open wound area between baseline and last visit Day 29 in patients treated with TCP-25, both for the primary wounds and the larger secondary wounds. Whereas the reference wounds, matching the primary wounds, which were treated with standard of care (SOC) have a reduction in open wound area not reaching statistical significance. Paired t-test (2-tailed) with N=4 (patients with DEB). Mean and SD is shown in graph.

FIG. 19. Effects of TCP-25 on neutrophil-related proteins. (A) Median levels of the neutrophil proteins NE, MPO and HBP and (B) median levels of the neutrophil chemotactic factor IL8 in dressing fluid samples by ELISA. In all graphs, the median is represented by a horizontal line, interquartile range by a box, and range by whiskers. Left (L) and right (R) wounds from each patient were plotted separately and treated as individual biological replicates (n=16 wounds per group).

FIG. 20. TCP-25 treatment reduces wound leakage in DEB wounds. Leakage (exudation score) was reduced after treatment with TCP-25 gel at day 29 relative to day 8. (A) Average wound leakage of all primary (i.e “simple”) TCP-25 treated wounds (n=3) at day 8 and 29 and for reference SOC wounds (n=3) (left bar graph) and average wound leakage of secondary (i.e. “complex”) TCP-25 treated wounds (n=3) at day 8 and day 29 compared to additional non-TCP treated wounds of similar size (n=2) (right bar graph). FIG. 20B illustrates wound leakage of primary, secondary and reference SOC wounds for one of the treated patients.

FIG. 21. Mean (SD) reduction of open wound area (cm²) from Baseline (Day 1), at Day 29 for the matching wound pair: the primary wound treated with TCP-25 compared to the reference wound treated with standard of care (SOC). (n=4 patients with dystrophic EB).

FIG. 22. Levels of IL-1β, TNF-α, and IL-6 in the secondary TCP-treated wounds observed after treatment for 15 and 29 days compared with non-TCP25-treated wounds of similar size.

### DETAILED DESCRIPTION

### Definitions

In this specification, unless otherwise specified, “a” or “an” means “one or more”.

The term “amino acid”, as used herein, includes the twenty standard amino acids and their corresponding stereoisomers in the ‘D’ form (as compared to the natural ‘L’ form), omega-amino acids other naturally-occurring amino acids, unconventional amino acids (e.g., α,α-disubstituted amino acids, N-alkyl amino acids, etc.) and chemically derivatised amino acids (see below).

The term “blistering disease or blistering condition” as used herein refers to any disease or condition characterized by one or more blisters, in particular one or more blisters on the skin. A blister is a pocket of body fluid, commonly within the upper layers of skin. Blisters may be filled with serum, plasma, blood or, if infected, with pus. When, or if, a blister bursts or ruptures, wounds may be formed.

The term “dysregulated skin microbiome” as used herein refers to a skin microbiome characterized by excessive growth of microorganisms and/or a disruption of and/or an imbalance in the skin microbiome. For example, a dysregulated skin microbiome can have a reduced diversity of microbial species and/or an increased ratio of pathogenic or harmful, versus non-pathogenic or beneficial or benign, bacterial species as compared to a healthy skin microbiome. The term is used herein interchangeably with “dysfunctional skin microbiome”, “dysbiotic skin microbiome” and “dysregulated skin microbiota”.

The term “EDTA” as used herein refers to ethylenediaminetetraacetic acid.

The term “excessive wound exudate” as used herein refers to a high amount of wound exudate emitted from a wound. In particular, an excessive (i.e. high amount of) wound exudate may hinder or slow down wound healing. It may further result in that the individual suffering from excessive wound must change wound dressing very frequently, for example because wound fluid quickly saturates or even leaks from the wound dressing. Dressing leakage may increase the risk of infection.

The term “hydrogel” as used herein refers to a continuous phase of an aqueous solution and a hydrophilic polymer that is capable of swelling on contact with water. The “hydrogel” comprises nanostructures formed of said polymer and water, and typically contain more than 90% water. Hydrogels are typically transparent or translucent, regardless of their degree of hydration. Hydrogels are generally distinguishable from hydrocolloids, which typically comprise a hydrophobic matrix that contains dispersed hydrophilic particles. Hydrogels typically have a flow point of at least 10 Pa, such as at least 15 Pa, for example in the range of 10 to 80 Pa, such as in the range of 40 to 60 Pa.

The term “sequence identity” as used herein refers to the % of identical amino acids or nucleotides between a candidate sequence and a reference sequence following alignment. Thus, a candidate sequence sharing 80% amino acid identity with a reference sequence requires that, following alignment, 80% of the amino acids in the candidate sequence are identical to the corresponding amino acids in the reference sequence. Identity according to the present invention is determined by aid of computer analysis, such as, without limitations, the Clustal Omega computer alignment program for alignment of polypeptide sequences (Sievers et al. (2011 Oct. 11) Molecular Systems Biology 7:539, PMID: 21988835; Li et al. (2015 Apr. 6) Nucleic Acids Research 43 (W1): W580-4 PMID: 25845596; McWilliam et al., (2013 May 13) Nucleic Acids Research 41 (Web Server issue): W597-600 PMID: 23671338, and the default parameters suggested therein. The Clustal Omega software is available from EMBL-EBI at https://www.ebi.ac.uk/Tools/msa/clustalo/. Using this program with its default settings, the mature (bioactive) part of a query and a reference polypeptide are aligned. The number of fully conserved residues are counted and divided by the length of the reference polypeptide. The MUSCLE or MAFFT algorithms may be used for alignment of nucleotide sequences. Sequence identities may be calculated in a similar way as indicated for amino acid sequences. Sequence identity as provided herein is thus calculated over the entire length of the reference sequence.

The term “standard amino acid” refers to any of the twenty genetically-encoded amino acids commonly found in naturally occurring peptides. The standard amino acids are referred to herein both by their IUPAC 1-letter code and 3-letter code. The term “standard amino acid” is used to refer both to free standard amino acids, as well as standard amino acids incorporated into a peptide. For the peptides shown, each encoded amino acid residue, where appropriate, is represented by a single letter designation.

The term “topical administration” or “topically administering” as used herein refers to the application of a composition to the external surface of a patient, notably to the skin or mucosa. Desirably, the external surface is the skin and topical administration involves application of the composition to intact skin, to broken skin, to raw skin, to a blister or to an open skin wound.

The term “treatment” as used herein refers to any type of treatment or prevention of a disorder, including improvement in the disorder of the subject (e.g., in one or more symptoms), delay in the progression of the disorder, delay the onset of symptoms or slowing the progression of symptoms. Treatment may also be ameliorating or curative treatment. As such, the term “treatment” also includes prophylactic treatment of the individual to prevent the onset of symptoms, however treatment is preferably curative treatment, ameliorating treatment or treatment to alleviate the effect of one or more symptoms.

The term “wound exudate” as used herein refers to any fluid that filters from the circulatory system into wounds or blisters. Wound exudate may be serous, serosanguineous, sanguineous, hemorrhaging and/or purulent. It may comprise plasma, serum and/or various cell types such as neutrophils. The terms “exudate”, “wound exudate”, wound leakage” and “wound fluid” are used interchangeably herein. Wound exudate may be quantified by visual inspection of the wound dressing; by weighing the wound dressing; and/or by measuring the total protein content in the wound dressing, for example using the Bradford protein assay.

### Method of Treatment

The present invention relates a compound for use in a method of treating a skin disease or skin condition in an individual in need thereof, wherein said skin disease or skin condition is selected from the group consisting of a disease or condition characterized by blistering, a disease or condition characterized by excessive wound exudate, a disease or condition characterised by a dysregulated skin microbiome and scars,
wherein the compound comprises a peptide comprising or consisting of the amino acid sequence

X₁—X₂—X₃—X₄—X₅—X₆—W—X₈—X₉—X₁₀,

wherein
   X_{4, 6, 9} is any standard amino acid,
   X₁ is I, L or V,
   X₂ is any standard amino acid except C,
   X₃ is A, E, Q, R or Y,
   X₅ is any standard amino acid except R,
   X₈ is I or L,
   X₁₀ is any standard amino acid except H,
   wherein said peptide has a length of from 10 to 100 amino acid residues.

The present invention further relates to a method for treatment of a skin disease or skin condition in an individual in need thereof, wherein said skin disease or skin condition is selected from the group consisting of a disease or condition characterized by blistering, a disease or condition characterized by excessive wound exudate, a disease or condition characterised by a dysregulated skin microbiome and scars,
said method comprising administering a compound comprising a peptide comprising or consisting of the amino acid sequence

X₁—X₂—X₃—X₄—X₅—X₆—W—X₈—X₉—X₁₀,

wherein
   X_{4, 6, 9} is any standard amino acid,
   X₁ is I, L or V,
   X₂ is any standard amino acid except C,
   X₃ is A, E, Q, R or Y,
   X₅ is any standard amino acid except R,
   X₈ is I or L,
   X₁₀ is any standard amino acid except H,
   wherein said peptide has a length of from 10 to 100 amino acid residues.

The present invention further relates to a pharmaceutical composition for use in the treatment of a skin disease or skin condition in an individual in need thereof, wherein said skin disease or skin condition is selected from the group consisting of a disease or condition characterized by blistering, a disease or condition characterized by excessive wound exudate, a disease or condition characterised by a dysregulated skin microbiome and scars,
wherein the composition comprises a compound comprising a peptide comprising or consisting of the amino acid sequence

X₁—X₂—X₃—X₄—X₅—X₆—W—X₈—X₉—X₁₀,

wherein
   X_{4, 6, 9} is any standard amino acid,
   X₁ is I, L or V,
   X₂ is any standard amino acid except C,
   X₃ is A, E, Q, R or Y,
   X₅ is any standard amino acid except R,
   X₈ is I or L,
   X₁₀ is any standard amino acid except H,
   wherein said peptide has a length of from 10 to 100 amino acid residues.

The present invention further relates to use of a compound in the manufacture of a medicament for the treatment of a skin disease or skin condition in an individual in need thereof, wherein said skin disease or skin condition is selected from the group consisting of a disease or condition characterized by blistering, a disease or condition characterized by excessive wound exudate, a disease or condition characterised by a dysregulated skin microbiome and scars,
wherein the compound comprises a peptide comprising or consisting of the amino acid sequence

X₁—X₂—X₃—X₄—X₅—X₆—W—X₈—X₉—X₁₀,

wherein
   X_{4, 6, 9} is any standard amino acid,
   X₁ is I, L or V,
   X₂ is any standard amino acid except C,
   X₃ is A, E, Q, R or Y,
   X₅ is any standard amino acid except R,
   X₈ is I or L,
   X₁₀ is any standard amino acid except H,
   wherein said peptide has a length of from 10 to 100 amino acid residues.

The present invention further relates to a compound for use in a method of reducing the amount of fluid that exudes from one or more wound or blister in an individual in need thereof,
wherein the compound comprises a peptide comprising or consisting of the amino acid sequence

X₁—X₂—X₃—X₄—X₅—X₆—W—X₈—X₉—X₁₀,

wherein
   X_{4, 6, 9} is any standard amino acid,
   X₁ is I, L or V,
   X₂ is any standard amino acid except C,
   X₃ is A, E, Q, R or Y,
   X₅ is any standard amino acid except R,
   X₈ is I or L,
   X₁₀ is any standard amino acid except H,
   wherein said peptide has a length of from 10 to 100 amino acid residues.

The present invention further relates to a method of reducing the amount of fluid that exudes from one or more wound or blister in an individual in need thereof,
said method comprising administering a compound comprising a peptide comprising or consisting of the amino acid sequence

X₁—X₂—X₃—X₄—X₅—X₆—W—X₈—X₉—X₁₀,

wherein
   X_{4, 6, 9} is any standard amino acid,
   X₁ is I, L or V,
   X₂ is any standard amino acid except C,
   X₃ is A, E, Q, R or Y,
   X₅ is any standard amino acid except R,
   X₈ is I or L,
   X₁₀ is any standard amino acid except H,
   wherein said peptide has a length of from 10 to 100 amino acid residues.

The present invention further relates to a compound for use in a method of treatment of a skin disease or skin condition in an individual in need thereof, wherein the skin disease or skin condition is a wound or a disease or condition characterized by blistering, and wherein the treatment reduces the amount of fluid that exudes from one or more wound or blister, and wherein the compound comprises a peptide comprising or consisting of the amino acid sequence

X₁—X₂—X₃—X₄—X₅—X₆—W—X₈—X₉—X₁₀,

wherein
X_{4, 6, 9} is any standard amino acid,
X₁ is I, L or V,
X₂ is any standard amino acid except C,
X₃ is A, E, Q, R or Y,
X₅ is any standard amino acid except R,
X₈ is I or L,
X₁₀ is any standard amino acid except H,
wherein said peptide has a length of from 10 to 100 amino acid residues.

The present invention further relates to a method of treatment of a skin disease or skin condition in an individual in need thereof, wherein the skin disease or skin condition is a wound or a disease or condition characterized by blistering, and wherein the treatment reduces the amount of fluid that exudes from one or more wound or blister, said method comprising administering a compound comprising a peptide comprising or consisting of the amino acid sequence

X₁—X₂—X₃—X₄—X₅—X₆—W—X₈—X₉—X₁₀,

wherein
X_{4, 6, 9} is any standard amino acid,
X₁ is I, L or V,
X₂ is any standard amino acid except C,
X₃ is A, E, Q, R or Y,
X₅ is any standard amino acid except R,
X₈ is I or L,
X₁₀ is any standard amino acid except H,
wherein said peptide has a length of from 10 to 100 amino acid residues.

The present invention further relates to a compound for promoting a healthy skin microbiome in an individual in need thereof,
wherein the compound comprises a peptide comprising or consisting of the amino acid sequence

X₁—X₂—X₃—X₄—X₅—X₆—W—X₈—X₉—X₁₀,

wherein
   X_{4, 6, 9} is any standard amino acid,
   X₁ is I, L or V,
   X₂ is any standard amino acid except C,
   X₃ is A, E, Q, R or Y,
   X₅ is any standard amino acid except R,
   X₈ is I or L,
   X₁₀ is any standard amino acid except H,
   wherein said peptide has a length of from 10 to 100 amino acid residues.

The present invention further relates to a method for promoting a healthy skin microbiome in an individual in need thereof,
said method comprising administering a compound comprising a peptide comprising or consisting of the amino acid sequence

X₁—X₂—X₃—X₄—X₅—X₆—W—X₈—X₉—X₁₀,

wherein
   X_{4, 6, 9} is any standard amino acid,
   X₁ is I, L or V,
   X₂ is any standard amino acid except C,
   X₃ is A, E, Q, R or Y,
   X₅ is any standard amino acid except R,
   X₈ is I or L,
   X₁₀ is any standard amino acid except H,
   wherein said peptide has a length of from 10 to 100 amino acid residues.

In some embodiments, the individual suffers from a skin disease or skin condition selected from the group consisting of a disease or condition characterized by blistering, a disease or condition characterized by excessive wound exudate, a disease or condition characterised by a dysregulated skin microbiome and scars.

In some embodiments, the individual suffers from a skin disease or skin condition selected from the group consisting of burn wounds, chronic skin ulcers, infected acute wounds, pressure ulcers and chronic wounds such as venous ulcers, for example venous leg ulcers, or diabetic leg ulcers. In such embodiments, the skin disease or skin condition is preferably any of the aforementioned, wherein said skin disease or condition is associated with excessive wound exudate or associated with a dysregulated skin microbiome.

### Disease or Condition Characterized by Blisters

A blister is a pocket of body fluid, commonly within the upper layers of skin. Blisters may be filled with serum, plasma, blood or, if infected, with pus. When, or if, a blister bursts or ruptures, wounds may be formed. The disease or condition characterized by blisters as defined herein may be any type of blistering disease or condition.

In some embodiments, the disease or condition characterized by blistering is selected from the group consisting of:
epidermolysis bullosa (EB),
burn-induced blisters,
chilblains,
cryotherapy-induced blisters,
virus-induced blisters, such as chickenpox, shingles, herpes simplex, atypical enterovirus infection, hand, foot and mouth disease, eczema herpeticum, bullous impetigo and staphylococcal scalded skin syndrome,
pemphigus, such as pemphigus foliaceous, pemphigus vulgaris, paraneoplastic pemphigus, pemphigus erythematosus or pemphigus vegetans,
pemphigoid, such as bullous pemphigoid, cicatricial pemphigoid, 200 k pemphigoid or mucous membrane pemphigoid,
linear IgA bullous dermatosis,
dermatitis herpetiformis,
Porphyria cutanea tarda,
bullous lupus erythematosus,
Hailey-Hailey disease and
Steven Johnson syndrome (toxic epidermal necrolysis).

In some embodiments, the disease or condition characterized by blistering is burn-induced blisters, such as blisters from second-degree superficial partial thickness burns or second-degree deep partial thickness burns.

In one embodiment, the blistering disease or condition is characterized by excessive wound exudate, such that leakage of wound fluid is a symptom of the blistering disease or condition.

In one embodiment, the blistering disease or condition is characterized by a dysregulated skin microbiome, such that dysregulated skin microbiome is a symptom of the blistering disease or condition.

In one embodiment, the blistering disease or condition is characterized by scars, such that scar formation is a symptom of the blistering disease or condition.

### Epidermolysis Bullosa

In preferred embodiments, the disease or condition characterized by blistering is epidermolysis bullosa (EB). Thus, in a preferred embodiment the present invention relates to the compound comprising a peptide described herein for use in a method for treatment of EB. Hence, in one embodiment the invention relates to a compound for use in a method of treating EB in an individual in need thereof, wherein the compound comprises a peptide comprising or consisting of the amino acid sequence:

X₁—X₂—X₃—X₄—X₅—X₆—W—X₈—X₉—X₁₀,

wherein
X_{4, 6, 9} is any standard amino acid,
X₁ is I, L or V,
X₂ is any standard amino acid except C,
X₃ is A, E, Q, R or Y,
X₅ is any standard amino acid except R,
X₈ is I or L,
X₁₀ is any standard amino acid except H,
   wherein said peptide has a length of from 10 to 100 amino acid residues.

EB is a rare genetic disease characterized by blistering and wound formation in the skin and mucous membranes following minimal trauma. There are an estimated 500 000 cases worldwide, which are caused by different mutations. At least 20 different genes coding for components contributing to the adhesion and anchorage of the skin and mucous membrane have been described to be involved in different kinds of EB. There is no cure for the condition, and treatment may instead focus on wound care and treating symptoms associated with the wounds such as pain, itch, or infection.

There are several different types of EB. The peptides of the invention may be used for treatment of any type of EB. Thus, in some embodiments of the invention, the EB is inherited epidermolysis bullosa. In other embodiments, the EB is acquired epidermolysis bullosa. In some embodiments, the EB is epidermolysis bullosa simplex, junctional epidermolysis bullosa, dystrophic epidermolysis bullosa, such as dominant dystrophic epidermolysis bullosa or recessive dystrophic epidermolysis bullosa, Kindler syndrome or epidermolysis bullosa acquisita.

Blisters may occur in all types of EB following friction and/or relatively minor trauma. They may be present anywhere on the skin and the mucous membranes. Commonly, blister formation in EB is the first sequence in wound generation, the blisters containing exudate. The wounds formed after the blisters open may produce an excessive amount of exudate. Dysregulated skin microbiome and scarring, as well as infection and inflammation of the wounds, may also be prevalent features of EB. The methods of the invention may be used to treat any such blisters, and/or to reduce excessive amount of exudate and/or to restore a healthy microbiome in EB patients.

In some embodiments, the method of treatment disclosed herein in the section “Method of treatment” reduces the size of one or more blisters or wounds in an individual suffering from EB.

In some embodiments, treatment reduces the size of one or more blister or wound by at least 10%, such as at least 20%, such as at least 30%, such as at least 35%, such as at least 40%, such as at least 45%, such as at least 50%, such as at least 60%, such as at least 70%, such as at least 80%, such as at least 90%, such as wherein the treatment reduces the size of one or more blister or wound by at least 95% as compared to the size of the one or more blister or wound before treatment, or as compared to the size of a blister or wound which was not treated with the compound comprising the peptide, and which before treatment was of similar size and character as the one or more treated blister or wound.

In some embodiments, treatment reduces the size of one or more blister or wound by between 10% and 100%, such as by between 20% and 100%, such as by between 30% and 100%, such as by between 40% and 100%, such as by between 50% and 100%, such as by between 60% and 100%, such as by between 70% and 100%, such as by between 80% and 100%, such as by between 90% and 100%, such as by between 10% and 90%, such as by between 20% and 90%, such as by between 30% and 90%, such as by between 40% and 90%, such as by between 50% and 90%, such as by between 60% and 90%, such as by between 70% and 90%, such as by between 80% and 90%, such as by between 10% and 80%, such as by between 20% and 80%, such as by between 30% and 80%, such as by between 40% and 80%, such as by between 50% and 80%, such as by between 60% and 80%, such as by between 70% and 80%, such as by between 10% and 70%, such as by between 20% and 70%, such as by between 30% and 70%, such as by between 40% and 70%, such as by between 50% and 70%, such as by between 60% and 70%, such as by between 10% and 60%, such as by between 20% and 60%, such as by between 30% and 60%, such as by between 40% and 60%, such as by between 50% and 60%, such as by between 10% and 50%, such as by between 20% and 50%, such as by between 30% and 50%, such as by between 40% and 50%, such as by between 10% and 40%, such as by between 20% and 40%, such as by between 30% and 40%, such as by between 10% and 30%, such as by between 20% and 30%, such as by between 10% and 20%.

In some embodiments, treatment reduces the size of one or more blister or wound by at least 10%, such as at least 20%, such as at least 30%, such as at least 40%, such as at least 50%, such as at least 60%, such as at least 70%, such as at least 80%, such as at least 90%.

In some embodiments, treatment reduces the size of one or more blister or wound by 100%, such that the treatment results in that one or more blister or wound heals completely.

In some embodiments, the size of one or more blister or wound is reduced after treatment for at least two days, such as at least five days, such as at least one week, such as at least two weeks, such as at least three weeks, such as at least four weeks, such as at least one month, such as at least two months, such as at least five months, such as at least six months, such as at least seven months, such as at least eight months, such as at least nine months, such as at least ten months, such as at least eleven months, such as wherein the size of one or more blister or wound is reduced after one year of treatment.

In some embodiments, the size of one or more blister or wound is reduced after treatment for between two days and two years, such as between five days and one year, such as between one month and one year, such as between two days and one month, such as between two weeks and six months, such as between one week and three months, such as between two months and one year, such as between one month and four months, such as between two weeks and eight weeks, such as between one week and five weeks.

In some embodiments, the wound or blister stays healed and does not reopen for at least two days, such as at least five days, such as at least one week, such as at least two weeks, such as at least three weeks, such as at least four weeks, such as at least one month, such as at least two months, such as at least five months, such as at least six months, such as at least seven months, such as at least eight months, such as at least nine months, such as at least ten months, such as at least eleven months, such as at least one year, such as at least two years, such as at least three years, such as at least four years, such as at least five years after the treatment is completed, such as wherein the wound or blister stays healed and does reopen at all after the treatment is completed.

In some embodiments, the wound or blister stays healed and does not reopen for between two days and five years, such as between one week and one months, such as between two months and eight months, such as between one year and five years.

EB may be associated with a pain, such that pain is a symptom of EB. In some embodiments, treatment reduces the pain experienced by the individual, such as wherein said treatment reduces the pain of one or more wound or blister.

Pain may be measured using a pain scale. In some embodiments, pain is measured using a self-reporting pain scale. In other embodiments, pain is measured using an observer pain scale. In other words, in some embodiments, treatment reduces the pain experienced by the individual, wherein said reduction in pain is measured using a pain scales.

In some embodiments, pain is measured using a pain scale selected from the group consisting of Neonatal/Infants Pain Scale (NIPS), Children's Hospital of Eastern Ontario Pain Scale (CHEOPS), Face, Legs, Activity, Crying, Consolability (FLACC) scale, Faces Pain Scale (Wong-Baker Faces Pain Rating Scale), Coloured Analogue Scale, Visual Analog Scale, Verbal Numerical Rating Scale and Wharton Impairment and Pain Scale.

In some embodiments, pain is reduced at least one step or score on a pain scale, such as at least two steps or scores, such as at least three steps or scores, such as at least four steps or scores, such as at least five steps or scores, such as at least six steps or scores, such as at least seven steps or scores, such as at least eight steps or scores, such as at least nine steps or scores, such as wherein the pain is reduced ten steps or scores on a pain scale, such as on any one of the pain scales mentioned herein above.

In some embodiments, pain is reduced between one and ten steps or scores on a pain scale, such as between one and nine steps or scores, such as between one and eight steps or scores, such as between one and seven steps or scores, such as between one and six steps or scores, such as between one and five steps or scores, such as between one and four steps or scores, such as between one and three steps or scores, such as between one and two steps or scores, such as between two and ten steps or scores, such as between two and nine steps or scores, such as between two and eight steps or scores, such as between two and seven steps or scores, such as between two and six steps or scores, such as between two and five steps or scores, such as between two and four steps or scores, such as between two and three steps or scores, such as between three and ten steps or scores, such as between three and nine steps or scores, such as between three and eight steps or scores, such as between three and seven steps or scores, such as between three and six steps or scores, such as between three and five steps or scores, such as between three and four steps or scores, such as between four and ten steps or scores, such as between four and nine steps or scores, such as between four and eight steps or scores, such as between four and seven steps or scores, such as between four and six steps or scores, such as between four and five steps or scores, such as between five and ten steps or scores, such as between five and nine steps or scores, such as between four and eight steps or scores, such as between four and seven steps or scores, such as between four and six steps or scores, such as between five and six steps or scores, such as between six and ten steps or scores, such as between six and nine steps or scores, such as between six and eight steps or scores, such as between six and seven steps or scores, such as between seven and ten steps or scores, such as between seven and nine steps or scores, such as between seven and eight steps or scores, such as between eight and ten steps or scores, such as between eight and nine steps or scores, such as wherein pain is reduced between nine and ten steps or scores on a pain scale.

EB may also be associated with itch, such that itch is a symptom of EB. In some embodiments, treatment reduces the itch experienced by the individual, such as wherein said treatment reduces the itch of one or more wound or blister.

Itch may be determined using subjective patient reporting or by objective measurement of scratching activity or scratching induced skin changes. In some embodiments, itch is measured using the Leuven Itch Scale. In some embodiments, itch is measured using the 5-D itch scale.

EB may also be associated with excessive wound leakage, such that leakage from the EB wounds is a symptom of EB. In particular embodiments, the individual suffering from EB has one or more wounds that leak fluid, i.e. one or more wounds with excessive wound exudate, such as described in the section “Disease or condition characterized by wound exudate”.

Thus, in one embodiment treatment reduces wound leakage in a patient suffering from EB, for example as described in the section “Disease or condition characterized by wound exudate”.

EB may also be associated with a dysregulated skin microbiome, such that a dysregulated skin microbiome is a symptom of EB. In particular embodiments, the individual suffering from EB has a dysregulated skin microbiome in one or more wounds, such as described in the section “Disease or condition characterized by dysregulated skin microbiome”. Such dysregulated skin microbiome may hamper wound healing and increase the risk of infection or even sepsis.

Thus, in one embodiment, treatment promotes a healthy skin microbiome in a patient suffering from EB, such as described in the section “Disease or condition characterized by dysregulated skin microbiome”. In one embodiment, treatment reduces the total amount of microorganisms, such as the total amount of commensal microorganisms, present on the skin and/or in on or more wounds of a patient suffering from EB, such as described in the section “Disease or condition characterized by dysregulated skin microbiome”. In one embodiment, increases the ratio of beneficial:harmful microbial species and/or strains in the skin microbiome, of the skin and/or in on or more wounds of a patient suffering from EB, such as described in the section “Disease or condition characterized by dysregulated skin microbiome”.

EB may also be associated with scars, such that scar formation is a symptom of EB. In particular embodiments, the individual suffering from EB has one or more scars that have been formed from the one or more wounds, such as described in the section “Scars”.

Thus, in one embodiment treatment reduces scar formation in a patient suffering from EB, for example as described in the section “Scars”.

### Disease or Condition Characterized by Wound Exudate

Wound exudate or wound fluid may be any fluid that filters from the circulatory system into wounds or blisters. Wound exudate may be serous, serosanguineous, sanguineous, hemorrhaging and/or purulent. It may comprise plasma, serum and/or various cell types such as neutrophils. In certain wounds, the amount of wound exudate may be at such a high level that it impairs healing of the wound. In other words, excessive wound exudate may slow down or worsen wound healing. Thus, decreasing the amount of wound exudate may improve healing of the wound.

The amount of wound exudate emitted from a wound may be independent of the amount of bacteria colonizing the wound. This is for example evident by the fact that the peptides of the present invention decreases bacterial levels in a wound in a dose-independent manner, while decrease of wound leakage appears to be dose-dependent.

The amount of fluid leaking from a wound may be reduced in any individual in need thereof, such as in any individual suffering from a disease or condition characterized by excessive wound exudate. The individual may for example suffer a disease or condition characterized by blistering, as described herein in the section “Disease or condition characterized by blistering”. In one embodiment, the individual suffers from epidermolysis bullosa as described herein in the section “Epidermolysis bullosa”.

In some embodiments, however, the individual suffers from a skin disease or skin condition selected from the group consisting of burn wounds, chronic skin ulcers, infected acute wounds, pressure ulcers and chronic wounds such as venous ulcers, for example venous leg ulcers, or diabetic leg ulcers.

In some embodiments, treatment reduces the amount of fluid that exudes from one or more blister or wound, such as wherein treatment reduces the amount of wound exudate.

In some embodiments, treatment decreases the amount of fluid that exudes from a wound or blister by at least 10%, such as at least 20%, such as at least 30%, such as at least 35%, such as at least 40%, such as at least 45%, such as at least 50%, such as at least 60%, such as at least 70%, such as at least 80%, such as at least 90%, such as wherein the amount of wound exudate is decreased by at least 95%.

In some embodiments, treatment decreases the amount of fluid that exudes from a wound or blister by between 10% and 95%, such as between 10% and 90%, such as between 15% and 80%, such as between 10% and 40%, such as between 30% and 80%, such as between 50% and 90%, such as between 20% and 50%, such as between 40% and 60%, such as between 10% and 25%, such as between 30% and 70%, such as between 80% and 90%, such as between 70% and 80% such as between 50% and 70% such as between 20% and 70%.

In some embodiments, treatment decreases the amount of fluid that exudes from a wound or blister at least one step or score on an exudate scale, such as at least two steps or scores, such as at least three steps or scores, such as at least four steps or scores, such as at least five steps or scores on a wound exudate scale.

In one embodiment, scoring or scaling of exudate, i.e. leakage, is carried out by visually inspecting the wound dressing after a certain amount of time, for example using a visual scoring scale from 0-5, where 0 represents “No exudation in wound area” and 5 represents “Exudation soaking the dressing in an area exceeding the wound area”.

In some embodiments, the decrease is as compared to before starting treatment of the wound or blister. In some embodiments, the decrease is as compared to a wound or blister of similar size and character which was not treated with the compound comprising the peptide and which before treatment had a similar amount of wound exudate as the treated wound or blister.

In order to quantify wound exudate, the wound exudate may be collected in wound dressing(s), and the amount collected in said wound dressing(s) may be quantified.

In some embodiments, the amount of wound exudate is estimated by visual inspection of the wound dressing.

In some embodiments, the amount of wound exudate is measured by imaging of the wound dressing.

In some embodiments, the amount of wound exudate is measured by weighing the wound dressing.

In some embodiments, the amount of wound exudate is measured by measuring the protein content in the wound dressing, such as the total protein content in the wound dressing. Thus, the total protein content in a wound dressing is correlated to wound exudate and can therefore be used as a quantification of wound exudate. In some embodiments, the protein content is measured using the Bradford protein assay and/or using any other method well known in the art.

Thus, in some embodiments, treatment decreases the total protein content that exudes from a wound or blister.

In preferred embodiments, wound exudate is measured as describe in Example 1 herein below.

In some embodiments, treatment reduces the amount of heparin-binding protein (HBP) in the wound exudate. HBP, also known as azurocidin or cationic antimicrobial protein of 37 kDa, is an inflammatory mediator with the ability to induce vascular leakage. In one embodiment, the amount of HBP is decreased at least 20%, such as at least 30%, such as at least 40%, such as at least 50%, such as at least 60%, such as at least 70%, such as at least 80%, such as at least 90%. In one embodiment, the amount of HBP is decreased between 20% and 90%, such as between 30% and 80%, such as between 50% and 90%, such as between 60% and 75%.

The amount of HPB in wound exudate can be determined using methods known in the art, for example using the methods described in Example 1 or Example 5 of the present application.

Excessive leakage of wound fluid from one or more wounds may result in that the individual suffering from said one or more wounds must change wound dressing at regular intervals. Changing wound dressing may, in particular for individuals suffering from EB or other painful wounds, be associated with a high degree of pain and suffering. Reducing the amount of fluid leaking from one or more wounds may result in that the dressing does not need to be changed as often.

Thus, in one embodiment, treatment reduces the amount of times that wound dressings need to be changed within a set time period, such as wherein treatment increases the amount of days passed before the wound dressings must be changed, such as wherein treatment results in wound dressings not needing to be changed as often as compared to before starting treatment.

In one embodiment, treatment increases the amount of days passed between changing wound dressings with half a day, such as with one day, such as with 1.5 days, such as with 2 days, such as with 2.5 days, such as with 3 days, such as with 3.5 days, such as with 4 days, such as with 4.5 days, such as with 5 days, such as wherein treatment increases the amount of days passed between changing wound dressings with 6 days.

In one embodiment, after and/or during treatment according to the invention the amount of days passed between need for changing wound dressings is between one day and six days, such as between one day and five days, such as between one day and four days, such as between one day and three days, such as between one day and two days, such as between two days and five days, such as between two days and four days.

In one embodiment, treatment results in that the wound dressing only needs to be changed every third day, such as every fourth day, such as every fifth day, such as every sixth day, such as wherein treatment results in that wound dressing only needs to be changed once per week.

The timepoint for changing the wound dressing may be determined by any person familiar with determining said timepoint, such as a caregiver, a physician, a nurse and/or the individual suffering from the disease or condition. Thus, in some embodiments, the timepoint for changing the wound dressing is determined by visual inspection of the wound dressing, such as by visual inspection by a person familiar with determining when the wound dressing needs to be changed.

In some embodiments, the timepoint for changing the wound dressing is determined based on the amount of exudate present in the dressing.

In some embodiments the timepoint for changing the wound dressing is determined to reduce the risk of inflammation and/or infection, for example wherein the timepoint is predetermined in order to reduce the risk of inflammation and/or infection.

### Disease or Condition Characterized by Dysregulated Skin Microbiome

The skin microbiome, also called the skin flora or the skin microbiota, refers to the community of microorganisms that reside on the skin. Commonly, the majority of the skin microbiome is comprised of bacteria, such as up to around 1,000 species of bacteria from 19 phyla. A healthy skin microbiome is non-pathogenic, and may comprise commensal and/or mutualistic bacteria. A healthy skin microbiome may prevent pathogenic bacteria from colonizing the skin, for example by competing with said pathogenic bacteria for nutrients, and/or by stimulating the skin's immune system. In some instances, a healthy skin microbiome may promote and/or improve wound healing.

Many skin diseases and conditions are associated with alterations and changes of the healthy skin microbiome, i.e. a dysbiosis or dysregulation of the skin microbiome. The dysregulated skin microbiome may be characterized by overgrowth or excessive growth of skin microorganisms and/or an increased ratio of pathogenic, i.e. harmful, bacterial species as compared to the ratio of said species in a healthy skin microbiome. In some instances, dysbiosis is characterized by excessive growth of commensal and non-pathogenic bacteria. In some instances, bacteria colonizing the skin microbiome may turn pathogenic and cause disease. In some instances, damaged skin may cause non-pathogenic bacteria to become pathogenic. Reversion of skin microbiome dysregulation may help to prevent and/or treat the skin disease or condition.

The present invention provides peptides for promoting a healthy skin microbiome and/or for treating a dysregulated skin microbiome. Thus, in one embodiment, treatment promotes a healthy skin microbiome.

A dysregulated skin microbiome may be treated and/or a healthy skin microbiome may be promoted in any individual in need thereof, such as in any individual suffering from a disease or condition characterized by dysregulated skin microbiome. The individual may for example suffer a disease or condition characterized by blistering, as described herein in the section “Disease or condition characterized by blistering”.

In some embodiments, however, the individual suffers from a skin disease or skin condition selected from the group consisting of burn wounds, chronic skin ulcers, infected acute wounds, pressure ulcers and chronic wounds such as venous ulcers, for example venous leg ulcers, or diabetic leg ulcers, wherein said wounds are characterized by a dysregulated skin microbiome.

In other embodiments, the individual suffers from eczema, such as of atopic dermatitis. Thus, in some embodiments, a healthy skin microbiome is promoted in an individual suffering from atopic dermatitis.

As stated herein above, the dysregulated microbiome may be characterised by an increased ratio of harmful microorganisms in comparison to beneficial microorganisms, i.e. an increased ratio of (potentially) pathogenic microorganisms in comparison to non-pathogenic microorganisms. In other words, the dysregulated microbiome may be characterised by a decreased ratio of beneficial microorganisms in comparison to harmful microorganisms, i.e. an increased ratio of (potentially) pathogenic microorganisms in comparison to non-pathogenic microorganisms. Said microorganisms may be of a certain microbial strain and/or species.

Thus, in one embodiment, the dysregulated skin microbiome is characterised by a decreased ratio of beneficial:harmful microbial species and/or strains as compared to the ratio of beneficial:harmful microbial species and/or strains in an individual not suffering from a dysregulated microbiome, such that the harmful microbial species and/or strains comprise a larger part of the total skin microbiome in the dysregulated skin microbiome as compared to the skin microbiome of an individual not suffering from a dysregulated skin microbiome.

The dysregulated skin microbiome may be also be characterized by overgrowth or excessive growth of skin microorganisms. In such embodiments, it may be important to decrease the bacterial load of the wound, for example to prevent colonization of the wound or blister by pathogenic bacteria, such as to prevent e.g. infection of the wound. Thus, in some embodiments, treatment reduces the total amount of microorganisms, such as wherein treatment reduces an excessive amount of microorganisms, for example in one or more wound or blister, such as the total amount of bacteria present in one or more wound or blister.

In some embodiments, treatment reduces the total amount of skin microorganisms in the skin microbiome, such as wherein treatment reduces the overall bacterial load in a wound or blister.

In some embodiments, one or more of the skin microorganisms are resistant to one or more antibiotics. Thus, in some embodiments, treatment reduces the total amount of skin microorganisms of a skin microbiome comprising one or more skin microorganisms that are resistant to one or more antibiotics.

In some embodiments, treatment reduces the total amount of microorganisms present in one or more wound or blister by at least 10%, such as at least 20%, such as at least 30%, such as at least 35%, such as at least 40%, such as at least 45%, such as at least 50%, such as at least 60%, such as at least 70%, such as at least 80%, such as at least 90%, such as wherein the total amount of microorganisms present in one or more wound or blister is reduced by at least 95% as compared to the amount of bacteria present before treatment.

In some embodiments, treatment reduces the total amount of microorganisms present in one or more wound or blister by between 10% and 95%, such as between 10% and 90%, such as between 15% and 80%, such as between 10% and 40%, such as between 30% and 80%, such as between 50% and 90%, such as between 20% and 50%, such as between 40% and 60%, such as between 10% and 25%, such as between 30% and 70%, such as between 80% and 90%, such as between 70% and 80% such as between 50% and 70% such as between 20% and 70%.

The amount of bacteria may be measured using any technique well known in the art. For example, the amount of microorganisms is measured by measuring the amount of colony-forming units (CFU) present in a wound dressing and/or in a wound or blister on the skin. In some embodiments, said measurement takes place before, during and/or after treatment.

However, it may also be important to maintain the beneficial, i.e. the non-pathogenic, bacteria in the microbiome of the wound. In other words, it may be important to maintain the bacterial diversity of the wound to prevent subsequent colonization by pathogenic bacteria after treatment is completed.

Thus, in some embodiments, treatment preserves the composition of the skin microbiome, such as wherein treatment preserves the microbial diversity of the skin microbiome.

In some embodiments, treatment preserves the ratio between different microbial species and/or strains in the skin microbiome, such as that the ratio between different microbial species and/or strains in the skin microbiome is the same or similar before, during and/or after treatment. In other words, in some embodiments, treatment maintains the ratio of beneficial:harmful microorganisms in the skin microbiome, such as wherein the ratio of beneficial:harmful microorganisms in the skin microbiome is the same or similar before, during and/or after treatment.

In some embodiments, however, treatment increases the ratio of beneficial:harmful microorganisms in the skin microbiome, for example wherein treatment doubles, triples or quadruples the ratio of beneficial:harmful microorganisms in the skin microbiome, such that the beneficial microorganisms during and/or after treatment make up a larger part of the total skin microbiome relative to the harmful microbial microorganisms.

The ratio may be calculated by dividing the amount of beneficial microorganisms with the amount of harmful microorganisms. In some embodiments, the ratio is calculated by dividing the amount of at least one beneficial microorganism with the amount of at least one harmful microorganism, such as the amount of at least two beneficial microorganisms with the amount of at least two harmful microorganisms, such as the amount of at least three beneficial microorganisms with the amount of at least three harmful microorganisms, such as the amount of at least four beneficial microorganisms with the amount of at least four harmful microorganisms, such as the amount of at least five beneficial microorganisms with the amount of at least five harmful microorganisms, such as the amount of at least ten beneficial microorganisms with the amount of at least ten harmful microorganisms.

In some embodiments, the ratio is calculated by dividing the amount of between 1 and 100 beneficial microorganism with the amount of between 1 and 100 harmful microorganism, such as the amount between 1 and 50 beneficial microorganism with the amount of between 1 and 50 harmful microorganism, such as the amount between 1 and 20 beneficial microorganism with the amount of between 1 and 20 harmful microorganism, such as the amount between 1 and 10 beneficial microorganism with the amount of between 1 and 10 harmful microorganism, such as the amount between 2 and 10 beneficial microorganism with the amount of between 2 and 10 harmful microorganism, such as the amount between 2 and 5 beneficial microorganism with the amount of between 2 and 5 harmful microorganism, such as the amount between 10 and 15 beneficial microorganism with the amount of between 10 and 15 harmful microorganism, such as the amount between 2 and 50 beneficial microorganism with the amount of between 2 and 50 harmful microorganism, such as the amount between 3 and 10 beneficial microorganism with the amount of between 3 and 10 harmful microorganism.

In some embodiments, the ratio is calculated based on at least one beneficial microorganism, such as at least two beneficial microorganisms, such as at least three beneficial microorganisms, such as at least four beneficial microorganisms, such as at least five beneficial microorganisms, such as at least six beneficial microorganisms, such as at least seven beneficial microorganisms, such as at least eight beneficial microorganisms, such as at least nine beneficial microorganisms, such as at least ten beneficial microorganisms, such as at least 15 beneficial microorganisms, such as at least 20 beneficial microorganisms, such as at least 25 beneficial microorganisms, such as at least 30 beneficial microorganisms, such as at least 40 beneficial microorganisms, such as at least 50 beneficial microorganisms, such as at least 100 beneficial microorganisms.

In some embodiments, the ratio is calculated based on between 1 and 500 beneficial microorganisms, such as between 1 and 300 beneficial microorganisms, such as between 1 and 50 beneficial microorganisms, such as between 1 and 10 beneficial microorganisms, such as between 5 and 100 beneficial microorganisms, such as between 5 and 50 beneficial microorganisms, such as between 2 and 20 beneficial microorganisms, such as between 1 and 5 beneficial microorganisms, such as between 2 and 4 harmful microorganisms.

In some embodiments, the ratio is calculated based on at least one harmful microorganism, such as at least two harmful microorganisms, such as at least three harmful microorganisms, such as at least four harmful microorganisms, such as at least five harmful microorganisms, such as at least six harmful microorganisms, such as at least seven harmful microorganisms, such as at least eight harmful microorganisms, such as at least nine harmful microorganisms, such as at least ten harmful microorganisms, such as at least 15 harmful microorganisms, such as at least 20 harmful microorganisms, such as at least 25 harmful microorganisms, such as at least 30 harmful microorganisms, such as at least 40 harmful microorganisms, such as at least 50 harmful microorganisms, such as at least 100 harmful microorganisms.

In some embodiments, the ratio is calculated based on between 1 and 500 harmful microorganisms, such as between 1 and 300 harmful microorganisms, such as between 1 and 50 harmful microorganisms, such as between 1 and 10 harmful microorganisms, such as between 5 and 100 harmful microorganisms, such as between 5 and 50 harmful microorganisms, such as between 2 and 20 harmful microorganisms, such as between 1 and 5 harmful microorganisms, such as between 2 and 4 harmful microorganisms.

The harmful, i.e. pathogenic microorganisms may be any microorganism known in the art to be pathogenic and/or harmful at high levels. The beneficial, i.e. non-pathogenic microorganisms may be any microorganism known in the art to be non-pathogenic. Thus, that ratio may be calculated on any microorganisms known in the art to be pathogenic or non-pathogenic.

In some embodiments, the harmful microorganism is a gram-negative bacteria. In some embodiments, the harmful microorganism is selected from the group consisting of Staphylococcus aureus, Bacillus licheniformis, Bacillus cereus complex, Rothia amarae, Staphylococcus spp., Streptococcus spp., group A streptococci, such as Streptococcus pyogenes, and gram-negative bacteria, such as Pseudomonas aeruginosa, Proteobacteria spp. And Proteus spp.

In some embodiments, the harmful microorganism is at least one microorganism, such as at least two microorganisms, such as at least three microorganisms, such as at least four microorganisms, such as at least five microorganisms, such as at least six microorganisms, such as at least seven microorganisms, such as at least eight microorganisms, such as at least nine microorganisms, such as at least ten microorganisms, such as at least eleven microorganisms, such as at least twelve microorganisms, such as at least 13 microorganisms, such as at least 14 microorganisms, such as at least 15 microorganisms, such as at least 25 microorganisms, such as all microorganisms selected from the group consisting of Staphylococcus aureus, Bacillus licheniformis, Bacillus cereus complex, Rothia amarae, Staphylococcus spp., Streptococcus spp., group A streptococci, such as Streptococcus pyogenes, and gram-negative bacteria, such as Pseudomonas aeruginosa, Proteobacteria spp. And Proteus spp.

In some embodiments, the beneficial microorganism is selected from the group consisting of Staphylococcus xylosus, Staphylococcus hominis, Staphylococcus nepalensis, Staphylococcus warneri, Staphylococcus borealis, Staphylococcus petrasii, Staphylococcus epidermidis, Staphylococcus lugdunensis, Staphylococcus caprae, Staphylococcus saprophyticus, Staphylococcus capitis, Staphylococcus haemolyticus, Staphylococcus condiment, Micrococcus spp. And Corynebacterium spp.

In some embodiments, the beneficial microorganism is at least one microorganism, such as at least two microorganisms, such as at least three microorganisms, such as at least four microorganisms, such as at least five microorganisms, such as at least six microorganisms, such as at least seven microorganisms, such as at least eight microorganisms, such as at least nine microorganisms, such as at least ten microorganisms, such as at least eleven microorganisms, such as at least twelve microorganisms, such as at least 13 microorganisms, such as at least 14 microorganisms, such as at least 15 microorganisms, such as at least 25 microorganisms, such as all microorganisms selected from the group consisting of Staphylococcus xylosus, Staphylococcus hominis, Staphylococcus nepalensis, Staphylococcus warneri, Staphylococcus borealis, Staphylococcus petrasii, Staphylococcus epidermidis, Staphylococcus lugdunensis, Staphylococcus caprae, Staphylococcus saprophyticus, Staphylococcus capitis, Staphylococcus haemolyticus, Staphylococcus condiment, Micrococcus spp. And Corynebacterium spp.

### Scars

A scar or scar tissue is an area of fibrous tissue that replaces normal skin after injury, such as after a wound. Many wounds, in particular wounds where the dermis is damaged, result in scarring. Generally, improving wound healing may decrease scar formation or prevent scars from being formed.

Scars may be treated in any individual in need thereof using the peptides disclosed herein, such as in any individual suffering from a disease or condition characterized by scar formation. The individual may for example suffer a disease or condition characterized by blistering, as described herein in the section “Disease or condition characterized by blistering”.

In some embodiments, however, the individual suffers from a skin disease or skin condition selected from the group consisting of burn wounds, chronic skin ulcers, infected acute wounds, pressure ulcers and chronic wounds such as venous ulcers, for example venous leg ulcers, or diabetic leg ulcers, wherein said wounds are characterized by scar formation. In some embodiments, the individual suffers from an infected wound, such as a wound infected with pathogenic bacteria, for example S. aureus.

In some embodiments, treatment is preventative.

In some embodiments, treatment reduces scar formation.

In some embodiments, treatment reduces the size of an existing scar.

In some embodiments, the scar is a fibrotic scar.

In some embodiments, treatment reduces the size of the scar by at least 10%, such as at least 20%, such as at least 30%, such as at least 35%, such as at least 40%, such as at least 45%, such as at least 50%, such as at least 60%, such as at least 70%, such as at least 80%, such as at least 90%, such as wherein treatment reduces scar formation by at least 95%.

In some embodiments, said reduction is as compared to the size of a scar formed from a wound of similar size and character which was not treated.

In some embodiments, treatment reduces the size of the scar by between 10% and 95%, such as between 10% and 90%, such as between 15% and 80%, such as between 10% and 40%, such as between 30% and 80%, such as between 50% and 90%, such as between 20% and 50%, such as between 40% and 60%, such as between 10% and 25%, such as between 30% and 70%, such as between 80% and 90%, such as between 70% and 80% such as between 50% and 70% such as between 20% and 70%.

### Peptides

The invention relates to compounds comprising peptides for use in methods of treatment of wounds and blisters in patients suffering from a skin disease or skin condition selected from the group consisting of blistering disease, a disease or condition characterized by excessive wound exudate, a disease or condition characterised by a dysregulated skin microbiome and scars.

The invention further relates to compounds comprising peptides for use in methods of treatment of a skin disease or skin condition in an individual in need thereof, wherein the skin disease or skin condition is a blistering disease or a wound, and wherein the treatment reduces the amount of fluid that exudes from one or more blisters or wounds.

The invention further relates to compounds comprising peptides for use in methods for promoting a healthy skin microbiome in an individual in need thereof.

The compound may thus comprise a peptide consisting of any of below mentioned amino acids, wherein the peptide has been further derivatised or conjugated to one or more additional moieties. The skilled person will appreciate that in a compound comprising a peptide consisting of any of below mentioned amino acids, one or more hydrogen moieties of said peptide may be substituted with a conjugated moiety.

The peptides may be thrombin-derived C-terminal (TCP) peptides.

In some embodiments, the compound comprises a peptide comprising or consisting of the amino acid sequence

X₁—X₂—X₃—X₄—X₅—X₆—W—X₈—X₉—X₁₀,

wherein
   X_{4, 6, 9} is any standard amino acid,
   X₁ is I, L or V,
   X₂ is any standard amino acid except C,
   X₃ is A, E, Q, R or Y,
   X₅ is any standard amino acid except R,
   X₈ is I or L,
   X₁₀ is any standard amino acid except H,
   wherein said peptide has a length of from 10 to 100 amino acid residues.

In some embodiments, the peptide comprises or consists of the amino acid sequence

X1—X2—X3—X4—X5—X6—W—X8—X9—X10—X11—X12—X13,

wherein
   X4, 6, 9, 11 is any standard amino acid,
   X1, is I, L or V
   X2 is any standard amino acid except C
   X3 is A, E, Q, R or Y
   X5 is any standard amino acid except R
   X8 is I or L
   X10 is any standard amino acid except H
   X12 is I, M or T
   X13 is D, K, Q or R
      and wherein said peptide has a length of from 20 to 100 amino acid residues.

In some embodiments, the peptide comprises or consists of the amino acid sequence

X1—X2—X3—X4—X5—X6—W—X8—X9—X10—X11—X12—X13—X14—X15—X16—X17,

wherein
X4, 6, 9, 11, 14, 15 is any standard amino acid
X1 is I, L or V
X2 is any standard amino acid except C
X3 is A, E, Q, R or Y
X5 is any standard amino acid except R
X8 is I or L
X10 is any standard amino acid except H
X12 is I, M or T
X13 is D, K, Q or R
X16 is G or D
X17 is E, L, G, R or K
   and wherein said peptide has a length of from 20 to 100 amino acid residues.

In some embodiments, the peptide has a length of at least 10 amino acids, such as at least 12 amino acids, such as at least 14 amino acids, such as at least 16 amino acids, such as at least 18 amino acids, such as at least 20 amino acids, such as at least 25 amino acids, such as at least 30 amino acids, such as at least 40 amino acids, such as at least 50 amino acids, such as at least 60 amino acids, such as at least 70 amino acids, such as at least 80 amino acids, such as wherein the peptide has a length of at least 90 amino acids.

In some embodiments, the peptide has a length of between 10 and 50 amino acids, such as between 10 and 40 amino acids, such as between 10 and 30 amino acids, such as between 10 and 25 amino acids, such as between 12 and 25 amino acids, such as between 15 and 25 amino acids, such as between 20 and 40 amino acids, such as between 20 and 30 amino acids, such as between 10 and 35 amino acids, such as between 10 and 25 amino acids, such as between 13 and 23 amino acids, such as between 18 and 25 amino acids.

In some embodiments, the peptide comprises or consists of any of the amino acid sequences:

```
  (SEQ ID NO: 1)
GKYGFYTHVFRLKKWIQKVIDQFGE,
  (SEQ ID NO: 2)
FYTHVFRLKKWIQKVIDQFGE,
  (SEQ ID NO: 3)
GKYGFYTHVFRLKKWIQKVI,
  (SEQ ID NO: 4)
HVFRLKKWIQKVIDQFGE,
  (SEQ ID NO: 5)
KYGFYTHVFRLKKWIQKVIDQFGE,
  (SEQ ID NO: 6)
GKYGFYTHVFRLKKWIQKVIDQF,
  (SEQ ID NO: 7)
GKYGFYTHVFRLKKWIQKV, or
  (SEQ ID NO: 8)
KKWIQKVIDQFGE;
```

wherein up to 2 amino acids of said sequences may be substituted for another amino acid.

In some embodiments, the peptide comprises or consists of any of the amino acid sequences:

(SEQ ID NO: 1)
GKYGFYTHVFRLKKWIQKVIDQFGE,
(SEQ ID NO: 2)
FYTHVFRLKKWIQKVIDQFGE,
(SEQ ID NO: 3)
GKYGFYTHVFRLKKWIQKVI,
(SEQ ID NO: 4)
HVFRLKKWIQKVIDQFGE,
(SEQ ID NO: 5)
KYGFYTHVFRLKKWIQKVIDQFGE,
(SEQ ID NO: 6)
GKYGFYTHVFRLKKWIQKVIDQF,
(SEQ ID NO: 7)
GKYGFYTHVFRLKKWIQKV,
or
(SEQ ID NO: 8)
KKWIQKVIDQFGE;

In some embodiments, the peptide comprises or consists of any of the amino acid sequences:

(SEQ ID NO: 1)
GKYGFYTHVFRLKKWIQKVIDQFGE,
or
(SEQ ID NO: 3)
GKYGFYTHVFRLKKWIQKVI

In some embodiments, the peptide has at least 80% sequence identity, such as at least 85% sequence identity, such as at least 90% sequence identity, such as at least 95% sequence identity, such as at least 99% sequence identity with the amino acid sequence:

(SEQ ID NO: 1)
GKYGFYTHVFRLKKWIQKVIDQFGE
(SEQ ID NO: 2)
FYTHVFRLKKWIQKVIDQFGE,
(SEQ ID NO: 3)
GKYGFYTHVFRLKKWIQKVI,
(SEQ ID NO: 4)
HVFRLKKWIQKVIDQFGE,
(SEQ ID NO: 5)
KYGFYTHVFRLKKWIQKVIDQFGE,
(SEQ ID NO: 6)
GKYGFYTHVFRLKKWIQKVIDQF,
(SEQ ID NO: 7)
GKYGFYTHVFRLKKWIQKV,
or
(SEQ ID NO: 8)
KKWIQKVIDQFGE.

In some embodiments, the peptide consists of the amino acid sequence:

(SEQ ID NO: 1)
GKYGFYTHVFRLKKWIQKVIDQFGE.

In some embodiments, the peptide comprises or consists of one of the amino acids sequences selected from the group consisting of SEQ ID NO: 1, SEQ ID NO: 2, SEQ ID NO: 3, SEQ ID NO: 4, SEQ ID NO: 5, SEQ ID NO: 6 and SEQ ID NO: 7, wherein any of said sequences comprises one amino acid substitution, such as two amino acid substitutions, such as three amino acid substitutions, such as four amino acid substitutions, such as five amino acid substitutions.

In some embodiments, the amino acid substitution is a conservative substitution.

In some embodiments, the compound disclosed herein may consist of the peptides disclosed herein above. However, in other embodiments, the compound may comprise a peptide conjugated to one or more additional moieties. Thus, the peptide of the present invention may be modified or derivatised in any way suitable. In other words, any of the amino acids comprised in the peptides may be modified or derivatised. In some embodiments, one or more of the amino acids comprised in the peptide are PEGylated, esterified, amidated, acylated, acetylated, alkenylated and/or alkylated.

Further, the peptide may be modified or derivatised as described in international patent application WO2011/036442 on p. 11, I. 1 to p. 15, I. 14.

Thus, in some embodiments, one of more amino acids of the peptide is PEGylated, amidated, acylated, acetylated, alkenylated and/or alkylated. Thus, the peptide may consist of the sequence X1-X2-X3-X4-X5-X6-W-X8-X9-X10, wherein each X is as defined herein above, except that one of more amino acids may be PEGylated, amidated, acylated, acetylated, alkenylated and/or alkylated.

The compound may also be a pharmaceutically acceptable acid or base addition salt of the peptide. The acids which are used to prepare the pharmaceutically acceptable acid addition salts of the peptides are those which form non-toxic acid addition salts, i.e. salts containing pharmacologically acceptable anions, such as the hydrochloride, hydrobromide, hydroiodide, nitrate, sulphate, bisulphate, acid, acetate, lactate, citrate, acid citrate, tartrate, bitartrate, succinate, maleate, fumarate, gluconate, saccharate, benzoate, methanesulphonate, ethanesulphonate, benzenesulphonate, p-toluenesulphonate and pamoate [i.e. 1,1′-methylene-bis-(2-hydroxy-3 naphthoate)] salts, among others.

### Individual

The present invention relates to a compound for use in methods of treatment of a skin disease or skin condition is selected from the group consisting of a disease or condition characterized by blistering, a disease or condition characterized by excessive wound exudate, a disease or condition characterised by a dysregulated skin microbiome and scars, in an individual in need thereof.

The present invention further relates to a compound for use in a method of treatment of a skin disease or skin condition in an individual in need thereof, wherein the skin disease or skin condition is a wound or a disease or condition characterized by blistering, and wherein the treatment reduces the amount of fluid that exudes from one or more wound or blister.

The present invention also a compound for use in a method for promoting a healthy skin microbiome in an individual in need thereof.

Preferably, the individual is a human being.

In some embodiments, the individual is a child, such as a human being of not more than 18 years of age.

As described herein, the individual may suffer from one or more skin conditions or skin diseases selected from the group consisting of a disease or condition characterized by blistering, a disease or condition characterized by excessive wound exudate, a disease or condition characterised by a dysregulated skin microbiome and scars.

In one embodiment, the individual suffers from a disease or condition characterized by blistering. Commonly, blistering diseases result in blisters that develop into wounds.

In one embodiment, the disease or condition characterized by blistering is selected from the group consisting of:
epidermolysis bullosa (EB),
burn-induced blisters,
chilblains,
cryotherapy-induced blisters,
virus-induced blisters, such as chickenpox, shingles, herpes simplex, atypical enterovirus infection, hand, foot and mouth disease, eczema herpeticum, bullous impetigo and staphylococcal scalded skin syndrome,
pemphigus, such as pemphigus foliaceous, pemphigus vulgaris, paraneoplastic pemphigus, pemphigus erythematosus or pemphigus vegetans,
pemphigoid, such as bullous pemphigoid, cicatricial pemphigoid, 200 k pemphigoid or mucous membrane pemphigoid,
linear IgA bullous dermatosis,
dermatitis herpetiformis,
Porphyria cutanea tarda,
bullous lupus erythematosus,
Hailey-Hailey disease and
Steven Johnson syndrome (toxic epidermal necrolysis).

In one embodiment, the individual suffers from EB. In some embodiments, the individual suffers from is inherited EB or acquired EB. In some embodiments, the individual suffers from epidermolysis bullosa simplex, junctional epidermolysis bullosa, dystrophic epidermolysis bullosa, dominant dystrophic epidermolysis bullosa, recessive dystrophic epidermolysis bullosa, Kindler syndrome or epidermolysis bullosa acquisita.

In one embodiment, the individual suffers from burn-induced blisters.

In one embodiment, the individual suffers from a disease or condition characterized by excessive wound exudate.

In some embodiments, said disease or condition characterized by excessive wound exudate is selected from the group consisting of burn wounds, chronic skin ulcers, infected acute wounds, pressure ulcers and chronic wounds such as venous ulcers, for example venous leg ulcers, or diabetic leg ulcers.

In some embodiments, the disease or condition characterized by excessive wound exudate is a disease or condition characterized by blistering. For example, the disease or condition characterized by excessive wound exudate may be EB and/or burn-induced blisters as defined herein above.

In some embodiments, the individual suffers from a disease or condition characterized by dysregulated skin microbiome.

In some embodiments, said disease or condition characterized by dysregulated skin microbiome is selected from the group consisting of burn wounds, chronic skin ulcers, infected acute wounds, pressure ulcers and chronic wounds such as venous ulcers, for example venous leg ulcers, or diabetic leg ulcers.

In some embodiments, the disease or condition characterized by dysregulated skin microbiome is a disease or condition characterized by blistering. For example, the disease or condition characterized by a dysregulated skin microbiome may be EB.

In some embodiments, the individual suffers from scars or a disease or condition characterized by or resulting in scarring of the skin.

In some embodiments, said disease or condition characterized by or resulting in scarring of the skin is selected from the group consisting of burn wounds, chronic skin ulcers, infected acute wounds, pressure ulcers and chronic wounds such as venous ulcers, for example venous leg ulcers, or diabetic leg ulcers.

In some embodiments, the disease or condition characterized by or resulting in scarring of the skin is a disease or condition characterized by blistering. For example, the disease or condition characterized by or resulting in scarring of the skin may be EB.

### Administration

The invention provides compounds comprising peptides for use in methods of treatment. The treatment may be preventative, curative and/or ameliorating treatment, preferably curative and/or ameliorating treatment.

The compounds comprising peptides may be administered to the individual in need thereof by any suitable route of administration. For example, the compound may be administered systemically or by local administration directly to the local site affected by the disorder or condition.

Preferably, the compound is administered topically. For example, the compound is administered to one or more areas of the individual affected by the skin condition or skin disorder.

In some embodiments, the compound is comprised in a product. For example, the compound may be comprised in a patch, a dry patch, a plaster, a bandage, a film or a dressing.

The compound may be formulated in any way suitable for the route of administration. In some embodiments, the compound is formulated in an ointment, cream, emulsion, lotion, powder, spray, solution, viscous solution or gel, such as a hydrogel. Preferably, the compound is formulated in a hydrogel. In some embodiments the formulated compound is comprised in a patch, a dry patch, a plaster, a bandage, a film or a dressing.

The skilled person will be able to formulate the compounds of the invention in a manner suitable for the route of administration chosen. For a brief review of present methods useful for drug delivery, see, Langer, Science 249:1527-1533 (1990). Methods for preparing administrable compounds are known or are apparent to those skilled in the art and are described in more detail in, for example, Remington's Pharmaceutical Science, 17^{th} Ed., Mack Publishing Company, Easton, Pa. (1985), and which is hereinafter referred to as “Remington”.

In one embodiment, the compound is formulated as described in international patent application WO 2021/121843.

Preferably, the compound is administered in a therapeutically effective amount. Even more preferably, the compound is administered at an effective dose, i.e. a therapeutically effective amount, periodically at an administration interval such that at least one symptom or feature of the skin disorder or skin condition is reduced in intensity, severity, duration or frequency.

### Composition

The compound may be formulated in any way suitable, such as in any composition suitable for treatment of the individual in need thereof. Thus, in some embodiments, the compound is formulated in a composition. In some embodiments, the composition further comprises one or more pharmaceutically acceptable carrier.

In some embodiments, the composition is comprised in a product. For example, the compound may be comprised in a patch, a dry patch, a plaster, a bandage, a film or a dressing.

In some embodiments, the concentration of the compound in the composition is between 0.5 and 50 mg/mL, such as between 0.8 and 10 mg/mL, such as between 0.8 and 3 mg/mL, such as between 2.5 and 9 mg/mL, such as between 0.86 and 8.6 mg/mL, such as between 2.9 and 8.6 mg/mL.

In some embodiments, the concentration of the peptide or compound in the composition is 0.86 mg/mL, 2.9 mg/ml or 8.6 mg/mL.

In some embodiments, the concentration of the compound in the composition is between 0.05 wt % and 5 wt %, such as between 0.08 wt % and 1 wt %, such as between 0.08 wt % and 0.3 wt %, such as between 0.25 wt % and 0.9 wt %, such as between 0.086 wt % and 0.86 wt %, such as between 0.29 wt % and 0.86 wt %.

In some embodiments, the concentration of the peptide or compound in the composition is 0.086 wt %, 0.29 wt % or 0.86 wt %.

Preferably, the composition is administered in a therapeutically effective amount.

In some embodiments, between 0.075 mg/cm² and 600 mg/cm² of the compound is administered to the individual affected by the skin condition or skin disorder, such as between 0.1 mg/cm² and 1.5 mg/cm².

In some embodiments, 0.145 mg/cm², 0.29 mg/cm², 0.43 mg/cm² or 0.86 mg/cm² of the peptide or compound is administered to the individual affected by the skin condition or skin disorder.

In some embodiments, the composition, compound or peptide is administered for at least one week, such as at least two weeks, such as at least three weeks, such as at least four weeks, such as at least five weeks, such as at least six weeks, such as at least seven weeks, such as at least eight weeks, such as at least ten weeks, such as at least twelve weeks, or more.

In some embodiments, the composition, compound or peptide is administered for between one week and twelve weeks, such as between two weeks and eight weeks.

In some embodiments, the composition, compound or peptide is administered for at least one month, such as at least two months, such as at least three months, such as at least four months, such as at least five months, such as at least six months, such as at least seven months, such as at least eight months, such as at least ten months, such as at least one year, such as at least two years.

In some embodiments, the composition, compound or peptide is administered for between one month and two years, such as between one month and one year, such as between one month and six months, such as between one month and two months.

In some embodiments, the peptide or compound is administered for two weeks at a concentration of between 2.5 and 3.5 mg/mL, such as 2.9 mg/mL, followed by administration for two weeks at a concentration of between 8 and 9 mg/mL, such as 8.6 mg/mL.

In some embodiments, the composition, compound or peptide is administered three times per week, such as every second or third day for at least four weeks.

In some embodiments, the peptide or compound is administered at an increasing dose or concentration over time, such as for two, three, four or more periods wherein the dose or concentration of peptide or compound is increased for each period.

In some embodiments:
a. between 0.025 and 0.075 mL/cm², such as 0.05 mL/cm², of the peptide or compound is administered to the individual affected by the skin condition or skin disorder during the first period of treatment, wherein the concentration of said peptide or compound is between 2.5 and 3.5 mg/mL, such as 2.9 mg/mL;
b. between 0.05 and 0.15, L/cm², such as 0.1 mL/cm², of the peptide or compound is administered to the individual affected by the skin condition or skin during the second period of treatment, wherein the concentration of said peptide or compound is between 2.5 and 3.5 mg/mL, such as 2.9 mg/mL;
c. between 0.025 and 0.075 mL/cm², such as 0.05 mL/cm², of the peptide or compound is administered to the individual affected by the skin condition or skin during the third period of treatment, wherein the concentration of said peptide or compound is between 8 and 9 mg/mL, such as 8.6 mg/mL; and
d. between 0.05 and 0.15, L/cm², such as 0.1 mL/cm², of the peptide or compound is administered to the individual affected by the skin condition or skin during the fourth period of treatment, wherein the concentration of said peptide or compound is between 8 and 9 mg/ml, such as 8.6 mg/mL;
   optionally wherein each period of treatment is approximately one week, optionally wherein the compound or peptide is administered three times per week, such as every second or third day.

In some embodiments:
a. between 0.1 and 0.2 mg/cm², such as 0.14 mg/cm², of the peptide or compound is administered to the individual affected by the skin condition or skin disorder during the first period of treatment;
b. between 0.25 and 0.35 mg/cm², such as 0.29 mg/cm², of the peptide or compound is administered to the individual affected by the skin condition or skin during the second period of treatment;
c. between 0.4 and 0.5 mg/cm², such as 0.43 mg/cm², of the peptide or compound is administered to the individual affected by the skin condition or skin during the third period of treatment; and
d. between 0.8 and 0.9 mg/cm², such as 0.86 mg/cm², of the peptide or compound is administered to the individual affected by the skin condition or skin during the fourth period of treatment;
   optionally wherein each period of treatment is approximately one week,
   optionally wherein the compound or peptide is administered three times per week, such as every second or third day.

In some embodiments, the first, second, third and fourth period of treatment is between 2 days and 2 months, such as between 4 days and 1 month, such as between 6 days and 3 weeks, such as between one week and two weeks.

The composition may further comprise any components suitable.

In one embodiment, the composition is a hydrogel comprising EDTA.

In some embodiments, the composition further comprises:
a. EDTA, and
b. an aqueous buffer,
   wherein
      i. the composition has a pH of at the most 8 and/or
      ii. the concentration of the compound in the composition is at least 0.08 wt %.

In other embodiments, the composition further comprises:
a. a polymer capable of forming a hydrogel when mixed with an aqueous solution, and
b. an aqueous solution
   wherein
      i. the concentration of the compound in the composition is at least 0.08 wt % and/or
      ii. the polymer is present in said composition at a concentration of at least 0.05 wt %.

### Items

1. A compound for use in a method of treating a skin disease or skin condition in an individual in need thereof, wherein said skin disease or skin condition is selected from the group consisting of a disease or condition characterized by blistering, a disease or condition characterized by excessive wound exudate, a disease or condition characterised by a dysregulated skin microbiome and scars,
   wherein the compound comprises a peptide comprising or consisting of the amino acid sequence

X₁-X₂-X₃-X₄-X₅-X₆-W-X₈-X₉-X₁₀,

wherein
X_{4, 6, 9} is any standard amino acid,
X₁ is I, L or V,
X₂ is any standard amino acid except C,
X₃ is A, E, Q, R or Y,
X₅ is any standard amino acid except R,
X₈ is I or L,
X₁₀ is any standard amino acid except H,
wherein said peptide has a length of from 10 to 100 amino acid residues.
2. A method for treatment of a skin disease or skin condition in an individual in need thereof, wherein said skin disease or skin condition is selected from the group consisting of a disease or condition characterized by blistering, a disease or condition characterized by excessive wound exudate, a disease or condition characterised by a dysregulated skin microbiome and scars,
   said method comprising administering a compound comprising a peptide comprising or consisting of the amino acid sequence

X₁-X₂-X₃-X₄-X₅-X₆-W-X₈-X₉-X₁₀,

wherein
X_{4, 6, 9} is any standard amino acid,
X₁ is I, L or V,
X₂ is any standard amino acid except C,
X₃ is A, E, Q, R or Y,
X₅ is any standard amino acid except R,
X₈ is I or L,
X₁₀ is any standard amino acid except H,
wherein said peptide has a length of from 10 to 100 amino acid residues.
3. A pharmaceutical composition for use in the treatment of a skin disease or skin condition in an individual in need thereof, wherein said skin disease or skin condition is selected from the group consisting of a disease or condition characterized by blistering, a disease or condition characterized by excessive wound exudate, a disease or condition characterised by a dysregulated skin microbiome and scars,
   wherein the composition comprises a compound comprising a peptide comprising or consisting of the amino acid sequence

X₁-X₂-X₃-X₄-X₅-X₆-W-X₈-X₉-X₁₀,

wherein
X_{4, 6, 9} is any standard amino acid,
X₁ is I, L or V,
X₂ is any standard amino acid except C,
X₃ is A, E, Q, R or Y,
X₅ is any standard amino acid except R,
X₈ is I or L,
X₁₀ is any standard amino acid except H,
wherein said peptide has a length of from 10 to 100 amino acid residues.
4. Use of a compound in the manufacture of a medicament for the treatment of a skin disease or skin condition in an individual in need thereof, wherein said skin disease or skin condition is selected from the group consisting of a disease or condition characterized by blistering, a disease or condition characterized by excessive wound exudate, a disease or condition characterised by a dysregulated skin microbiome and scars,
   wherein the compound comprises a peptide comprising or consisting of the amino acid sequence

X₁-X₂-X₃-X₄-X₅-X₆-W-X₈-X₉-X₁₀,

wherein
X_{4, 6, 9} is any standard amino acid,
X₁ is I, L or V,
X₂ is any standard amino acid except C,
X₃ is A, E, Q, R or Y,
X₅ is any standard amino acid except R,
X₈ is I or L,
X₁₀ is any standard amino acid except H,
wherein said peptide has a length of from 10 to 100 amino acid residues.
5. A compound for use in a method of reducing the amount of fluid that exudes from one or more wound or blister in an individual in need thereof,
   wherein the compound comprises a peptide comprising or consisting of the amino acid sequence

X₁-X₂-X₃-X₄-X₅-X₆-W-X₈-X₉-X₁₀,

wherein
X_{4, 6, 9} is any standard amino acid,
X₁ is I, L or V,
X₂ is any standard amino acid except C,
X₃ is A, E, Q, R or Y,
X₅ is any standard amino acid except R,
X₈ is I or L,
X₁₀ is any standard amino acid except H,
wherein said peptide has a length of from 10 to 100 amino acid residues.
6. A method of reducing the amount of fluid that exudes from one or more wound or blister in an individual in need thereof,
   said method comprising administering a compound comprising a peptide comprising or consisting of the amino acid sequence

X₁-X₂-X₃-X₄-X₅-X₆-W-X₈-X₉-X₁₀,

wherein
X_{4, 6, 9} is any standard amino acid,
X₁ is I, L or V,
X₂ is any standard amino acid except C,
X₃ is A, E, Q, R or Y,
X₅ is any standard amino acid except R,
X₈ is I or L,
X₁₀ is any standard amino acid except H,
wherein said peptide has a length of from 10 to 100 amino acid residues.
7. A compound for use in a method of treatment of a skin disease or skin condition in an individual in need thereof, wherein the skin disease or skin condition is a wound or a disease or condition characterized by blistering, and wherein the treatment reduces the amount of fluid that exudes from one or more wound or blister, and wherein the compound comprises a peptide comprising or consisting of the amino acid sequence

X₁-X₂-X₃-X₄-X₅-X₆-W-X₈-X₉-X₁₀,

wherein
X_{4, 6, 9} is any standard amino acid,
X₁ is I, L or V,
X₂ is any standard amino acid except C,
X₃ is A, E, Q, R or Y,
X₅ is any standard amino acid except R,
X₈ is I or L,
X₁₀ is any standard amino acid except H,
wherein said peptide has a length of from 10 to 100 amino acid residues.
8. A method of treatment of a skin disease or skin condition in an individual in need thereof, wherein the skin disease or skin condition is a wound or a disease or condition characterized by blistering, and wherein the treatment reduces the amount of fluid that exudes from one or more wound or blister, said method comprising administering a compound comprising a peptide comprising or consisting of the amino acid sequence

X₁-X₂-X₃-X₄-X₅-X₆-W-X₈-X₉-X₁₀,

wherein
X_{4, 6, 9} is any standard amino acid,
X₁ is I, L or V,
X₂ is any standard amino acid except C,
X₃ is A, E, Q, R or Y,
X₅ is any standard amino acid except R,
X₈ is I or L,
X₁₀ is any standard amino acid except H,
wherein said peptide has a length of from 10 to 100 amino acid residues.
9. A compound for promoting a healthy skin microbiome in an individual in need thereof,
   wherein the compound comprises a peptide comprising or consisting of the amino acid sequence

X₁-X₂-X₃-X₄-X₅-X₆-W-X₈-X₉-X₁₀,

wherein
X_{4, 6, 9} is any standard amino acid,
X₁ is I, L or V,
X₂ is any standard amino acid except C,
X₃ is A, E, Q, R or Y,
X₅ is any standard amino acid except R,
X₈ is I or L,
X₁₀ is any standard amino acid except H,
wherein said peptide has a length of from 10 to 100 amino acid residues.
10. A method for promoting a healthy skin microbiome in an individual in need thereof,
   said method comprising administering a compound comprising a peptide comprising or consisting of the amino acid sequence

X₁-X₂-X₃-X₄-X₅-X₆-W-X₈-X₉-X₁₀,

wherein
X_{4, 6, 9} is any standard amino acid,
X₁ is I, L or V,
X₂ is any standard amino acid except C,
X₃ is A, E, Q, R or Y,
X₅ is any standard amino acid except R,
X₈ is I or L,
X₁₀ is any standard amino acid except H,
wherein said peptide has a length of from 10 to 100 amino acid residues.
11. The compound for use, the method, the pharmaceutical composition or the use according to any one of the preceding items, wherein the individual suffers from a skin disease or skin condition selected from the group consisting of a disease or condition characterized by blistering, a disease or condition characterized by excessive wound exudate, a disease or condition characterised by a dysregulated skin microbiome and scars.
12. The compound for use, the method, the pharmaceutical composition or the use according to any one of the preceding items, wherein the individual suffers from a skin disease or skin condition selected from the group consisting of burn wounds, chronic skin ulcers, infected acute wounds, pressure ulcers and chronic wounds such as venous ulcers, for example venous leg ulcers, and diabetic leg ulcers.
13. The compound for use, the method, the pharmaceutical composition or the use according to any one of the preceding items, wherein the individual suffers from eczema, such as of atopic dermatitis.
14. The compound for use, the method, the pharmaceutical composition or the use according to any one of the preceding items, wherein the disease or condition characterized by blistering is selected from the group consisting of:
   epidermolysis bullosa (EB),
   burn-induced blisters,
   chilblains,
   cryotherapy-induced blisters,
   virus-induced blisters, such as chickenpox, shingles, herpes simplex, atypical enterovirus infection, hand, foot and mouth disease, eczema herpeticum, bullous impetigo and staphylococcal scalded skin syndrome,
   pemphigus, such as pemphigus foliaceous, pemphigus vulgaris, paraneoplastic pemphigus, pemphigus erythematosus or pemphigus vegetans,
   pemphigoid, such as bullous pemphigoid, cicatricial pemphigoid, 200 k pemphigoid or mucous membrane pemphigoid,
   linear IgA bullous dermatosis,
   dermatitis herpetiformis,
   Porphyria cutanea tarda,
   bullous lupus erythematosus,
   Hailey-Hailey disease and
   Steven Johnson syndrome (toxic epidermal necrolysis).
15. The compound for use, the method, the pharmaceutical composition or use according to any one of the preceding items, wherein the disease or condition characterized by blistering is epidermolysis bullosa (EB).
16. The compound for use, the method, the pharmaceutical composition or the use according to any one of the preceding items, wherein the disease or condition characterized by blistering is EB, and wherein EB is inherited epidermolysis bullosa or acquired epidermolysis bullosa.
17. The compound for use, the method, the pharmaceutical composition or the use according to any one of the preceding items, wherein the disease or condition characterized by blistering is EB, wherein EB is epidermolysis bullosa simplex, junctional epidermolysis bullosa, dystrophic epidermolysis bullosa, such as dominant dystrophic epidermolysis bullosa or recessive dystrophic epidermolysis bullosa, Kindler syndrome or epidermolysis bullosa acquisita.
18. The compound for use, the method, the pharmaceutical composition or the use according to any one of the preceding items, wherein said treatment reduces the size of one or more blisters or wounds.
19. The compound for use, the method, the pharmaceutical composition or the use according to any one of the preceding items, wherein treatment reduces the size of one or more blister or wound by at least 10%, such as at least 20%, such as at least 30%, such as at least 35%, such as at least 40%, such as at least 45%, such as at least 50%, such as at least 60%, such as at least 70%, such as at least 80%, such as at least 90%, such as wherein the treatment reduces the size of one or more blister or wound by at least 95% as compared to the size of the one or more blister or wound before treatment, or as compared to the size of a blister or wound which was not treated with the compound comprising the peptide, and which before treatment was of similar size and character as the one or more treated blister or wound.
20. The compound for use, the method, the pharmaceutical composition or the use according to any one of the preceding items, wherein treatment reduces the size of one or more blister or wound by 100%, such that the treatment results in that one or more blister or wound heals completely.
21. The compound for use, the method, the pharmaceutical composition or the use according to any one of the preceding items, wherein the size of one or more blister or wound is reduced after treatment for at least two days, such as at least five days, such as at least one week, such as at least two weeks, such as at least three weeks, such as at least four weeks, such as at least one month, such as at least two months, such as at least five months, such as at least six months, such as at least seven months, such as at least eight months, such as at least nine months, such as at least ten months, such as at least eleven months, such as wherein the size of one or more blister or wound is reduced after one year of treatment.
22. The compound for use, the method, the pharmaceutical composition or the use according to any one of the preceding items, wherein the wound or blister stays healed and does not reopen for at least two days, such as at least five days, such as at least one week, such as at least two weeks, such as at least three weeks, such as at least four weeks, such as at least one month, such as at least two months, such as at least five months, such as at least six months, such as at least seven months, such as at least eight months, such as at least nine months, such as at least ten months, such as at least eleven months, such as at least one year, such as at least two years, such as at least three years, such as at least four years, such as at least five years after the treatment is completed, such as wherein the wound or blister stays healed and does reopen at all after the treatment is completed.
23. The compound for use, the method, the pharmaceutical composition or the use according to any one of the preceding items, wherein said treatment reduces the pain experienced by the individual, such as wherein said treatment reduces the pain of one or more wound or blister,
   optionally wherein the pain is measured using a pain scale selected from the group consisting of Neonatal/Infants Pain Scale (NIPS), Children's Hospital of Eastern Ontario Pain Scale (CHEOPS), Face, Legs, Activity, Crying, Consolability (FLACC) scale, Faces Pain Scale (Wong-Baker Faces Pain Rating Scale), Coloured Analogue Scale, Visual Analog Scale, Verbal Numerical Rating Scale and Wharton Impairment and Pain Scale.
24. The compound for use, the method, the pharmaceutical composition or the use according to any one of the preceding items, wherein the pain is reduced at least one step or score on a pain scale, such as at least two steps or scores, such as at least three steps or scores, such as at least four steps or scores, such as at least five steps or scores, such as at least six steps or scores, such as at least seven steps or scores, such as at least eight steps or scores, such as at least nine steps or scores, such as wherein the pain is reduced ten steps or scores on a pain scale,
   optionally wherein the pain is measured using a pain scale selected from the group consisting of Neonatal/Infants Pain Scale (NIPS), Children's Hospital of Eastern Ontario Pain Scale (CHEOPS), Face, Legs, Activity, Crying, Consolability (FLACC) scale, Faces Pain Scale (Wong-Baker Faces Pain Rating Scale), Coloured Analogue Scale, Visual Analog Scale, Verbal Numerical Rating Scale and Wharton Impairment and Pain Scale.
25. The compound for use, the method, the pharmaceutical composition or the use according to any one of the preceding items, wherein said treatment reduces the itch experienced by the individual, such as the itch of one or more wound or blister,
   optionally wherein the itch is evaluated via subjective patient reporting or by objective measurement of scratching activity or scratching induced skin changes,
   optionally wherein the itch is measured using the Leuven Itch Scale,
   optionally wherein the itch is measured using the 5-D itch scale.
26. The compound for use, the method, the pharmaceutical composition or the use according to any one of the preceding items, wherein treatment reduces the amount of fluid that exudes from one or more blister or wound, such as wherein treatment reduces the amount of wound exudate.
27. The compound for use, method of treatment, pharmaceutical composition or use according to any one of the preceding items, wherein treatment decreases the amount of fluid that exudes from a wound or blister by at least 10%, such as at least 20%, such as at least 30%, such as at least 35%, such as at least 40%, such as at least 45%, such as at least 50%, such as at least 60%, such as at least 70%, such as at least 80%, such as at least 90%, such as wherein the amount of wound exudate is decreased by at least 95%,
   optionally wherein the amount of wound exudate is estimated by visual inspection of the wound dressing,
   optionally wherein the amount of wound exudate is measured by imaging of the wound dressing,
   optionally wherein the amount of wound exudate is measured by weighing the wound dressing,
   optionally wherein the amount of wound exudate is measured by measuring the protein content in the wound dressing, such as the total protein content in the wound dressing, optionally wherein the protein content is measure using the Bradford protein assay,
   optionally wherein the decrease is as compared to before starting treatment of the wound or blister,
   optionally wherein the decrease is as compared to a wound or blister of similar size and character which was not treated with the compound comprising the peptide and which before treatment had a similar amount of wound exudate as the treated wound or blister.
28. The compound for use, method of treatment, pharmaceutical composition or use according to any one of the preceding items, wherein treatment decreases the amount of fluid that exudes from the wound or blister at least one step or score on an exudate scale, such as at least two steps or scores, such as at least three steps or scores, such as at least four steps or scores, such as at least five steps or scores on a wound exudate scale,
   optionally wherein the scoring or scaling of exudate is carried out by visually inspecting the wound dressing,
   optionally wherein the scoring or scaling of leakage is carried out using a visual scoring scale from 0-5, wherein 0 represents “No exudation in wound area” and 5 represents “Exudation soaking the dressing in an area exceeding the wound area”.
29. The compound for use, method of treatment, pharmaceutical composition or use according to any one of the preceding items, wherein treatment reduces the amount of heparin binding protein in the wound exudate,
   optionally wherein the amount of HBP is decreased at least 20%, such as at least 30%, such as at least 40%, such as at least 50%, such as at least 60%, such as at least 70%, such as at least 80%, such as at least 90%,
   optionally wherein the decrease is as compared to before starting treatment of the wound or blister,
   optionally wherein the decrease is as compared to a wound or blister of similar size and character which was not treated with the compound comprising the peptide and which before treatment had a similar amount of wound exudate as the treated wound or blister.
30. The compound for use, method of treatment, pharmaceutical composition or use according to any one of the preceding items, wherein treatment decreases the total protein content that exudes from a wound or blister.
31. The compound for use, method of treatment, pharmaceutical composition or use according to any one of the preceding items, wherein treatment reduces the amount of times that wound dressings need to be changed within a set time period, such as wherein treatment increases the amount of days passed before the wound dressings must be changed, such as wherein treatment results in wound dressings not needing to be changed as often as compared to before starting treatment,
   optionally wherein the timepoint for changing the wound dressing is determined based on the amount of exudate present in the dressing,
   optionally wherein the timepoint for changing the wound dressing is determined to reduce the risk of inflammation and/or infection.
32. The compound for use, method of treatment, pharmaceutical composition or use according to any one of the preceding items, wherein treatment increases the amount of days passed between changing wound dressings with half a day, such as with one day, such as with 1.5 days, such as with 2 days, such as with 2.5 days, such as with 3 days, such as with 3.5 days, such as with 4 days, such as with 4.5 days, such as with 5 days, such as wherein treatment increases the amount of days passed between changing wound dressings with 6 days,
   optionally wherein treatment results in that the wound dressing only needs to be changed every third day, such as every fourth day, such as every fifth day, such as every sixth day, such as wherein treatment results in that wound dressing only needs to be changed once per week.
33. The compound for use, the method, the pharmaceutical composition or the use according to any one of the preceding items, wherein treatment reduces the total amount of microorganisms present in one or more wound or blister, such as the total amount of bacteria present in one or more wound or blister.
34. The compound for use, the method, the pharmaceutical composition or the use according to any one of the preceding items, wherein treatment reduces the total amount of microorganisms present in one or more wound or blister by at least 10%, such as at least 20%, such as at least 30%, such as at least 35%, such as at least 40%, such as at least 45%, such as at least 50%, such as at least 60%, such as at least 70%, such as at least 80%, such as at least 90%, such as wherein the total amount of microorganisms present in one or more wound or blister is reduced by at least 95% as compared to the amount of bacteria present before treatment,
   optionally wherein the amount of microorganisms is measured by measuring the amount of colony-forming units (CFU) present in a wound dressing and/or in a wound or blister on the skin, wherein said measurement takes place before, during and/or after treatment.
35. The compound for use, the method, the pharmaceutical composition or the use according to any one of the preceding items, wherein the dysregulated skin microbiome is characterised by a decreased ratio of beneficial:harmful microorganisms as compared to the ratio of beneficial:harmful microorganisms in an individual not suffering from a dysregulated microbiome, such that the harmful microorganisms comprise a larger part of the total skin microbiome in the dysregulated skin microbiome as compared to the skin microbiome of an individual not suffering from a dysregulated skin microbiome.
36. The compound for use, the method, the pharmaceutical composition or the use according to any one of the preceding items, wherein the beneficial microorganisms are non-pathogenic microorganisms, such as non-pathogenic bacteria, and wherein the harmful microorganisms are pathogenic microorganisms, such as pathogenic bacteria.
37. The compound for use, the method, the pharmaceutical composition or the use according to any one of the preceding items, wherein treatment promotes a healthy skin microbiome.
38. The compound for use, the method, the pharmaceutical composition or the use according to any one of the preceding items, wherein treatment reduces the total amount of commensal skin microorganisms in the skin microbiome, such as wherein treatment reduces excessive bacterial load, for example wherein treatment reduces the overall bacterial load in a wound or blister.
39. The compound for use, the method, the pharmaceutical composition or the use according to any one of the preceding items, wherein treatment preserves the composition of the skin microbiome, such as wherein treatment preserves the microbial diversity of the skin microbiome.
40. The compound for use, the method, the pharmaceutical composition or the use according to any one of the preceding items, wherein treatment preserves the ratio between different microbial species and/or strains in the skin microbiome, such as wherein the ratio between different microbial species and/or strains in the skin microbiome is the same or similar before, during and/or after treatment.
41. The compound for use, the method, the pharmaceutical composition or the use according to any one of the preceding items, wherein treatment maintains the ratio of beneficial:harmful microorganisms in the skin microbiome, such as wherein the ratio of beneficial:harmful microorganisms in the skin microbiome is the same or similar before, during and/or after treatment,
   optionally wherein treatment increases the ratio of beneficial:harmful microorganisms in the skin microbiome, for example wherein treatment doubles, triples or quadruples the ratio of beneficial:harmful microorganisms in the skin microbiome, such that the beneficial microorganisms during and/or after treatment make up a larger part of the total skin microbiome relative to the harmful microorganisms.
42. The compound for use, method of treatment, pharmaceutical composition or use according to any one of the preceding items, wherein the harmful microorganism is selected from the group consisting of Staphylococcus aureus, Bacillus licheniformis, Bacillus cereus complex, Rothia amarae, Staphylococcus spp., Streptococcus spp., group A streptococci, such as Streptococcus pyogenes, and gram-negative bacteria, such as Pseudomonas aeruginosa, Proteobacteria spp. And Proteus spp.
43. The compound for use, the method, the pharmaceutical composition or the use according to any one of the preceding items, wherein the beneficial microorganism is selected from the group consisting of Staphylococcus xylosus, Staphylococcus hominis, Staphylococcus nepalensis, Staphylococcus warneri, Staphylococcus borealis, Staphylococcus petrasii, Staphylococcus epidermidis, Staphylococcus lugdunensis, Staphylococcus caprae, Staphylococcus saprophyticus, Staphylococcus capitis, Staphylococcus haemolyticus, Staphylococcus condiment, Corynebacterium spp. And Micrococcus spp.
44. The compound for use, the method, the pharmaceutical composition or the use according to any one of the preceding items, wherein treatment reduces the size of the scar.
45. The compound for use, the method, the pharmaceutical composition or use according to any one of the preceding items, wherein treatment reduces scar formation.
46. The compound for use, the method, the pharmaceutical composition or the use according to any one of the preceding items, wherein treatment reduces the size of the scar by at least 10%, such as at least 20%, such as at least 30%, such as at least 35%, such as at least 40%, such as at least 45%, such as at least 50%, such as at least 60%, such as at least 70%, such as at least 80%, such as at least 90%, such as wherein treatment reduces scar formation by at least 95% as compared to the size of a scar formed from a wound of similar size and character which was not treated.
47. The compound for use, the method, the pharmaceutical composition or the use according to any one of the preceding items, wherein the compound is administered topically in a therapeutically effective amount.
48. The compound for use, the method, the pharmaceutical composition or the use according to any one of the preceding items, wherein the compound is administered to one or more areas of the individual affected by the skin condition or skin disorder.
49. The compound for use, the method, the pharmaceutical composition or the use according to any one of the preceding items, wherein treatment is preventative, curative or ameliorating treatment.
50. The compound for use, the method, the pharmaceutical composition or the use according to any one of the preceding items, wherein the compound is comprised in a patch, a dry patch, a plaster, a bandage, a film or a dressing.
51. The compound for use, the method, the pharmaceutical composition or the use according to any one of the preceding items, wherein the compound is formulated in an ointment, cream, emulsion, lotion, powder, spray, solution, viscous solution or gel, such as a hydrogel, preferably wherein the compound is formulated in a hydrogel.
52. The compound for use, the method, the pharmaceutical composition or the use according to any one of the preceding items, wherein the compound is administered at an effective dose periodically at an administration interval such that at least one symptom or feature of the skin disorder or skin condition is reduced in intensity, severity, duration or frequency.
53. The compound for use, the method, the pharmaceutical composition or the use according to any one of the preceding items, wherein the individual is a human.
54. The compound for use, the method, the pharmaceutical composition or the use according to any one of the preceding items, wherein
   a. the peptide comprises or consists of the amino acid sequence

X1-X2-X3-X4-X5-X6-W-X8-X9-X10-X11-X12-X13,

wherein
   X4, 6, 9, 11 is any standard amino acid,
   X1, is I, L or V
   X2 is any standard amino acid except C
   X3 is A, E, Q, R or Y
   X5 is any standard amino acid except R
   X8 is I or L
   X10 is any standard amino acid except H
   X12 is I, M or T
   X13 is D, K, Q or R
   and wherein said peptide has a length of from 20 to 100 amino acid residues; or
b. the peptide comprises or consists of the amino acid sequence

X1-X2-X3-X4-X5-X6-W-X8-X9-X10-X11-X12-X13-
X14-X15-X16-X17,

wherein
X4, 6, 9, 11, 14, 15 is any standard amino acid
X1 is I, L or V
X2 is any standard amino acid except C
X3 is A, E, Q, R or Y
X5 is any standard amino acid except R
X8 is I or L
X10 is any standard amino acid except H
X12 is I, M or T
X13 is D, K, Q or R
X16 is G or D
X17 is E, L, G, R or K
and wherein said peptide has a length of from 20 to 100 amino acid residues.
55. The compound for use, the method, the pharmaceutical composition or the use according to any one of the preceding items, wherein the peptide has a length of 10 to 35 amino acids, such as 10 to 25 amino acids, such as 12 to 25 amino acids, such as 13 to 23 amino acids, such as 20 to 30 amino acids, preferably 15 to 25 amino acids.
56. The compound for use, the method, the pharmaceutical composition or the use according to any one of the preceding items, wherein the peptide comprises or consists of any of the amino acid sequences:

(SEQ ID NO: 1)
GKYGFYTHVFRLKKWIQKVIDQFGE,
(SEQ ID NO: 2)
FYTHVFRLKKWIQKVIDQFGE,
(SEQ ID NO: 3)
GKYGFYTHVFRLKKWIQKVI,
(SEQ ID NO: 4)
HVFRLKKWIQKVIDQFGE,
(SEQ ID NO: 5)
KYGFYTHVFRLKKWIQKVIDQFGE,
(SEQ ID NO: 6)
GKYGFYTHVFRLKKWIQKVIDQF,
(SEQ ID NO: 7)
GKYGFYTHVFRLKKWIQKV,
or
(SEQ ID NO: 8)
KKWIQKVIDQFGE;

preferably wherein the peptide comprises or consists of any of the amino acid sequences:

(SEQ ID NO: 1)
GKYGFYTHVFRLKKWIQKVIDQFGE,
or
(SEQ ID NO: 3)
GKYGFYTHVFRLKKWIQKVI;
or

wherein the peptide has at least 80% sequence identity, such as at least 85% sequence identity, such as at least 90% sequence identity, such as at least 95% sequence identity with any of the amino acid sequences:

(SEQ ID NO: 1)
GKYGFYTHVFRLKKWIQKVIDQFGE
(SEQ ID NO: 2)
FYTHVFRLKKWIQKVIDQFGE,
(SEQ ID NO: 3)
GKYGFYTHVFRLKKWIQKVI,
(SEQ ID NO: 4)
HVFRLKKWIQKVIDQFGE,
(SEQ ID NO: 5)
KYGFYTHVFRLKKWIQKVIDQFGE,
(SEQ ID NO: 6)
GKYGFYTHVFRLKKWIQKVIDQF,
(SEQ ID NO: 7)
GKYGFYTHVFRLKKWIQKV,
or
(SEQ ID NO: 8)
KKWIQKVIDQFGE;

preferably wherein the peptide consists of the amino acid sequence:

(SEQ ID NO: 1)
GKYGFYTHVFRLKKWIQKVIDQFGE.

57. The compound for use, the method, the pharmaceutical composition or the use according to any one of the preceding items, wherein one or more of the standard amino acids comprised in the peptide are modified or derivatised, optionally wherein one or more of the standard amino acids comprised in the peptide are PEGylated, amidated, acylated, acetylated, alkenylated and/or alkylated.
58. The compound for use, the method, the pharmaceutical composition or the use according to any one of the preceding items, wherein the compound is formulated in a composition.
59. The compound for use, the method, the pharmaceutical composition or the use according to any one of the preceding claims, wherein the composition is formulated as an ointment, cream, emulsion, lotion, powder, spray, solution, viscous solution or gel, such as a hydrogel, preferably wherein the compound is formulated in a hydrogel.
60. The compound for use, the method, the pharmaceutical composition or the use according to any one of the preceding items, wherein the composition further comprises one or more pharmaceutically acceptable carrier.
61. The compound for use, the method, the pharmaceutical composition or the use according to any one of the preceding items, wherein the composition is comprised in a patch, a dry patch, a plaster, a bandage, a film or a dressing.
62. The compound for use, the method, the pharmaceutical composition or the use according to any one of the preceding items, wherein the composition, compound or peptide is administered for at least one week, such as at least two weeks, such as at least three weeks, such as at least four weeks, such as at least five weeks, such as at least six weeks, such as at least seven weeks, such as at least eight weeks, such as at least ten weeks, such as at least twelve weeks, or more.
63. The compound for use, the method, the pharmaceutical composition or the use according to any one of the preceding items, wherein the composition, compound or peptide is administered for between one week and twelve weeks, such as between two weeks and eight weeks.
64. The compound for use, the method, the pharmaceutical composition or the use according to any one of the preceding items, wherein the composition, compound or peptide composition or peptide is administered for at least one month, such as at least two months, such as at least three months, such as at least four months, such as at least five months, such as at least six months, such as at least seven months, such as at least eight months, such as at least ten months, such as at least one year, such as at least two years.
65. The compound for use, the method, the pharmaceutical composition or the use according to any one of the preceding items, wherein the composition, compound or peptide is administered for between one month and two years, such as between one month and one year, such as between one month and six months, such as between one month and two months.
66. The compound for use, the method, the pharmaceutical composition or the use according to any one of the preceding items, wherein the concentration of the peptide or compound in said composition is between 0.5 and 50 mg/mL, such as between 0.8 and 10 mg/mL, such as between 0.8 and 3 mg/mL, such as between 2.5 and 9 mg/mL.
67. The compound for use, the method, the pharmaceutical composition or the use according to any one of the preceding items, wherein the concentration of the peptide or compound in said composition is 0.86 mg/mL, 2.9 mg/ml or 8.6 mg/mL.
68. The compound for use, the method, the pharmaceutical composition or the use according to any one of the preceding items, wherein the composition, compound or peptide is administered three times per week, such as every second or third day, optionally for at least four weeks.
69. The compound for use, the method, the pharmaceutical composition or the use according to any one of the preceding items, wherein between 0.075 mg/cm² and 600 mg/cm² of the peptide or compound is administered to the individual affected by the skin condition or skin disorder, such as between 0.1 mg/cm² and 1.5 mg/cm².
70. The compound for use, the method, the pharmaceutical composition or the use according to any one of the preceding items, wherein the peptide or compound is administered at an increasing dose or concentration over time, such as for two, three, four or more periods wherein the dose or concentration of peptide or compound is increased for each period.
71. The compound for use, the method, the pharmaceutical composition or the use according to any one of the preceding items, wherein the first, second, third and fourth period of treatment is between 2 days and 2 months, such as between 4 days and 1 month, such as between 6 days and 3 weeks, such as between one week and two weeks.
72. The compound for use, the method, the pharmaceutical composition or the use according to any one of the preceding items, wherein the peptide or compound is administered for two weeks at a concentration of between 2.5 and 3.5 mg/mL, such as 2.9 mg/mL, followed by administration for two weeks at a concentration of between 8 and 9 mg/mL, such as 8.6 mg/mL.
73. The compound for use, the method, the pharmaceutical composition or the use according to any one of the preceding items, wherein:
   a. between 0.025 and 0.075 mL/cm², such as 0.05 mL/cm², of the peptide or compound is administered to the individual affected by the skin condition or skin disorder during the first period of treatment, wherein the concentration of said peptide or compound is between 2.5 and 3.5 mg/mL, such as 2.9 mg/mL;
   b. between 0.05 and 0.15, mL/cm², such as 0.1 mL/cm², of the peptide or compound is administered to the individual affected by the skin condition or skin during the second period of treatment, wherein the concentration of said peptide or compound is between 2.5 and 3.5 mg/mL, such as 2.9 mg/mL;
   c. between 0.025 and 0.075 mL/cm², such as 0.05 mL/cm², of the peptide or compound is administered to the individual affected by the skin condition or skin during the third period of treatment, wherein the concentration of said peptide or compound is between 8 and 9 mg/ml, such as 8.6 mg/mL; and
   d. between 0.05 and 0.15, mL/cm², such as 0.1 mL/cm², of the peptide or compound is administered to the individual affected by the skin condition or skin during the fourth period of treatment, wherein the concentration of said peptide or compound is between 8 and 9 mg/mL, such as 8.6 mg/mL; optionally wherein each period of treatment is approximately one week,
   optionally wherein the compound or peptide is administered three times per week, such as every second or third day.
74. The compound for use, the method, the pharmaceutical composition or the use according to any one of the preceding items, wherein:
   a. between 0.1 and 0.2 mg/cm², such as 0.14 mg/cm², of the peptide or compound is administered to the individual affected by the skin condition or skin disorder during the first period of treatment;
   b. between 0.25 and 0.35 mg/cm², such as 0.29 mg/cm², of the peptide or compound is administered to the individual affected by the skin condition or skin during the second period of treatment;
   c. between 0.4 and 0.5 mg/cm², such as 0.43 mg/cm², of the peptide or compound is administered to the individual affected by the skin condition or skin during the third period of treatment; and
   d. between 0.8 and 0.9 mg/cm², such as 0.86 mg/cm², of the peptide or compound is administered to the individual affected by the skin condition or skin during the fourth period of treatment;
   optionally wherein each period of treatment is approximately one week,
   optionally wherein the compound or peptide is administered three times per week, such as every second or third day.
75. The compound for use, the method, the pharmaceutical composition or the use according to any one of the preceding items, wherein the concentration of the peptide or compound in said composition is between 0.05 wt % and 5 wt %, such as between 0.08 wt % and 1 wt %, such as between 0.8 wt % and 3 wt %, such as between 2.5 wt % and 9 wt %.
76. The compound for use, the method, the pharmaceutical composition or the use according to any one of the preceding items, wherein the concentration of the peptide or compound in said composition is between 0.05 wt % and 5 wt %, such as between 0.08 wt % and 1 wt %, such as between 0.08 wt % and 0.3 wt %, such as between 0.25 wt % and 0.9 wt %.
77. The compound for use, the method, the pharmaceutical composition or the use according to any one of the preceding items, wherein the concentration of the peptide or compound in said composition is 0.086 wt %, 0.29 wt % or 0.86 wt %.
78. The compound for use, the method, the pharmaceutical composition or the use according to any one of the preceding items, wherein the composition further comprises:
   a. EDTA, and
   b. an aqueous buffer,
      wherein
         i. the composition has a pH of at the most 8 and/or
         ii. the concentration of the compound in the composition is at least 0.08 wt %.
79. The compound for use, the method, the pharmaceutical composition or the use according to any one of the preceding items, wherein the composition further comprises:
   a. a polymer capable of forming a hydrogel when mixed with an aqueous solution, and
   b. an aqueous solution
      wherein
         i. the concentration of the compound in the composition is at least 0.08 wt % and/or
         ii. the polymer is present in said composition at a concentration of at least 0.05 wt %.

### EXAMPLES

### Introduction

In order to efficiently heal wounds, it is therefore not only critical to prevent and treat infections but also to address the unhealthy excessive part of the inflammation for which there is no commercially available treatment today. Local and systemic antibiotics and topical wound treatments based on dressings with anti-infective components, such as silver, do not address the excessive and harmful inflammation associated with wounds. Moreover, despite the widespread use of antiseptics for various wound indications such as EB, there is little clinical evidence demonstrating that such treatments improve wound healing or prevent infection. Furthermore, infection management is complicated by the decreasing effectiveness of antibiotics and antimicrobial agents due to antimicrobial resistance (AMR).

Initially found in wounds, thrombin-derived C-terminal peptides (TCP) of roughly 2 kDa, such as FYT21 (FYTHVFRLKKWIQKVIDQFGE) and HVF18 (HVFRLKKWIQKVIDQFGE), have been demonstrated to exert anti-endotoxic functions in vitro and in vivo (Saravanan et al., 2018; van der Plas et al., 2016; Kalle et al., 2012). TCP-25 (GKYGFYTHVFRLKKWIQKVIDQFGE), encompassing sequences of natural TCPs previously identified in human wounds has been shown to neutralize LPS in vitro and protect against Pseudomonas aeruginosa sepsis and LPS-mediated shock in experimental animal models, mainly via reduction of systemic cytokine responses (Saravanan et al., 2018; Kalle et al., 2012). A hydrogel containing TCP-25 treated both infection and inflammation in murine and porcine wound infection models with excellent results (Puthia et al., 2020). Bioactive cleavage fragments of the peptide were similar to those found in human wound fluid. The mode of action is well characterised and involves interactions in the μM range with LPS, other bacterial products, and the cell-receptor CD14, enabling a down-modulation of inflammation, while simultaneously exerting direct antimicrobial effects. Importantly, TCP-25 is acting on multiple multiresistant bacterial isolates in said models (Puthia et al., 2020).

The safety and tolerability of three dose levels of TCP-25 was tested in experimental wounds generated by the suction blister technique. Suction blister wounds are standardized with respect to wound depth (entire epidermal layer is ablated and dermis is exposed), size, and closure time. The mechanical nonbleeding excoriation elicits an innate immune response accompanied by increased blood flow and infiltration of the underlying dermis with inflammatory cells. Epidermal regeneration occurs from the edges and appendages (Ahlstrom et al., 2018). Interestingly, the inventors have found, that suction blister wounds may be useful as a model for studying EB, even though suction blister wounds lack of the genetic background in EB and also do not develop into non-healing wounds having a dysbiosis, with wounds typically colonised and chronically infected by bacteria such as Staphylococcus aureus, a major pathogen causing wound infections (Bar et al., 2021). However, the inventors have realised that suction blister wounds are similar to EB in other aspects, e.g. because they lack the epidermal component and have the same wound depth as types of EB. Therefore, the combination of application of TCP-25 on suction blister wounds captures relevant TCP-25 actions in wounds relevant for the EB indications.

### Example 1

We studied the effects of a hydrogel containing the immunomodulatory host defense peptide TCP-25 (GKYGFYTHVFRLKKWIQKVIDQFGE) on wound bacteria and inflammation in healthy test persons enrolled after informed consent. Blister wounds were formed on the medial aspect of the thigh, two blisters on each leg, defined as day 1. Each wound was treated with 0.15 mL of a sterile gel containing either TCP-25 (0.86, 2.9, or 8.6 mg/mL according to the dose group) or placebo (0 mg/mL TCP-25), applied topically at day 1. The composition of the gel is shown below.
Low strength: 0.86 mg/mL TCP-25, 1.35% Natrosol 250 HX, 2% Glycerol, in 10 mM Tris pH 7.
Middle strength: 2.9 mg/mL TCP-25, 1.2% Natrosol 250 HX, 2% Glycerol, in 10 mM Tris pH 7.
High strength: 8.6 mg/mL TCP-25, 1% Natrosol 250 HX, 2% Glycerol, in 10 mM Tris pH 7.

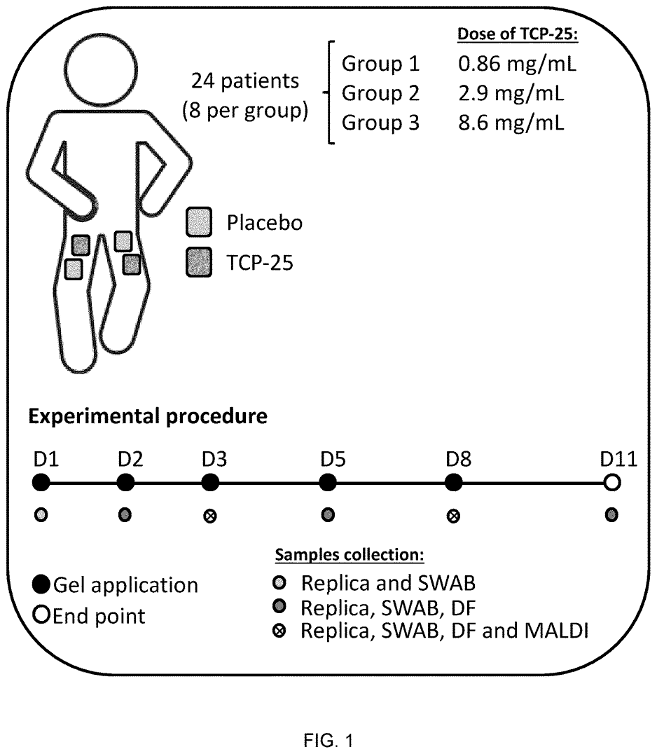

After application of the gel, each wound was dressed using a 2×2 cm Mepilex transfer (Mölnlycke health care) as primary dressing and covered with a secondary dressing (Tegaderm, 3M™). A secondary protective layer was applied, consisting of overlapping gauze swabs covered with a secondary Tegaderm dressing. Evaluations were performed days 2, 3, 5 and 8 where new gel was applied. Previous wound dressings was collected. Photos of the wound taken, sampling of the wound material via swabs and a sterile filter paper was done. New gel was applied, followed by dressing of each wound. At Day 11 photos of the wound area were taken, swabs and filter paper sampled, and previous wound dressing collected. At Day 11 the wounds were all healed (FIG. 1).

### Results

### Quantification of Bacteria

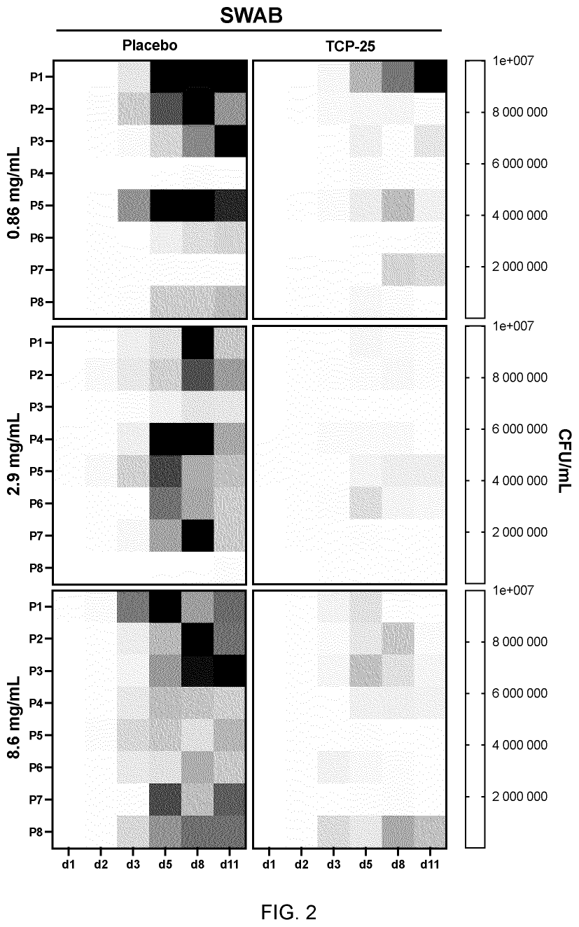

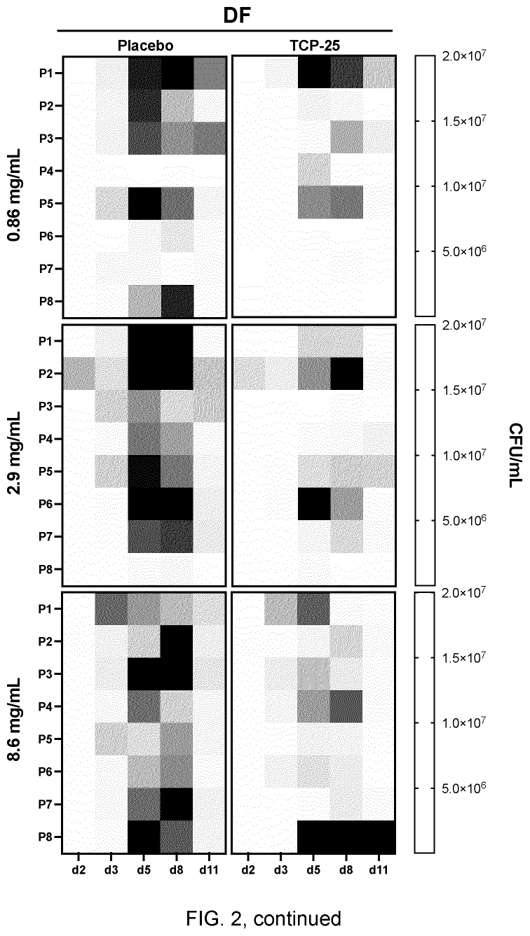

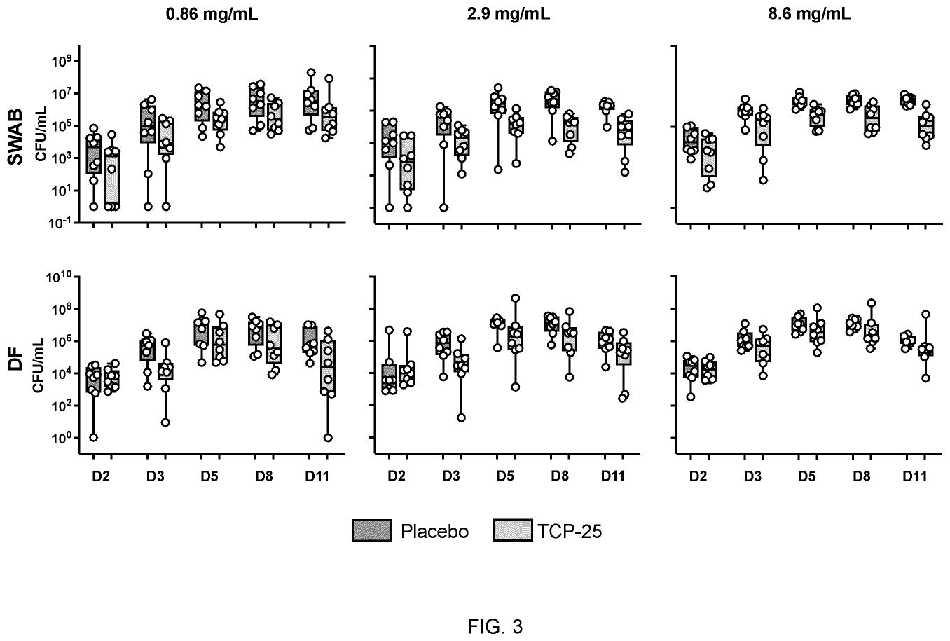

Bacteria were quantified from the wound surface and the dressing. The results showed that TCP-25 gel significantly reduced the increase in microbial burden during wound healing in both the dressing and at the wound surface (FIGS. 2 and 3). Notably, the bacterial reduction was observed at the three doses 0.86, 2.9 and 8.6 mg/ml. Inspection of the data showed that TCP-25 gel was able to control bacteria particularly at the wound surface (FIGS. 2 and 3).

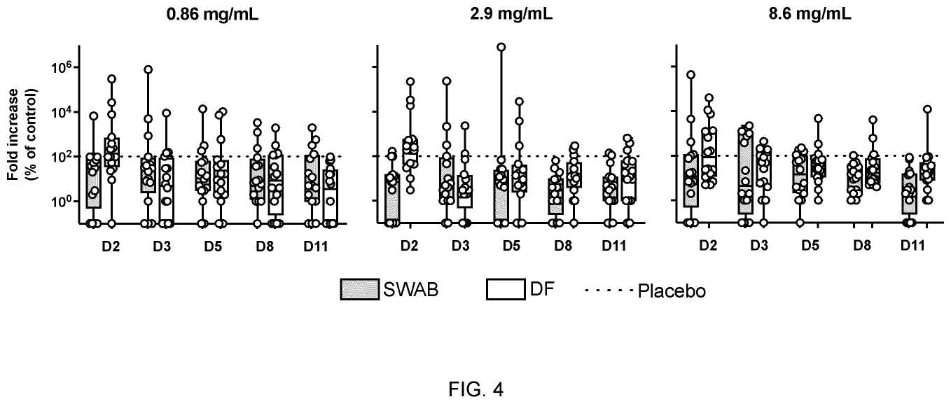

In order to analyse the relative reduction on each leg, the level of bacteria in the controls was normalized, and the bacterial level after TCP-25 treatment was expressed relative the control. The results showed that TCP-25 reduced the bacterial relative the adjacent control (FIG. 4).

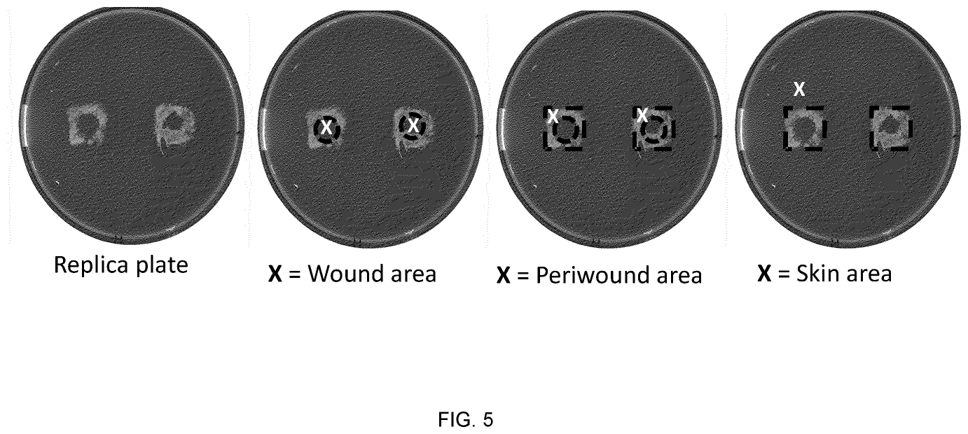

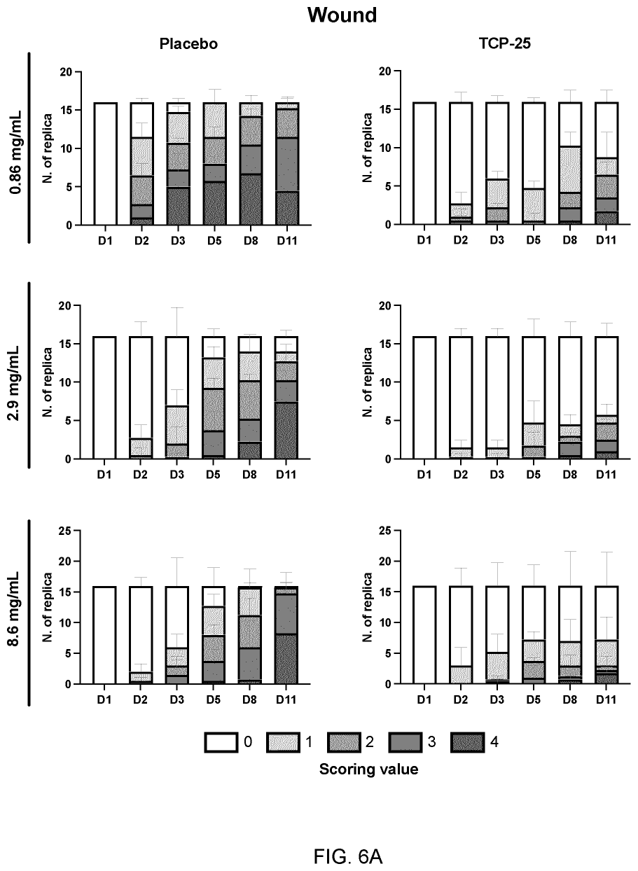

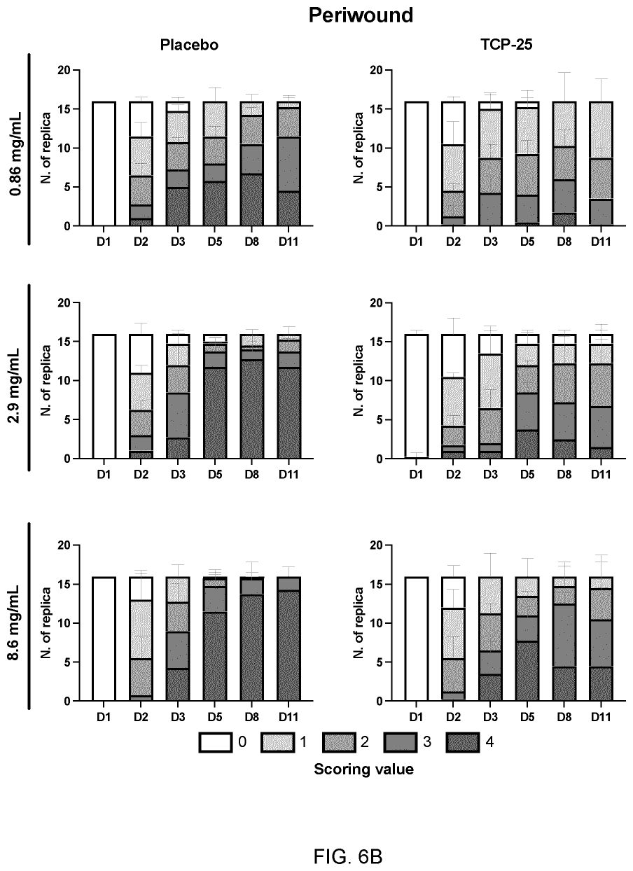

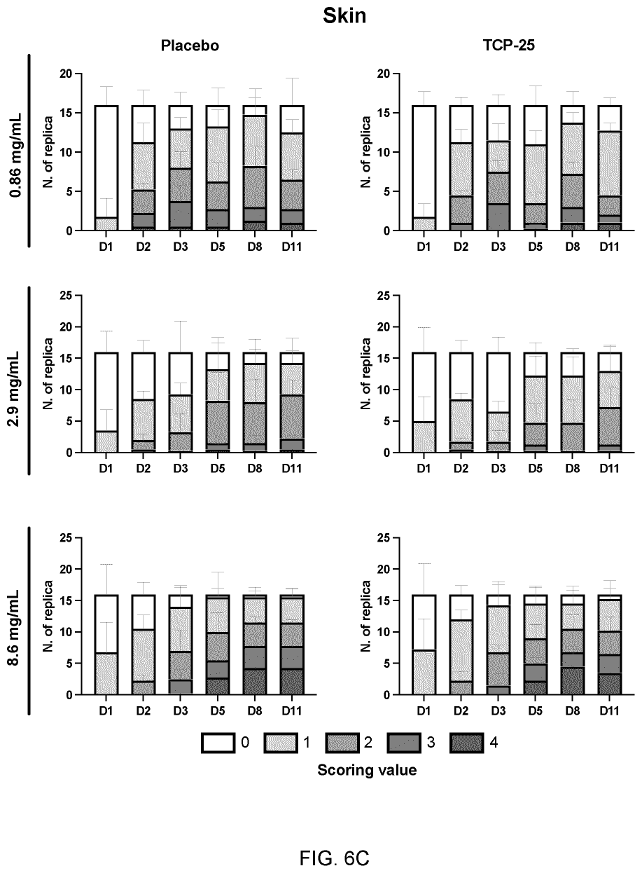

Analysis using a replica method (FIG. 5) provides spatial data of the bacteria in the wounds. FIG. 6A-C shows that TCP-25 gel reduced bacterial levels in the wound, and the periwound areas. The bacteria in the surrounding skin adjacent to the dressing remained unaffected.

### Identification of Bacterial Types

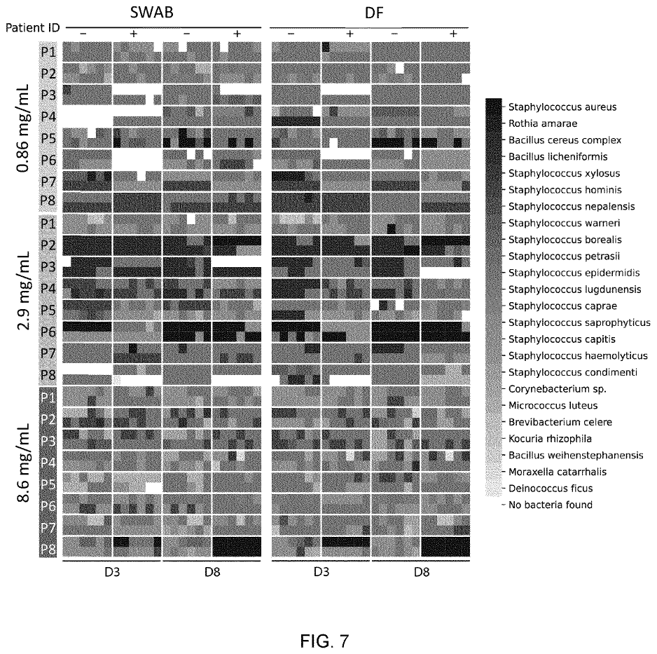

Bacteria was identified from the wound surface and the dressing at day 3 and 8. The results showed that there was a development of a microbiome with usually 1-3 types present. Commensal bacteria were dominating, and it was observed that there was a correspondence between the types found at the wound surface and in the dressing. Importantly, TCP-25 treatment did not change the overall composition of the commensal microbiome (FIG. 7).

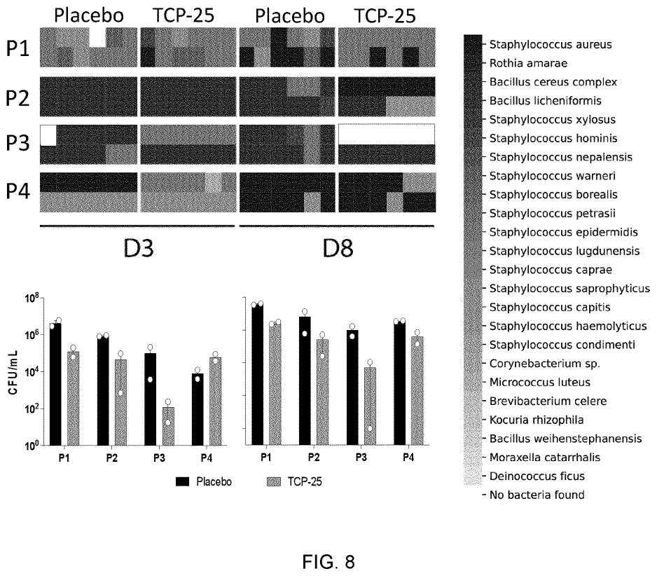

S. aureus can colonise and infect epidermal wounds. The bacterium was detected in several wounds. Importantly, TCP-25 treatment was able to reduce the bacterial levels particularly at the wound surface, hence demonstrating that the treatment controlled excessive levels of S. aureus (FIG. 8). A similar observation was made for wounds colonized with the species Bacillus (FIG. 8).

### Analysis of Wound Exudation

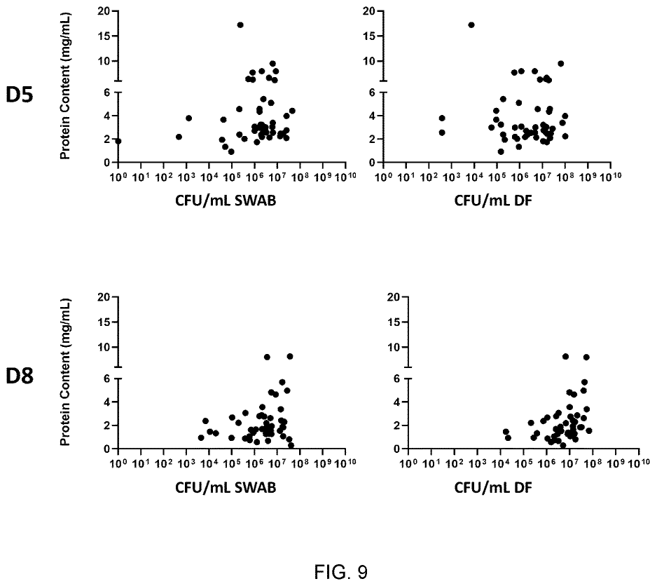

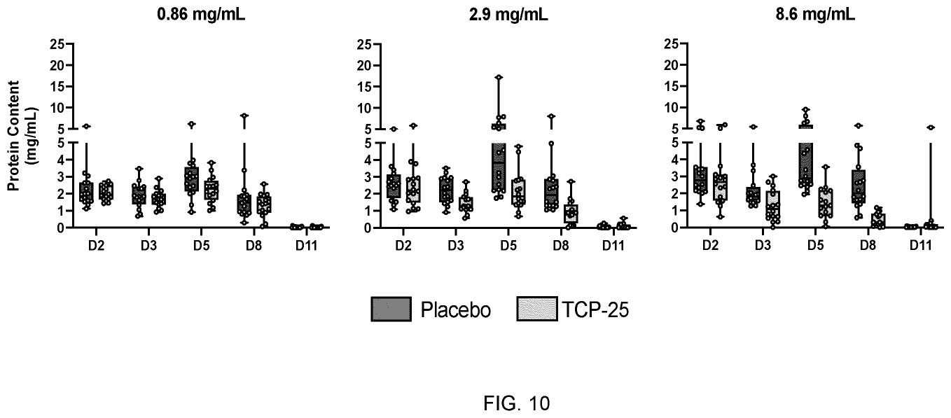

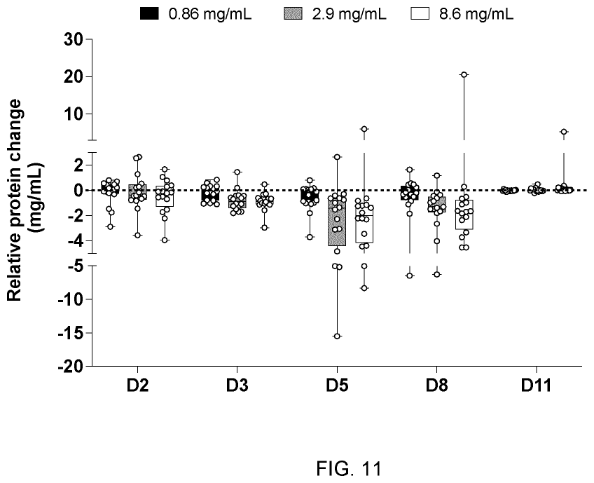

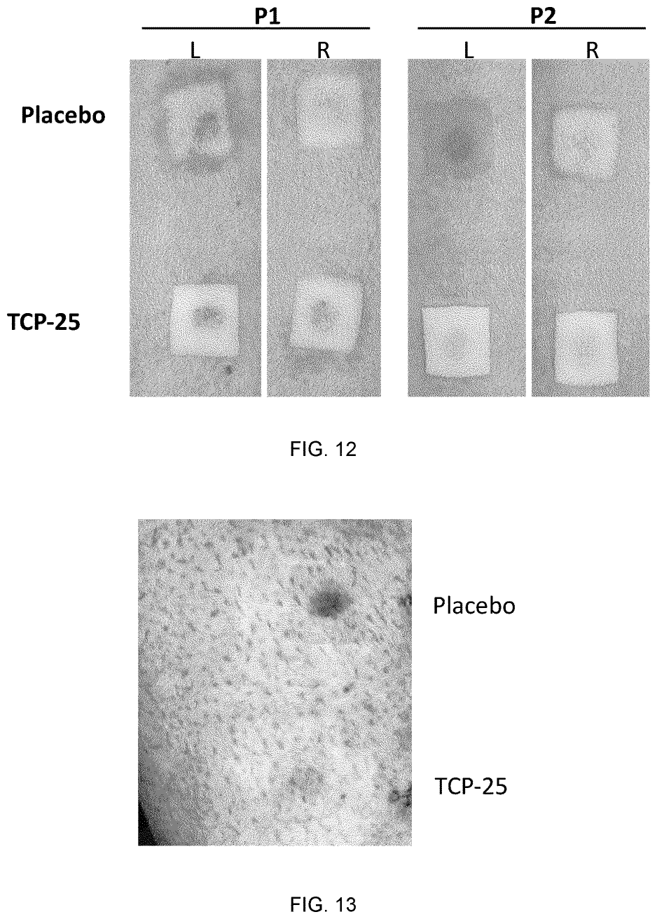

Wound exudation was determined by analysis of total protein content in the dressing fluid. The results showed that the commensal bacteria did not significantly influence the exudation during the study. Hence no correlation between bacterial levels and protein leakage was detected for the different days (FIG. 9). TCP-25 treatment reduced exudation particularly at day 5 and 8 in a dose dependent way (FIG. 10). The difference in the levels of exudation was calculated relative the adjacent control wound. 2.6 and 8.6 mg/ml TCP-25 reduced protein content, and notably, 8.6 mg/ml TCP-25 showed significant reductions, with only 20-30% exudation relative the control (FIG. 11). FIG. 12 illustrates the reduction by TCP-25 of leakage in the dressing.

### Analysis of Neutrophil Proteins in Wounds

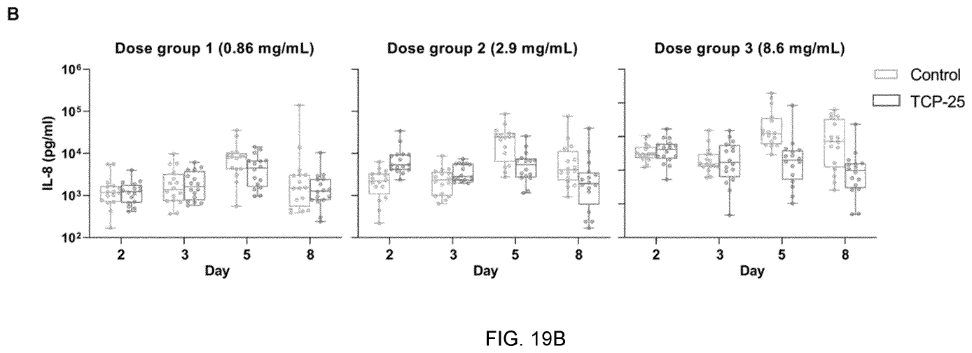

Next, we measured levels of several neutrophil-derived proteins by ELISA in dressing fluid samples. Generally, MPO levels were lower across most time points for all 3 doses of TCP-25 versus placebo, with the largest decreases occurring on Day 5 (FIG. 19A, upper panels). Similarly, all 3 doses of TCP-25 downregulated NE, primarily on Days 5 and 8, compared with the control (FIG. 19A, middle panels). Finally, TCP-25 decreased the levels of HBP at all time points at all doses, extinguishing HBP expression to near-zero levels (FIG. 19A, lower panels).

Notably, although not a neutrophil protein per se early in the inflammatory phase of wound healing, the content of the chemotaxis-inducing IL-8 in the wounds declined with all 3 doses of TCP-25 by Days 5 and 8 (FIG. 19B).

HBP is a major factor known for causing vascular leakage, and is also consistently elevated in chronic non-healing wounds, characterized by their persistent inflammation and wound leakage. The dose-dependent significant reduction in HBP levels could therefore underlie the observed reduction of wound exudation in this study.

Significantly reduced leakage has also been observed in patients suffering from venous leg ulcers. When treated with a TCP-25 formulation as described here in Example 1, a significant reduction in leakage from the treated venous leg ulcers has been observed.

### Analysis of Scarring

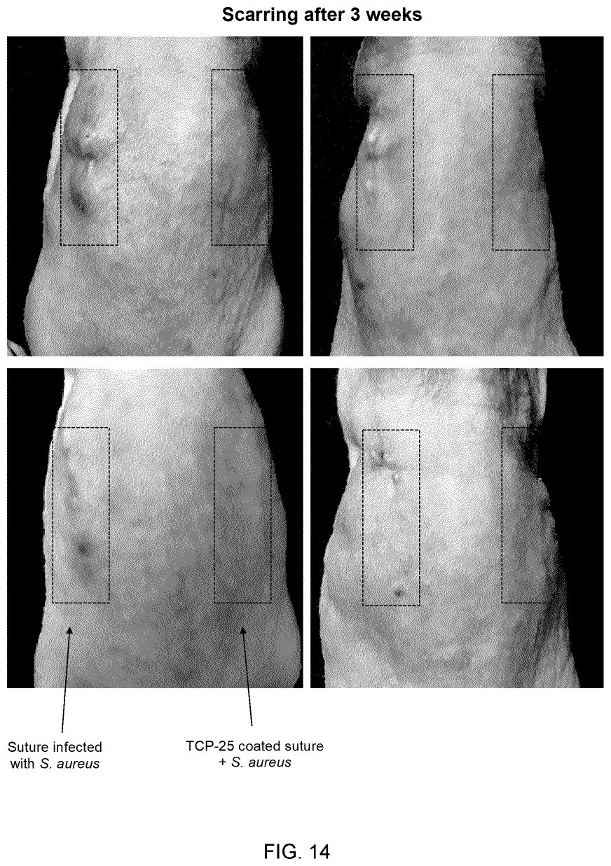

In a separate experiment performed as above, scarring and pigmentation was analysed. The results showed that TCP-25 reduced pigmentation at day 11 (FIG. 13). To further study TCP-25 effects on scarring, a mouse model was employed. A suture was contaminated with S. aureus, implanted subcutaneously, and then subjected buffer or TCP-25. TCP-25 significantly reduced scarring (FIG. 14).

### Conclusions

The results showed that TCP-25 reduced excessive bacteria already at the lowest dose, while the spectrum of microbes (i.e. natural skin microbiome) was maintained. It also controlled excessive S. aureus levels. Further, we show that TCP-25 reduces wound leakage in a dose-dependent manner. TCP-25 however reduced bacteria to a similar extent at all three doses. The leakage reduction was therefore not seen at the lowest dose that showed bacterial reduction, hence demonstrating that TCP-25's effect on exudation and its effect on bacterial reduction are two separate aspects of its action.

EB patients suffer from both microbiome dysbiosis and wound leakage, and importantly, the wound leakage and associated maceration of the skin can lead to blisters increasing in size and increase infection risk. It can be concluded that TCP-25 can be used to regulate skin microbiome dysbiosis and wound leakage to improve wound healing and decrease blister/wound size and infection.

The results also showed that TCP-25 can reduce pigmentation and scarring.

### Methods

### Suction Blister Wound Formation

Blister wounds were formed on the medial aspect of the thigh, two blisters on each leg with a 6 cm distance in between them. Before wounding, hair at the wound sites was shaved. Each wound site was wiped with an ethanol-soaked gauze. The exact wound sites were marked using a skin marker.

The blisters were made using the Model NP-4 (Electronic Diversities, Finksburg, MD) suctioning device (11) operating in the negative pressure range 8-16 inchHg with heating for optimal blister formation. Blister wound diameter was 10 mm. The device was run until all blisters have formed, which takes approximately 60-70 minutes. The roof of the blister was excised using a sterile forceps and scissors.

### Interventions

Treatment: Each wound received 0.15 mL of a sterile gel containing either TCP-25 (0.86, 2.9, or 8.6 mg/mL according to the dose group) or placebo (0 mg/mL TCP-25), applied topically.

Dressing: After application of the gel, each wound was dressed using a 2×2 cm Mepilex transfer (Mölnlycke health care) as primary dressing and covered with a secondary dressing (Tegaderm, 3M™). A secondary protective layer was applied, consisting of overlapping gauze swabs covered with a secondary Tegaderm dressing.

### Allocation

Each subject received four suction blister wounds, two on each thigh. Wounds were labelled R1 (right thigh, distal), R2 (right thigh, proximal), L1 (left thigh, distal) and L2 (left thigh, proximal). (FIG. 1)

A computer-generated randomization list was created using SAS Proc Plan, SAS Version 9.4. The randomization list contained subject number, wound position, thigh (left/right) and treatment and was kept by the randomizer in a sealed envelope until database lock. On Day 1 the subjects in each dose group (n=8) was randomised with regards to the treatment allocation of each wound. Two wounds per patient received TCP-25, and the other two received placebo. The proximal wound (R1 or L1) of one thigh and the distal wound of the other thigh (L2 or R2) received the same treatment, according to the assigned treatment allocation. Two syringes per patient were prepared by an unblinded nurse in a nearby medical room, one with TCP-25 and one with placebo, labelled with the randomisation number of the patient and the identification of two wounds (R1+L2 or R2+L1) according to the assigned treatment allocation.

### Blinding

This was a double-blind study and the allocation of treatments was not disclosed until clean file declared and the database locked.

### Bacterial Quantification

### Swab Procedure

The swab was pre-wetted with phosphate-buffered saline. Using a twisting motion the swab was rotated 10 times covering the whole wound while using only light pressure, and then placed into a microtube with 0.5 mL of sterile phosphate buffered saline (PBS) and kept on ice. The tube were vortexed to dislodge bacteria from the swab. The amount of bacteria was quantified as described below.

### Dressing Extraction Procedure

The Mepilex dressing was removed from the wound and then placed into an empty conical tube and kept on ice. Each tube containing the dressing was dried and weighed, and the dressing was then be removed and placed into a 5 ml syringe fixed with a stopper. Cold sterile Tris buffer (2 mL) was added to the syringe and the syringe will be vortexed for 5 minutes. The fluid was then extracted from the dressing by pressing the plunger down to squeeze the fluid out into the original dressing collection tube. The extracted fluid was kept on ice. The amount of bacteria, protein content neutrophil proteins and cytokines was quantified as described below.

### Quantitative Bacterial Counts

The swab and dressing fluid, collected as described above, was diluted with sterile PBS to make seven 10-fold serial dilutions (10×, 100×, 1000×, 10000×, 100000×, 1000000×, 10000000×). From the undiluted sample and from each of the dilutions, six 10 μL drops were plated on an agar plate. The plates were incubated at 37° C. in 5% CO₂ overnight. The next morning, the number of colonies was counted and recorded.

### Replica Procedure

A sterile (autoclaved) 12.5 cm filter paper (Whatman) was pre-wet with 3 ml of sterile PBS before being transferred to the wound. The filter paper was positioned to cover both wounds on the leg and, using the lid of the petri dish, was gently pressed on the wounds for 1 minute with a rocking motion to ensure equal contact of the wounds with the filter paper. The filter paper was then placed face-down on a 15 cm agar plate and incubated at room temperature for at least 1 hour. The filter paper was then discarded from the plate and the plate was incubated at 37° C. in 5% CO₂ overnight.

### Imaging and Image Processing

The next day the filter paper was imaged using ChemiDoc MP Imaging System (BioRad) using the Stain Free Blot imaging method and a 0.386 s exposure time, yielding gray-scale images of all replicas. The images were then trimmed to remove excess background. Using ImageJ Fiji, an individual threshold value for each gray-scale replica image was chosen to acquire a binary image. The threshold was manually chosen in such a way that the bright edges of each replica agar plate would all be of comparable apparent intensity and nothing except for microbial colonies is lighting up in the wound area.

### Manual Scoring

Four independent people graded the number of microbial colonies of all control wounds on a 5-point scale ranging from 0-4, with 0 being the lowest amount of bacteria and 4 being the highest amount of bacteria. The assessors assigned a separate grade to the central area where the wound was located, the dressing area, and the surrounding area.

### Identification of Bacteria

Swab and dressing fluid, extracted as described above, were streaked on a blood agar plate and incubated at 37° C. overnight. Bacteria samples were analyzed with MALDI-TOF MS on a M@ldi LR (Waters/Micromass, Milford, MA, USA) in linear mode. For the MALDI-TOF MS analysis, a spectrum of 100 summed laser shots was acquired for each sample spot. MassLynx 4.1 was used for controlling the Waters instrument. The bacterial identification experiments were performed on an ultraflextreme MALDI-TOF/TOF (Bruker Daltronik GmbH, Germany) in linear mode in a mass range of 2 to 20 kDa and were analyzed using FlexControl and MALDI Biotyper 3.1 software (Bruker Daltronik GmbH, Germany). The identification results were evaluated according to the manufacturer's instructions. Scores values above 2 suggested a probable identification to the species level; scores between 1.7 and 2 indicated genus identification, and scores of 1.7 and below were considered to be unreliable.

### Analysis of Wound Exudates

Wound fluid was analysed for protein content using the bicinchoninic acid (BCA) assay (Thermo Fisher) according to the manufacturer's directions using bovine serum albumin as the standard.

### Quantification of Neutrophil Proteins

Human neutrophil elastase (NE) and human myeloperoxidase (MPO) were measured using ELISA kits from R&D Systems (Minneapolis, MN, USA); heparin-binding protein (HBP) was determined using an ELISA kit from Aviva Systems Biology (San Diego, CA, USA). Samples were according to the manufacturer's instructions in duplicate. ELISA signals were read at 450 nm on a microplate reader (BioRad). A standard curve was established with reference standards in GraphPad Prism 9 to calculate the concentration in each sample.

IL-8 was measured using a V-PLEX Proinflammatory Panel 1 (human) kit (Meso Scale Diagnostics, art. no: K15049D) per the manufacturer's instructions at a 5× dilution. Samples for which IL-8 was above the upper limit of detection were analyzed again using the U-PLEX Biomarker Group 1 (human) kit (Meso Scale Diagnostics, art. no: K151TYK-2) at a higher dilution. The concentration of IL-8 was calculated using the MSD discovery Workbench analysis software. Samples with a concentration below the detection limit were not quantified by the analysis software and were assigned a value of zero for all analyses.

For all measurements of neutrophil proteins and IL-8, the assessor was not blinded to the identity of the assigned intervention.

### Wound Imaging

A centimeter scale ruler was stuck on to the skin near the wounds. A Canfield camera was used with the close-up scale, held so that both wounds on the same leg were visible. Two images were taken of each set of wounds on each leg.

### Example 2

### Methods

### Mouse Model of EB

Two mouse models of EB will be used for this study.

### 1. B6.129X1-Lamc2Jeb/DcrJ Stock No. 025467

This Lamc2^{jeb} mutant mouse strain is useful in studies of EB and is a model for Non-Herlitz Type Junctional Epidermolysis Bullosa. The Lamc2 gene encodes for the gamma-2 subunit of laminin 5, which is an extracellular matrix glycoprotein found in cutaneous basement membranes. These mice carry a spontaneous mutation of Lamc2 that arose on the 129×1 background and is a single retroviral insertion (murine leukemia virus long terminal repeat) in intron 18.

### 2. C57BL/6J-Col17a1em8Dcr/J Stock No. 033908

These TALEN-derived mice carry a 10 bp deletion in exon 50 of the Col17a1 (collagen, type XVII, alpha 1). Col17a1 (collagen, type XVII, alpha 1) encodes the alpha chain of type XVII collagen. Collagen XVII is a transmembrane protein involved in the adhesion of keratinocytes to the basement membrane. Mutations in Col17a1 are associated with human junctional epidermolysis bullosa (JEB), a genetic condition that causes skin fragility and blistering. This TALEN-derived allele has a 10 bp deletion in exon 50.

### Experimental Approach and Treatment

Group 1 (No infection)
1. TCP-25 gel will be applied once daily on a defined area of the tail and ears.
2. The area will be covered with a secondary dressing and a final adhesive dressing will be applied.
3. In control animals, gel without TCP-25 will be applied.
4. Treatment will end either on day 3, day 7, or day 14.

### Group 2 (Addition of Infection)

1. A solution containing S. aureus or P. aeruginosa will be applied to the defined area and treatment will be started 30 minutes later.
2. TCP-25 gel will be applied once daily.
3. The area will be covered with a secondary dressing and a final adhesive dressing will be applied.
4. In control animals, gel without TCP-25 will be applied.
5. Treatment will end either on day 2, day 7, or day 14.

### Group 3 (Recurrence)

The purpose of this group is to study if treatment has any long-lasting benefit on skin microbiome or histology.
1. Like group 1, mice will be treated for 7 days.
2. After 7 days of no treatment, at 14 days, all readouts will be made to evaluate efficacy.

### Readouts:

1. Clinical observation (at each dressing change).
2. Photography (at each dressing change).
3. Swab for CFU (Days 1, 2, 4, 7, 10, 14).
4. Swab for microbiome analysis (Days 7 and 14).
5. Histology (day 2, day 7, or day 14). Scoring will be done.
6. Immunohistochemistry (day 2, day 7, or day 14).
7. IVIS imaging in group 2 (at dressing change).
8. Tissue collection (at termination) for DNA or Transcriptomics.

### Expected Results

### Microbiome Control

As in the study on human epidermal wounds, TCP-25 achieves a significant reduction of excessive levels of bacteria. In non-infected wounds, TCP-25 controls excessive colonization with pathogenic S. aureus. In the infection models with S. aureus and P. aeruginosa, TCP-25 is able to reduce bacterial levels and blister formation.

### Microbiome Identities

Analysis using Maldi TOF and also 16s DNA analysis shows that TCP-25 prevents excessive dysbiosis.

### Barrier Damage

Since exudation is difficult to assess in the mouse model, histological evaluation was performed. The results showed that TCP-25 normalised the skin barrier.

### Example 3—Epidermolysis Bullosa Mouse Model

The mouse model B6. 129×1-Lamcjeb/DcrJ of EB was studied based on the description in Example 2. However, additional studies were performed and modifications were made as outlined in detail in this example.

### Material and Methods

### Epidermolysis Bullosa Mouse Model

B6.129X1-Lamc2jeb/DcrJ (Strain #: 025467). This Lamc2^{jeb} mutant mouse strain is useful in studies of epidermolysis bullosa and is a model for Non-Herlitz Type Junctional Epidermolysis Bullosa (Bubier et al., 2010; Sproule et al., 2014; JAX stock #025467). In homozygous mice, skin blistering occurs approximately around 13 weeks. Mice were housed in standard conditions in disposable IVC Caging system (Inovive).

### Epidermolysis Bullosa Mouse Model—No Induced Wounding or Infection

Mouse model (Lamc2jeb mice) of human EB was used (15-16 weeks old). Procedures were performed under isoflurane anaesthesia (4% induction, 2% maintenance). Using a trimmer, hair from the mouse dorsum were trimmed and skin was cleaned with sterile gauze. Left side of the mice was treated with 200 μL TCP-25 gel (8.6 mg/mL) and right side was treated with control gel. A 1.5×1.8 cm polyurethane primary dressing (Mepilex Transfer) was applied on each side on the skin which was followed by a secondary dressing (Tegaderm). Finally, an adhesive dressing (Vet tape) was applied to secure dressings underneath. Mice were treated on days 0, 2, 5, 8 and 11. Swab samples and dressings were collected at each dressing change and were analyzed for CFU and cytokines. For CFU analysis, 500 μL of PBS was added to either swab or dressing samples and serial dilutions were plated on THA (Todd-Hewitt Agar) plates and incubated overnight at 37° C. Wound fluid extractions from dressings were analysed for cytokines using V-PLEX Cytokine Panel 1 Mouse Kit (MSD) following manufacturers protocol. One day after the last treatment, experiment was terminated, and skin biopsies were collected. Biopsies were fixed overnight in 10% neutral-buffered formalin. Using standard procedures, fixed tissues were paraffin embedded, sectioned, and stained for H&E.

### Epidermolysis Bullosa Mouse Model—Punch Wound

Mouse model (Lamc2jeb mice) of human EB was used (15-16 weeks old). Procedures were performed under isoflurane anaesthesia (4% induction, 2% maintenance). Using a trimmer, hair from the mouse dorsum were trimmed and skin was cleaned with sterile gauze. Using 4 mm biopsy punch, two full-thickness wounds were made on the left and right side (4 mm diameter). Left wound was treated with 200 μL TCP-25 gel (2.9 or 8.6 mg/mL) and right side was treated with control gel. A 1.5×1.8 cm polyurethane primary dressing (Mepilex Transfer) was applied on each wound which was followed by a secondary dressing (Tegaderm). Finally, an adhesive dressing (Vet tape) was applied to secure dressings underneath. Mice were treated on days 0, 1, 2, 4, 6 and 8. Swab samples and dressings were collected at each dressing change and were analyzed for CFU and cytokines. For CFU analysis, 500 μL of PBS was added to either swab or dressing samples and serial dilutions were plated on THA (Todd-Hewitt Agar) plates and incubated overnight at 37° C. Wound fluid extractions from dressings were analysed for cytokines using V-PLEX Cytokine Panel 1 Mouse Kit (MSD) following manufacturers protocol. One day after the last treatment, experiment was terminated, and skin biopsies were collected. Biopsies were fixed overnight in 10% neutral-buffered formalin. Using standard procedures, fixed tissues were paraffin embedded, sectioned, and stained for H&E.

Epidermolysis Bullosa Mouse Model—Punch Wound Infected with Staphylococcus aureus

Mouse model (Lamc2jeb mice) of human EB was used (15-16 weeks old). Procedures were performed under isoflurane anaesthesia (4% induction, 2% maintenance). Using a trimmer, hair from the mouse dorsum were trimmed and skin was cleaned with sterile gauze. Using 4 mm biopsy punch, two full-thickness wounds were made on the left and right side (4 mm diameter). Wounds were infected with a bioluminescent S. aureus (SAP229). Left wound was treated with 200 μL TCP-25 gel (8.6 mg/mL) and right side was treated with control gel. A 1.5×1.8 cm polyurethane primary dressing (Mepilex Transfer) was applied on each wound which was followed by a secondary dressing (Tegaderm). Finally, an adhesive dressing (Vet tape) was applied to secure dressings underneath. Mice were treated on days 0, 1, 2, 3, and 6. Swab samples and dressings were collected at each dressing change and were analyzed for CFU and cytokines. At indicated days, during dressing change, in vivo bioimaging of infection was performed using IVIS imaging system under luminescent imaging mode. Bioluminescent signals from the mice were acquired and quantified using Living Image 4.0 Software (PerkinElmer). For CFU analysis, 500 μL of PBS was added to either swab or dressing samples and serial dilutions were plated on THA (Todd-Hewitt Agar) plates and incubated overnight at 37° C. Wound fluid extractions from dressings were analysed for cytokines using V-PLEX Cytokine Panel 1 Mouse Kit (MSD) following manufacturers protocol. One day after the last treatment, experiment was terminated, and skin biopsies were collected. Biopsies were fixed overnight in 10% neutral-buffered formalin. Using standard procedures, fixed tissues were paraffin embedded, sectioned, and stained for H&E.

### Preparation of TCP-25 Gel and Control Gel

Control gel: Natrosol™ 250HX (Ashland Inc., UK) (1.6% w/v) was dissolved in 1.21 g/L Tris-HCl at pH 7 containing 25 g/L glycerol and then sterilized by autoclaving (Phase A).

2.9 mg/mL gel: Natrosol™ 250HX (Ashland Inc., UK) (1.6% w/v) was dissolved in 1.21 g/L Tris-HCl at pH 7 containing 25 g/L glycerol and then sterilized by autoclaving (Phase A). TCP-25 was dissolved in water containing 25 g/L glycerol at final concentration of 20.0 mg/mL. The solution of TCP-25 was filtered with 0.2 μm filter (Pall KA02ECVP8G Mini Kleenpak Filter Capsule, Life Sciences, NY, USA) and then corrected for Tris content and pH. The final composition of TCP-25 solution (named Phase B) contained 11.4 g/L TCP-25, 25 g/L glycerol in 1.21 g/L Tris-HCl at pH 7. Phase B was mixed with Phase A in a ration 1:3. The final gel contained 2.9 mg/ml TCP-25, 1.37% Natrosol™ 250HX, 1.21 g/L Tris-HCl, 25 g/L glycerol at pH 7.

8.6 mg/mL gel: Natrosol™ 250HX (Ashland Inc., UK) (1.33% w/v) was dissolved in 1.21 g/L Tris-HCl at pH 7 containing 25 g/L glycerol and then sterilized by autoclaving (Phase A). TCP-25 was dissolved in water containing 25 g/L glycerol at final concentration of 60.0 mg/mL. The solution of TCP-25 was filtered with 0.2 μm filter (Pall KA02ECVP8G Mini Kleenpak Filter Capsule, Life Sciences, NY, USA) and then corrected for Tris content and pH. The final composition of TCP-25 solution (named Phase B) contained 34.4 g/L TCP-25, 25 g/L glycerol in 1.21 g/L Tris-HCl at pH 7. Phase B was mixed with Phase A in a ration 1:3. The final gel contained 8.6 mg/mL TCP-25, 1.37% Natrosol™ 250HX, 1.21 g/L Tris-HCl, 25 g/L glycerol at pH 7.

### Primary and Secondary Dressing

A 1.5×1.8 cm polyurethane primary dressing (Mepilex Transfer) was applied on each wound which was followed by a secondary dressing (Tegaderm). Finally, an adhesive dressing (Vet tape) was applied to secure dressings underneath.

### CFU and Cytokines Analysis of Swab and Dressing

For CFU analysis, 500 μL of PBS was added to either swab or dressing samples and serial dilutions were plated on THA (Todd-Hewitt Agar) plates and incubated overnight at 37° C. Wound fluid extractions from dressings were analysed for cytokines using V-PLEX Cytokine Panel 1 Mouse Kit (MSD) following manufacturers protocol.

### Skin Biopsy

Wound area including surrounding skin was harvested using a scissors. Harvested skin was placed on absorbent paper to prevent curling, which were fixed overnight in 10% neutral buffered formalin.

### Hematoxylin and Eosin (H&E) Staining and Microscopy

After serial dehydration, the tissue was embedded in paraffin blocks, sectioned at 5 μm, and stained with hematoxylin and eosin (H&E). Samples were imaged with bright field microscopy (Axioplan2, Zeiss, Germany) under 100× and 200× magnifications.

### Histology Score

For mouse tissue, four random microscopic views (100×) from each H&E-stained skin tissue section were scored. The histological scoring was based on epithelization, inflammatory cells, abscesses and tissue architecture. Scoring was based on a scale of 0-5. For each wound tissue section, 4-5 areas were examined under 100× magnification.

### Punch Biopsy

Procedures were performed under isoflurane anaesthesia (4% induction, 2% maintenance). Using a trimmer, hair from the mouse dorsum were trimmed and skin was cleaned with sterile gauze. Using 4 mm biopsy punch, two full-thickness wounds were made on the left and right side (4 mm diameter).

### In Vivo Bioimaging

Procedures were performed under isoflurane anaesthesia (4% induction, 2% maintenance). At indicated days, during dressing change, in vivo bioimaging of infection was performed using IVIS imaging system under luminescent imaging mode. Bioluminescent signals from the mice were acquired and quantified using Living Image 4.0 Software (PerkinElmer).

### Wound Healing Rate

Wound healing rate (WHR) was calculated following the equation: [(Ai−Af)/Ai], where Ai represents the initial wound area and Af represents the final area/measurement (Reference: Masson-Meyers D S, Andrade T A M, Caetano G F, Guimaraes F R, Leite M N, Leite S N, Frade M A C. Experimental models and methods for cutaneous wound healing assessment. Int J Exp Pathol. 2020 February; 101 (1-2): 21-37. doi: 10.1111/iep.12346. Epub 2020 Mar. 30. PMID: 32227524; PMCID: PMC7306904). Wound area was measured using Image J software.

### Results

### Epidermolysis Bullosa Mouse Model—No Induced Wounding or Infection

The mouse model (Lamc2jeb mice) of human EB as described in the materials and methods above was used for the experiment. Briefly, hair from the mouse dorsum were trimmed and skin was cleaned with sterile gauze. Left side of the mice was treated with TCP-25 gel (8.6 mg/mL) and right side was treated with control gel. Swab samples and dressings were collected at each dressing change. After ten days of treatment regimen, skin biopsies were collected.

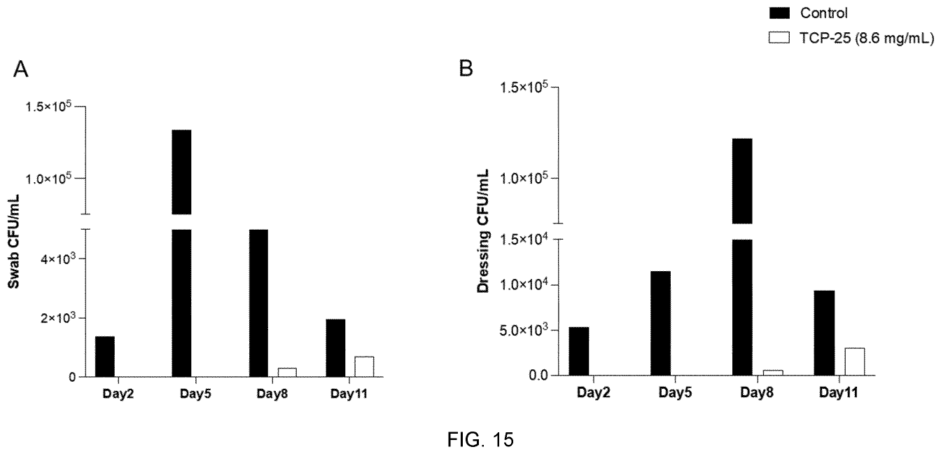

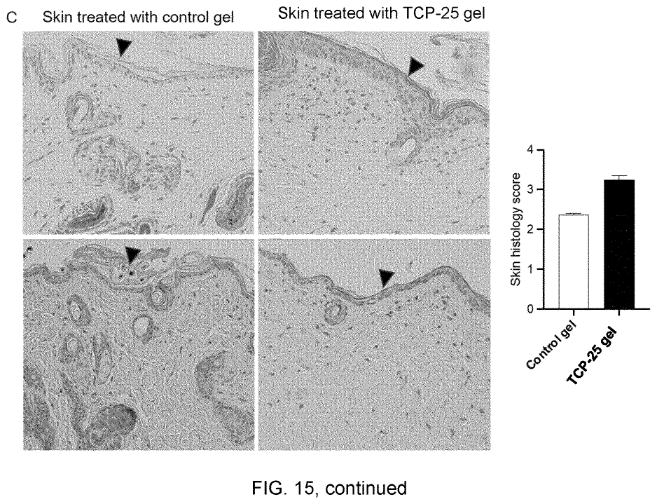

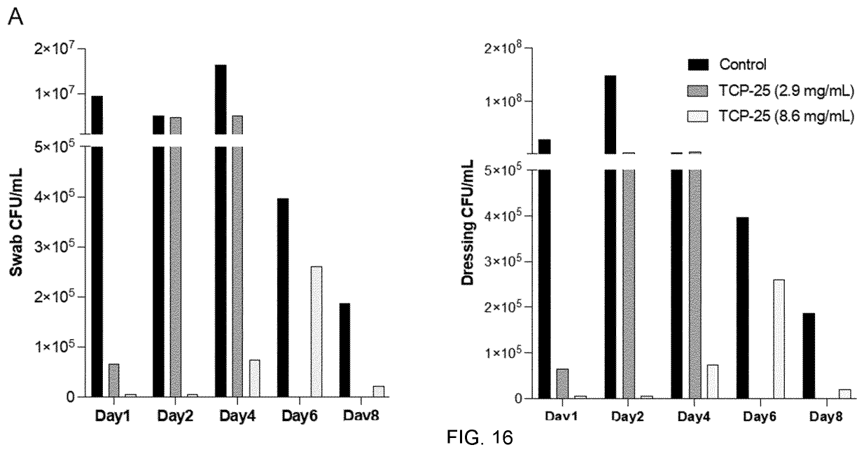

A reduction in CFUs was noticed in swab and dressing samples from TCP-25 gel treated mice as compared to mice treated with the control gel (FIG. 15A and FIG. 15B). As can be seen in the figure, no CFUs were detectable in the swab and dressing samples at Day 2 and 5 for mice treated with TCP-25 gel.

Representative H&E stained photographs of epidermis and dermis of TCP-25 gel treated and control gel treated mice revealed an immature epidermis and dermis with poor architecture in the control gel treated area, while the TCP-25 gel treated skin showed comparatively mature epidermis with improved dermal architecture (FIG. 15C). Furthermore, an improved skin histology score was observed in the skin of TCP-25 gel treated mice as compared to control mice (FIG. 15D).

### Epidermolysis Bullosa Mouse Model—Punch Wound

The mouse model (Lamc2jeb mice) of human EB as described in the materials and methods above was used for the experiment. Briefly, hair from the mouse dorsum were trimmed and skin was cleaned with sterile gauze. Under anaesthesia, two punch biopsy full-thickness wounds were made on the left and right side. Left wound of the mice was treated with TCP-25 gel (2.9 or 8.6 mg/mL) and right wound was treated with control gel. After 7 days of treatment regimen, wound biopsies were collected. Wound swab samples and dressings were collected at each dressing change.

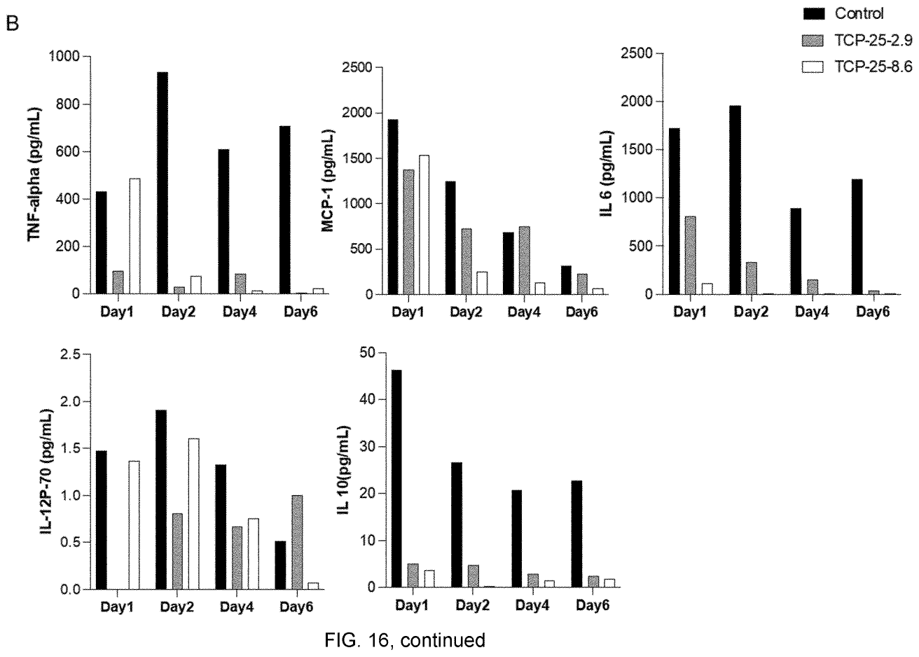

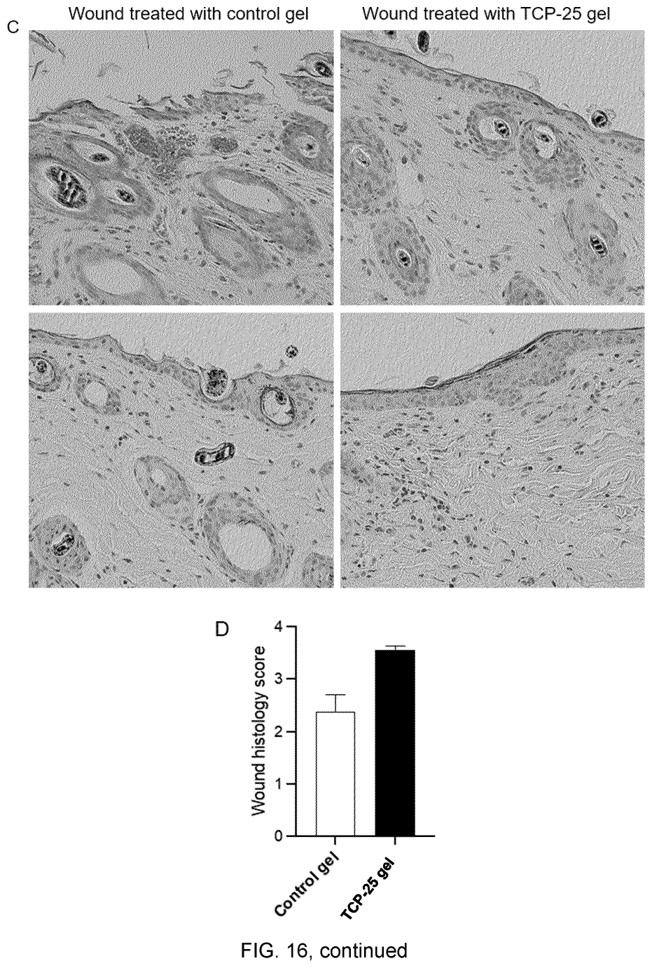

A reduction in CFUs was noticed in swab and dressing samples of TCP-25 gel treated mice as compared to mice treated with the control gel (FIG. 16A). Additionally, a reduction in wound fluid proinflammatory cytokines was noticed in TCP-25 gel treated mice as compared to mice treated with the control gel (FIG. 16B).

Representative H&E stained photographs of epidermis and dermis of TCP-25 gel treated and control gel treated mice revealed an immature epidermis and dermis with poor architecture in the control gel treated area, while the TCP-25 gel treated skin showed comparatively mature epidermis with improved dermal architecture (FIG. 16C). Furthermore, an improved wound histology score was observed in TCP-25 gel treated mice as compared to control mice (FIG. 16D).

Epidermolysis Bullosa Mouse Model—Punch Wound Infected with Staphylococcus aureus

The mouse model (Lamc2jeb mice) of human EB as described in the materials and methods above was used for the experiment. Briefly, hair from the mouse dorsum were trimmed skin was cleaned with sterile gauze. Under anaesthesia, two punch biopsy full-thickness wounds were made on the left and right side. Wound were infected with a bioluminescent S. aureus (SAP229). Left wound of the mice was treated with TCP-25 gel (8.6 mg/mL) and right wound was treated with control gel. In vivo bioimaging of infection was performed using IVIS imaging system. After 5 days of treatment regimen, wound biopsies were collected. Wound swab samples and dressings were collected at each dressing change.

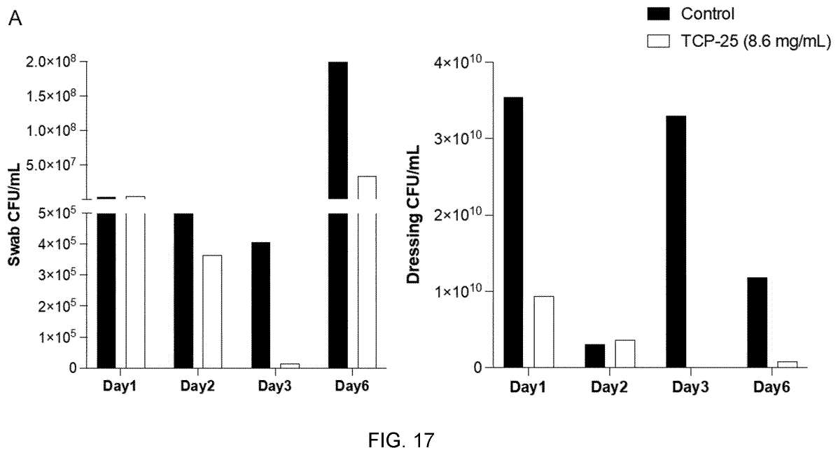

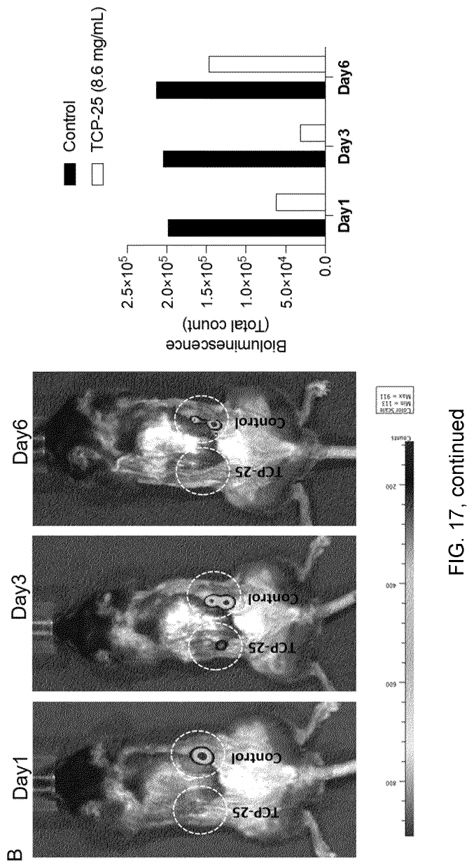

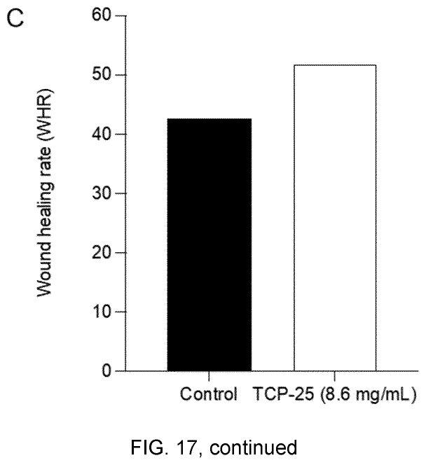

A reduction in CFUs was noticed in swab and dressing samples of TCP-25 gel treated mice as compared to mice treated with the control gel (FIG. 17A). Furthermore, a reduction in bacterial bioluminescence was observed after treatment with TCP-25 gel as compared to treatment with control gel (FIG. 17B). The wound healing rate was significantly improved in TCP-25 gel treated mice as compared to control gel treated mice (FIG. 17C). In other words, treatment with TCP-25 gel resulted in improved wound healing. This is indicative of that TCP-25 treatment decreases the wound area over time.

### Example 4—Clinical Study Protocol

| Summary | |
|---|---|
| EudraCT Number: | 2021-004728-14 |
| Sponsor's Protocol Code Number: | TCP25-001 |
| National Competent Authority: | Sweden - MPA |
| Clinical Trial Type: | EEA CTA |
| Trial Status: | Ongoing |

Index
A. PROTOCOL INFORMATION
B. SPONSOR INFORMATION
C. APPLICANT IDENTIFICATION
D. IMP IDENTIFICATION
D. 8 INFORMATION ON PLACEBO
E. GENERAL INFORMATION ON THE TRIAL
F. POPULATION OF TRIAL SUBJECTS
G. INVESTIGATOR NETWORKS TO BE INVOLVED IN THE TRIAL
N. REVIEW BY THE COMPETENT AUTHORITY OR ETHICS
COMMITTEE IN THE COUNTRY CONCERNED
P. END OF TRIAL

A. Protocol
Information

| | | |
|---|---|---|
| A.1 | Member State | Sweden - MPA |
| | Concerned | |
| A.2 | EudraCT number | 2021-004728-14 |
| A.3 | Full title of the trial | A Three-part, Phase I Study to Evaluate Safety, |
| | | Tolerability, and Pharmacokinetics (PK) of |
| | | Topical Doses of TCP-25 in Healthy |
| | | Male and Female Volunteers with Epidermal |
| | | Suction Blister Wounds and in Patients with Non- |
| | | Healing Leg Ulcers and Patients |
| | | with Dystrophic Epidermolysis Bullosa (DEB) |
| A.3.1 | Title of the trial for | A Three-part, Phase I Study to Evaluate Safety, |
| | lay people, in | Tolerability, and Pharmacokinetics (PK) of |
| | easily understood, | Topical Doses of TCP-25 in Healthy |
| | i.e. | Male and Female Volunteers with Epidermal |
| | non-technical, | Suction Blister Wounds and in Patients with Non- |
| | language | Healing Leg Ulcers and Patients |
| | | with Dystrophic Epidermolysis Bullosa (DEB) |
| A.4.1 | Sponsor's protocol | TCP25-001 |
| | code number | |
| A.7 | Trial is part of a | No |
| | Paediatric | |
| | Investigation Plan | |
| A.8 | EMA Decision number | |
| | of Paediatric | |
| | Investigation Plan | |

B. Sponsor Information
B. Sponsor: 1

| | | |
|---|---|---|
| B.1.1 | Name of Sponsor | Xinnate AB |
| B.1.3.4 | Country | Sweden |
| B.3.1 and B.3.2 | Status of the sponsor | Commercial |
| B.4 Source(s) of | | |
| Monetary or Material | | |
| Support for the clinical | | |
| trial: | | |
| B.4.1 | Name of organisation | Xinnate AB |
| | providing support | |
| B.4.2 | Country | Sweden |
| B.5 Contact point | | |
| designated by the | | |
| sponsor for further | | |
| information on the trial | | |
| B.5.1 | Name of organisation | CTC Clinical Trial Consultants AB |
| B.5.2 | Functional name of | CRM |
| | contact point | |
| B.5.3 | Address: | |
| B.5.3.1 | Street Address | Dag Hammarskjöldsväg 10 B |
| B.5.3.2 | Town/city | Uppsala |
| B.5.3.3 | Post code | 75237 |
| B.5.3.4 | Country | Sweden |
| B.5.6 | E-mail | caroline.hammarstrom@ctc-ab.se |

D. IMP
Identification
D.IMP: 1

| | | |
|---|---|---|
| D.1.2 and D.1.3 | IMP Role | Test |
| D.2 | Status of the IMP to be used in the | |
| | clinical trial | |
| D.2.1 | IMP to be used in the trial has a | No |
| | marketing authorisation | |
| D.2.5 | The IMP has been designated in this | No |
| | indication as an | |
| | orphan drug in the Community | |
| D.2.5.1 | Orphan drug designation number | |
| D.3 Description | | |
| of the IMP | | |
| D.3.1 | Product name | TCP-25 |
| D.3.2 | Product code | TCP-25 |
| D.3.4 | Pharmaceutical form | Transdermal gel |
| D.3.4.1 | Specific paediatric formulation | No |
| D.3.7 | Routes of administration for this IMP | Topical |
| D.3.8 to | | |
| D.3.10 IMP | | |
| Identification | | |
| Details (Active | | |
| Substances) | | |
| D.3.8 | INN - Proposed INN | TCP-25 |
| D.3.9.2 | Current sponsor code | TCP-25 |
| D.3.9.3 | Other descriptive name | H-Gly-Lys-Tyr-Gly-Phe- |
| | | Tyr-Thr-His-Val-Phe- |
| | | Arg-Leu-Lys-Lys-Trp-Ile- |
| | | Gln-Lys-Val-Ile-Asp-Gln- |
| | | Phe-Gly-Glu-OH |
| D.3.9.4 | EV Substance Code | SUB245389 |
| D.3.10 | Strength | |
| D.3.10.1 | Concentration unit | mg/ml |
| | | milligram(s)/millilitre |
| D.3.10.2 | Concentration type | range |
| D.3.10.3 | Concentration number | 0.86 to 8.6 |
| D.3.11 The IMP | | |
| contains an: | | |
| D.3.11.1 | Active substance of chemical origin | Yes |
| D.3.11.2 | Active substance of biological/ | No |
| | biotechnological origin (other | |
| | than Advanced Therapy IMP (ATIMP) | |
| The IMP is a: | | |
| D.3.11.3 | Advanced Therapy IMP (ATIMP) | No |
| D.3.11.3.1 | Somatic cell therapy medicinal product | No |
| D.3.11.3.2 | Gene therapy medical product | No |
| D.3.11.3.3 | Tissue Engineered Product | No |
| D.3.11.3.4 | Combination ATIMP (i.e. one involving a | No |
| | medical device) | |
| D.3.11.3.5 | Committee on Advanced therapies (CAT) | No |
| | has issued a classification for this | |
| | product | |
| D.3.11.4 | Combination product that includes a | No |
| | device, but does not involve an | |
| | Advanced Therapy | |
| D.3.11.5 | Radiopharmaceutical medicinal product | No |
| D.3.11.6 | Immunological medicinal product (such | No |
| | as vaccine, allergen, immune serum) | |
| D.3.11.7 | Plasma derived medicinal product | No |
| D.3.11.8 | Extractive medicinal product | No |
| D.3.11.9 | Recombinant medicinal product | No |
| D.3.11.10 | Medicinal product containing genetically | No |
| | modified organisms | |
| D.3.11.11 | Herbal medicinal product | No |
| D.3.11.12 | Homeopathic medicinal product | No |
| D.3.11.13 | Another type of medicinal product | No |

D.8 Information
on Placebo
D.8 Placebo: 1

| | | |
|---|---|---|
| D.8.1 | Is a Placebo used in this Trial? | Yes |
| D.8.3 | Pharmaceutical form of the placebo | Transdermal |
| | | gel |
| D.8.4 | Route of administration of the placebo | Topical |

E. General
Information
on the Trial
E.1 Medical
condition or
disease under
investigation

| | | |
|---|---|---|
| E.1.1 | Medical | TCP-25 gel targets both bacteria and |
| | condition(s) being | inflammation, aspects common to many |
| | investigated | wounds and is being developed for a range of |
| | | wound healing indications including |
| | | prevention and treatment of infection and |
| | | inflammation in Epidermolysis bullosa wounds |
| | | and in other acute or non-healing wounds. |
| E.1.1.1 | Medical condition in | Treatment is to be given to patients with |
| | easily understood | Epidermolysis Bullosa to promote wound |
| | language | healing. |
| E.1.1.2 | Therapeutic area | Diseases [C] - Bacterial Infections and |
| | | Mycoses [C01] |
| MedDRA | | |
| Classification | | |
| E.1.2 Medical | | |
| condition or | | |
| disease under | | |
| investigation | | |
| E.1.2 Version | | |
| 20.0 | | |
| E.1.2 Level PT | | |
| E.1.2 | | |
| Classification | | |
| code 10014989 | | |
| E.1.2 Term | | |
| Epidermolysis | | |
| bullosa | | |
| E.1.2 System | | |
| Organ Class | | |
| 10010331 - | | |
| Congenital, | | |
| familial and | | |
| genetic disorders | | |
| E.1.3 | Condition being | Yes |
| | studied is a rare | |
| | disease | |
| E.2 Objective | | |
| of the trial | | |
| E.2.1 | Main objective of | Part 1: The primary objective is to evaluate |
| | the trial | the safety and tolerability of three dose levels |
| | | of TCP-25 gel (0.86 mg/mL, 2.9 |
| | | mg/ml and 8.6 mg/mL) applied topically to |
| | | epidermal suction blister wounds. |
| | | Part 2: The primary objective is to evaluate |
| | | the safety and tolerability of two doses of |
| | | TCP-25 gel (2.9 mg/mL and |
| | | 8.6 mg/mL) after applied topically to non- |
| | | healing leg ulcers. |
| | | Part 3: The primary objective is to evaluate |
| | | the safety and tolerability of up to four dose |
| | | levels of TCP-25 applied by two |
| | | strengths of TCP-25 gel (2.9 mg/mL and 8.6 mg/mL, |
| | | respectively) topically to patients with DEB. |
| E.2.2 | Secondary objectives | Part 1: The secondary objective is to evaluate |
| | of the trial | the systemic exposure of TCP-25 after applied |
| | | topically to epidermal suction blister wounds. |
| | | Part 2: The secondary objective is to evaluate |
| | | the systemic exposure of TCP-25 after applied |
| | | topically to non-healing leg ulcers. |
| | | Part 3: The secondary objective is to evaluate |
| | | the systemic exposure of TCP-25 when |
| | | applied topically in up to four dose levels |
| | | by two strengths of TCP-25 gel (2.9 mg/mL and |
| | | 8.6 mg/mL) topically to patients with DEB. |
| E.2.3 | Trial contains a | No |
| | sub-study | |
| E.3 | Principal inclusion | Part 1 |
| | criteria | 1. Willing and able to give written informed |
| | | consent for participation in the study |
| | | 2. Healthy male or female subject 18-60 years |
| | | (inclusive) of age at the time of signing the |
| | | informed consent. |
| | | 3. Body Mass Index (BMI) ≥18.0 and ≤30.0 kg/m2 |
| | | 4. Healthy and intact skin where the blister |
| | | suction wounds will be induced |
| | | 5. Women of childbearing potential (WOCBP) |
| | | must have a documented negative serum |
| | | pregnancy test done at the screening |
| | | visit, within 4 weeks prior to suction blister |
| | | formation and the start of study treatment. |
| | | WOCBP must practice abstinence |
| | | from at least 4 weeks prior to dose to 4 weeks |
| | | after last dose. Female subjects must refrain |
| | | from donating eggs from the |
| | | date of dosing until 3 months after dosing with |
| | | the IMP. Their male partner must agree to use |
| | | a condom during the same |
| | | time frame if he has not undergone |
| | | vasectomy. Male subjects must be willing to |
| | | use condom or be vasectomised or practice |
| | | sexual abstinence to prevent pregnancy and |
| | | drug exposure of a partner and refrain from |
| | | donating sperm from the date of |
| | | last dosing until 3 months after the last dosing |
| | | with the IMP. Their female partner of child- |
| | | bearing potential must use |
| | | contraceptive methods with a failure rate of <1% |
| | | to prevent pregnancy |
| | | 6. Clinically relevant medical history, physical |
| | | findings, vital signs, ECG and laboratory values |
| | | at the time of screening, as judged by the |
| | | Investigator |
| | | Part 2: |
| | | 1. Willing and able to give written informed |
| | | consent for participation in the study |
| | | 2. Male patient, or female patient of non- |
| | | childbearing potential, ≥40 years of age at the |
| | | time of signing the informed |
| | | consent. |
| | | 3. Male subjects must be willing to use |
| | | condom or be vasectomized or practice |
| | | sexual abstinence to prevent pregnancy and |
| | | drug exposure of a partner and refrain from |
| | | donating sperm from the date of last dosing |
| | | until 3 months after the last dosing |
| | | with the IMP. Their female partner of child- |
| | | bearing potential must use contraceptive |
| | | methods with a failure rate of <1% to |
| | | prevent pregnancy. |
| | | 4. Clinically relevant medical history, vital |
| | | signs, ECG and laboratory values at the time |
| | | of screening, as judged by the Investigator. |
| | | 5. Patients diagnosed with venous |
| | | insufficiency by relevant physiological tests |
| | | including doppler or venous plethysmography |
| | | or diagnosed by relevant clinical evaluations. |
| | | 6. Systolic index >0.6 (Data from medical |
| | | records from the last 2 years, or the |
| | | assessment should be repeated at the |
| | | screening visit) |
| | | 7. Ulcer duration >6 weeks |
| | | 8. Total target ulcer area applicable for |
| | | treatment: ≥40 and ≤120 cm2 (as measured |
| | | with the Silhouette imaging equipment |
| | | at the screening visit). (The target ulcer area |
| | | may not consist of >2 separate ulcers) |
| | | 9. Ability to tolerate compression bandaging |
| | | 10. Willing to attend study site visits |
| | | Part 3 |
| | | 1. Willing and able to give written informed |
| | | consent for participation in the study. For 15 to |
| | | 17-year-olds: A separate consent |
| | | is required from both (if applicable) the |
| | | patient's parents/legal guardians |
| | | 2. Male patient, or female patient of non- |
| | | childbearing potential with documented |
| | | diagnosis of inherited DEB, ≥15 years of age |
| | | at the time of signing the informed consent. |
| | | 3. Male subjects must be willing to use |
| | | condom or be vasectomized or practice |
| | | sexual abstinence to prevent pregnancy and |
| | | drug exposure of a partner and refrain from |
| | | donating sperm from the date of last dosing |
| | | until 3 months after the last dosing |
| | | with the IMP. Their female partner of child- |
| | | bearing potential must use contraceptive |
| | | methods with a failure rate of <1% to |
| | | prevent pregnancy |
| | | 4. Clinically relevant medical history at the time |
| | | of screening, as judged by the Investigator |
| | | 5. Presence of two target wound areas of 50 |
| | | cm2.The primary wound area should not be |
| | | located at anatomical sites with high |
| | | likelihood of accidental trauma (e.g., knee, |
| | | elbow). The secondary wound area could be |
| | | located at anatomical sites with high |
| | | likelihood of accidental trauma or be within the |
| | | higher age span. Both wound areas should |
| | | meet all the following characteristics: |
| | | a. Including open wound with a surface area of ≤30 |
| | | cm2 (as measured with the Silhouette imaging |
| | | equipment at the screening visit). |
| | | b. At Visit 2 (first IMP treatment), each target |
| | | wound should not present a surface reduction ≥30% |
| | | from the Screening visit |
| | | c. Wound aged ≥3 weeks to <9 months at the |
| | | Screening visit |
| | | 6. Presence of a reference wound area of 50 |
| | | cm2 to be treated with standard of care only |
| | | and to be included in the |
| | | exploratory assessment. The wound area |
| | | should match the primary wound area for |
| | | following characteristics: |
| | | a. Including open wound with a surface area of ≤30 |
| | | cm2 (as measured with the Silhouette imaging |
| | | equipment at the screening visit). |
| | | b. At Visit 2 (first IMP treatment), the wound |
| | | should not present a surface reduction ≥30% |
| | | from the Screening visit |
| | | c. Wound aged ≥3 weeks to <9 months at the |
| | | Screening visit |
| | | d. Not located at anatomical sites with high |
| | | likelihood of accidental trauma (e.g., knee, |
| | | elbow) |
| | | 7. Willing to attend study site visits |
| E.4 | Principal exclusion | Part 1: |
| | criteria | 1. History of any clinically significant disease or |
| | | disorder which, in the opinion of the |
| | | Investigator, may either put the subject |
| | | at risk because of participation in the study, or |
| | | influence the results or the subject's ability to |
| | | participate in the study. |
| | | 2. Disease that may interfere with wound |
| | | healing, e.g., diabetes type I/II, arterial-, |
| | | renal-, liver, or cardiac insufficiency, |
| | | chronic obstructive lung disease, cancer, |
| | | autoimmune disease, edema at the study site, |
| | | severe obesity, or previous known |
| | | wound healing problems, as judged by the |
| | | investigator. |
| | | 3. Active skin disease, e.g., dermatitis, |
| | | psoriasis and wounds, and/or tattoos in the |
| | | areas where suction blister wounds will be |
| | | induced, as judged by the investigator. |
| | | 4. Any planned major surgery within the |
| | | duration of the study. |
| | | 5. After 10 minutes supine rest at the time of |
| | | screening, any vital signs values outside the |
| | | following ranges: |
| | | Systolic blood pressure <90 or >160 mmHg, or |
| | | Diastolic blood pressure <50 or >100 mmHg, or |
| | | Pulse <40 or >90 beats per minute (bpm) |
| | | 6. Any clinically significant abnormalities in the |
| | | resting ECG at the time of screening, as |
| | | judged by the Investigator. |
| | | 7. Current smokers or users of nicotine |
| | | products. Irregular use of nicotine (e.g., |
| | | smoking, snuffing, chewing tobacco) less |
| | | than three times per week is allowed before |
| | | screening visit. |
| | | 8. Female subjects who are pregnant or |
| | | lactating or planning a pregnancy. |
| | | 9. Systemic immunosuppressive treatment. |
| | | 10. Subjects who are currently receiving or |
| | | have received the following treatments within |
| | | 2 weeks prior to screening are |
| | | excluded from the study: |
| | | systemic corticosteroids or |
| | | immunosuppressant agents; or |
| | | antibiotics via any route |
| | | 11. Regular use of anticoagulants (i.e., |
| | | heparin, warfarin, coumarins, other |
| | | anticoagulants per Investigator's judgement) or |
| | | non-steroidal anti-inflammatory drugs |
| | | (NSAIDs) within 2 weeks prior to the (first) |
| | | administration of IMP, at the discretion of |
| | | the Investigator. |
| | | 12. History of severe allergy/hypersensitivity or |
| | | ongoing allergy/hypersensitivity, as judged by |
| | | the Investigator, or history of |
| | | hypersensitivity to drugs with a similar |
| | | chemical structure or class to TCP-25 or to |
| | | any excipients of the hydrogel. |
| | | 14. History of alcohol abuse or excessive |
| | | intake of alcohol, as judged by the |
| | | Investigator. |
| | | 15. Presence or history of drug abuse, as |
| | | judged by the Investigator |
| | | Part 2: |
| | | 1. Presence or documented illness, which in |
| | | the Investigator's opinion may negatively |
| | | affect wound healing or interfere with |
| | | the study conduct. |
| | | 2. Target wound present for more than 5 years. |
| | | 3. Clinical signs of infection in or around the |
| | | wound in need of antibiotic treatment. |
| | | 4. Albumin <25 g/L or capillary Hb <90 g/L, or |
| | | HbA1c >70 mmol/mol at the time of the |
| | | screening visit. |
| | | 5. Any planned major surgery within the |
| | | duration of the study. |
| | | 6. Patients who are currently receiving or have |
| | | received the following treatments within 2 |
| | | weeks prior to screening are |
| | | excluded from the study: |
| | | systemic corticosteroids above 5 mg |
| | | prednisolone or equivalent, or |
| | | immunosuppressant agents; or |
| | | antibiotics via any route. |
| | | 7. Regular use of anticoagulants (i.e., heparin, |
| | | warfarin, coumarins, other anticoagulants |
| | | affecting APTT and/or PK/INR per |
| | | Investigator's judgement) within 2 weeks prior |
| | | to the (first) administration of IMP, at the |
| | | discretion of the Investigator. |
| | | 8. History of severe allergy/hypersensitivity or |
| | | ongoing allergy/hypersensitivity, as judged by |
| | | the Investigator, or history of |
| | | hypersensitivity to drugs with a similar |
| | | chemical structure or class to TCP-25 or to |
| | | any excipients of the hydrogel. |
| | | Part 3 |
| | | 1. Any subtype of EB other than DEB. |
| | | 2. EB index wounds that have infection in need |
| | | of systemic antibiotic treatment. |
| | | 3. Presence of or documented illness, which in |
| | | the Investigator's opinion may negatively |
| | | affect wound healing or interfere |
| | | with the study conduct. |
| | | 4. Subject has evidence of a systemic infection |
| | | or has used systemic antibiotics for EB-related |
| | | infections within 7 days before |
| | | the screening visit (Visit 1). |
| | | 5. Administration of systemic corticosteroids |
| | | within 30 days (>10 mg daily of prednisolone |
| | | or corresponding) or of topical |
| | | corticosteroids on target wound areas |
| | | (primary, secondary and reference wound |
| | | areas) within 14 days before the screening |
| | | visit (Visit 1). Corticosteroids for inhalation, |
| | | ophthalmic, or intranasal use are permitted. |
| | | 6. Subject has undergone stem cell transplant |
| | | or gene therapy for the treatment of EB with |
| | | an effect on target wounds. |
| | | 7. History of malignancy, including basal cell |
| | | carcinomas or squamous cell carcinomas in |
| | | the wound areas that will be |
| | | included in the study. |
| | | 8. History of severe allergy/hypersensitivity or |
| | | ongoing allergy/hypersensitivity, as judged by |
| | | the Investigator, or history of |
| | | hypersensitivity to drugs with a similar |
| | | chemical structure or class to TCP-25 or to |
| | | any excipients of the hydrogel. |
| | | 9. Planned treatment or treatment with another |
| | | investigational drug within 3 months prior to |
| | | Day -1. |
| E.5 | | |
| End points | | |
| E.5.1 | Primary end | Part 1 and 2: |
| | point(s) | Frequency, intensity and seriousness of |
| | | adverse events (AEs). |
| | | Local tolerability: Incidence of abnormal local |
| | | reactions as compared to expected wound |
| | | healing outcome, by direct |
| | | assessment by Investigator: |
| | | Skin and wound erythema (abnormal reaction noted). |
| | | Skin and wound oedema and swelling |
| | | (abnormal reaction noted). |
| | | Wound necrosis, crusting, and hemorrhage |
| | | (abnormal reactions noted). |
| | | Wound purulent discharge as sign of |
| | | excessive bacterial colonization and/or |
| | | infection. |
| | | Clinically significant changes from baseline in |
| | | electrocardiogram (ECG), vital signs |
| | | (systolic/diastolic blood pressure, pulse |
| | | rate), safety laboratory parameters |
| | | (haematology, clinical chemistry and |
| | | coagulation) and physical examinations. |
| | | Part 3 |
| | | Frequency, intensity and seriousness of |
| | | adverse events (AEs). |
| | | Local tolerability: Incidence of abnormal local |
| | | reactions as compared to baseline, by direct |
| | | assessment by Investigator: |
| | | Peri-wound and open wound erythema of |
| | | treated area (abnormal reaction noted) |
| | | Peri-wound and open wound oedema and |
| | | swelling of treated area (abnormal reaction noted) |
| | | Peri-wound and open wound scaling of |
| | | treated area (abnormal reaction noted) |
| | | Peri-wound and open wound |
| | | stinging/burning of treated area (abnormal |
| | | reaction noted) |
| E.5.1.1 | Timepoint(s) of | Through-out the study period |
| | evaluation of this | |
| | end point | |
| E.5.2 | Secondary end | Part 1: |
| | point(s) | TCP-25 plasma concentrations as measured |
| | | at Day 1, Day 2, Day 3 and Day 5. |
| | | Part 2: |
| | | TCP-25 plasma concentrations as measured |
| | | at Day 8, 12, 15, 19, 22, 26 29 (optional), and |
| | | 33 (optional). |
| | | Part 3: |
| | | TCP-25 plasma concentrations as measured |
| | | at Day 22 |
| E.5.2.1 | Timepoint(s) of | Through-out the study period |
| | evaluation of this | |
| | end point | |
| E.6 and E.7 | | |
| Scope of the | | |
| trial | | |
| E.6 | | |
| Scope of the | | |
| trial | | |
| E.6.1 | Diagnosis | No |
| E.6.2 | Prophylaxis | No |
| E.6.3 | Therapy | No |
| E.6.4 | Safety | Yes |
| E.6.5 | Efficacy | No |
| E.6.6 | Pharmacokinetic | Yes |
| E.6.7 | Pharmacodynamic | No |
| E.6.8 | Bioequivalence | No |
| E.6.9 | Dose response | No |
| E.6.10 | Pharmacogenetic | No |
| E.6.11 | Pharmacogenomic | No |
| E.6.12 | Pharmacoeconomic | No |
| E.6.13 | Others | No |
| E.7 Trial | | |
| type and phase | | |
| E.7.1 | Human | Yes |
| | pharmacology | |
| | (Phase I) | |
| E.7.1.1 | First administration | Yes |
| | to humans | |
| E.7.1.2 | Bioequivalence study | No |
| E.7.1.3 | Other | No |
| E.7.1.3.1 | Other trial type | |
| | description | |
| E.7.2 | Therapeutic | No |
| | exploratory (Phase II) | |
| E.7.3 | Therapeutic | No |
| | confirmatory | |
| | (Phase III) | |
| E.7.4 | Therapeutic use | No |
| | (Phase IV) | |
| E.8 Design | | |
| of the trial | | |
| E.8.1 | Controlled | Yes |
| E.8.1.1 | Randomised | Yes |
| E.8.1.2 | Open | Yes |
| E.8.1.3 | Single blind | No |
| E.8.1.4 | Double blind | Yes |
| E.8.1.5 | Parallel group | No |
| E.8.1.6 | Cross over | No |
| E.8.1.7 | Other | Yes |
| E.8.1.7.1 | Other trial design | Part 1 = controlled, randomised and double |
| | description | blind. Part 2 and 3 = open |
| E.8.2 | Comparator of | |
| | controlled trial | |
| E.8.2.1 | Other medicinal | No |
| | product(s) | |
| E.8.2.2 | Placebo | Yes |
| E.8.2.3 | Other | No |
| E.8.2.4 | Number of treatment | 5 |
| | arms in the trial | |
| E.8.3 | The trial involves | Yes |
| | single site in the | |
| | Member State concerned | |
| E.8.4 | The trial involves | No |
| | multiple sites in the | |
| | Member State concerned | |
| E.8.5 | The trial involves | No |
| | multiple Member States | |
| E.8.6 | Trial involving sites | |
| | outside the EEA | |
| E.8.6.1 | Trial being conducted | No |
| | both within and | |
| | outside the EEA | |
| E.8.6.2 | Trial being conducted | No |
| | completely outside | |
| | of the EEA | |
| E.8.7 | Trial has a data | No |
| | monitoring committee | |
| E.8.8 | Definition of the | The end of the study is defined as the date of |
| | end of the trial and | the last visit of the last subject in the study. |
| | justification where it | |
| | is not the last visit | |
| | of the last subject | |
| | undergoing the trial | |
| E.8.9 | Initial estimate of | |
| | the duration of the | |
| | trial | |
| E.8.9.1 | In the Member State | |
| | concerned years | |
| E.8.9.1 | In the Member State | |
| | concerned months | |
| E.8.9.1 | In the Member State | |
| | concerned days | |

F. Population
of Trial
Subjects
F.1 Age Range

| | | |
|---|---|---|
| F.1.1 | Trial has subjects under 18 | Yes |
| F.1.1 | Number of subjects for this age range: | 5 |
| F.1.1.1 | In Utero | No |
| F.1.1.2 | Preterm newborn infants (up to | No |
| | gestational age <37 weeks) | |
| F.1.1.3 | Newborns (0-27 days) | No |
| F.1.1.4 | Infants and toddlers (28 days-23 months) | No |
| F.1.1.5 | Children (2-11 years) | No |
| F.1.1.6 | Adolescents (12-17 years) | Yes |
| F.1.1.6.1 | Number of subjects for this age range: | 5 |
| F.1.2 | Adults (18-64 years) | Yes |
| F.1.2.1 | Number of subjects for this age range: | 30 |
| F.1.3 | Elderly (>=65 years) | No |
| F.2 Gender | | |
| F.2.1 | Female | Yes |
| F.2.2 | Male | Yes |
| F.3 Group of | | |
| trial subjects | | |
| F.3.1 | Healthy volunteers | Yes |
| F.3.2 | Patients | Yes |
| F.3.3 | Specific vulnerable populations | Yes |
| F.3.3.1 | Women of childbearing potential not | No |
| | using contraception | |
| F.3.3.2 | Women of child-bearing potential | Yes |
| | using contraception | |
| F.3.3.3 | Pregnant women | No |
| F.3.3.4 | Nursing women | No |
| F.3.3.5 | Emergency situation | No |
| F.3.3.6 | Subjects incapable of giving consent | No |
| | personally | |
| F.3.3.7 | Others | No |
| F.4 Planned | | |
| number of | | |
| subjects to be | | |
| included | | |
| F.4.1 | In the member state | 35 |
| F.5 | Plans for treatment or care after the | None |
| | subject has ended | |
| | the participation in the trial (if it is | |
| | different from the expected normal | |
| | treatment of that condition) | |

G. Investigator Networks to be involved in the Trial

N. Review by the
Competent Authority or
Ethics Committee in the
country concerned

| | | |
|---|---|---|
| N. | Competent Authority | Authorised |
| | Decision | |
| N. | Ethics Committee Opinion | Favourable |
| | of the trial application | |
| N. | Ethics Committee Opinion: | |
| | Reason(s) for unfavourable | |
| | opinion | |

| P. End of Trial | | |
|---|---|---|
| P. | End of Trial Status | Ongoing |

TCP-25 gel targets both bacteria and inflammation, aspects common to many wounds and is being developed for a range of wound healing indications including prevention and treatment of infection and inflammation in Epidermolysis bullosa wounds and in other acute or non-healing wounds.

Treatment is to be given to patients with Epidermolysis Bullosa to promote wound healing.

### Exploratory Objectives

To evaluate signs of wound healing and wound quality by independent evaluators from photographs.

To collect and store samples from dressings and wound surface for future exploratory research into changes in microbiological, molecular, and biochemical markers reflecting inflammation and/or infection.

### Example 5—Clinical Study of Patients with DEB

### Material and Methods

### Clinical Study

A clinical study was conducted in patients 15 years or older with confirmed Dystrophic Epidermolysis Bullosa (DEB), as described in Example 4. In each patient, according to the selection criteria in Example 4, a matching wound pair of chronic wounds (primary wound and reference wound) and a secondary more severe wound, located at anatomical sites with high likelihood of accidental trauma or within the higher age span, was identified. The wound areas were evaluated from baseline (Day 1) to Day 29. The study has enrolled four participants who have completed the study.

### Treatment of Wound Areas (Primary Wound, Secondary Wound and Reference Wound)

The three wound areas of 50 cm² each were handled and cleaned according to standard of care. A sterile non-adhesive dressing, limited to polyurethane wound dressing (such as Mepilex Lite), was used to cover the open wound and adjacent skin area. After dressing removal, the wound areas were gently cleaned according to established routines for EB wounds. Solutions such as potassium permanganate, or other antiseptics applied locally were to be avoided during the study weeks on the study wound areas. However, hypochlorite or potassium permanganate baths were allowed if part of the regular cleaning routine.

Primary and Secondary wound areas; TCP-25 gel was applied at each wound dressing change, every 2^{nd} to 3^{rd} day. The sequential treatment periods consisting of two weeks with 2.9 mg/mL TCP-25 (0.05 mL/cm² TCP-25 gel per treatment per application during week 1, 0.1 mL/cm² TCP-25 gel per application during week 2) followed by two weeks of 8.6 mg/mL TCP-25 (0.05 mL/cm² TCP-25 gel per application during week 1, 0.1 mL/cm² TCP-25 gel per application during week 2). In total, each patient received 12 dose applications (6 applications TCP-25 2.9 mg/mL+6 applications TCP-25 8.6 mg/mL) during the study (Days 1, 3, 6, 8, 10, 13, 15, 17, 20, 22, 24 and 27). The gel was applied from vials using either a plastic syringe or spatula. For the 0.05 mL/cm² dose volume, the content of one vial corresponds to a treatment area of 50 cm². For the 0.1 mL/cm² dose volume, the content of two vials corresponds to a treatment area of 50 cm².

The reference wounds were treated with standard of care.

The primary wound is also referred to herein as “simple wound”. The secondary wound is also referred to herein as “complex wound”. For the purpose of biomarker assessments, additional larger reference (non-TCP-treated) wounds were identified in 2 patients to match the larger secondary wounds.

### TCP-25 Gels

The composition of the gel is shown below.

Middle strength: 2.9 mg/mL TCP-25, 1.2% Natrosol 250 HX, 2% Glycerol, in 10 mM Tris pH 7.

High strength: 8.6 mg/mL TCP-25, 1% Natrosol 250 HX, 2% Glycerol, in 10 mM Tris pH 7.

### Measuring Wound Area and Calculation of Wound Healing

Photos for assessment of signs of wound healing and wound quality, specifically the open wound area was measured using Silhouette wound camera system.

### Measuring Wound Leakage, HPB and Cytokines

Scoring of leakage in dressings was performed using a visual scoring scale from 0-5, where 0 represented “No exudation in wound area” and 5 represented “exudation soaking the dressing in an area exceeding the wound area”.

Samples from dressings were collected from each of the wounds for determining the amount of heparin-binding protein (HBP) and cytokines according to the following procedure:

At each dressing change, the dressing was removed from the wound, placed into an empty sterile petri dish, and kept on ice. The dressing and 50 milliliters of cold, sterile 10 mM Tris buffer at pH 7.4 were added into a 60 ml syringe and then vortexed. The fluid was then extracted from the dressing by squeezing the dressing in the syringe. The extracted fluid was kept on ice. We added 100× Halt Protease Inhibitor Cocktail (Thermo Fisher Scientific, USA) to half of the dressing fluid per the manufacturer's directions to a final strength of 1×, and samples were then aliquoted and frozen at −80° C.

Cytokines in the dressing fluid were quantified in the stored dressing fluid samples that contained protease inhibitor. Cytokines were measured using a V-PLEX Proinflammatory Panel (human) kit (Meso Scale Diagnostics, Rockville, MD, USA, art. no: K15049D) per the manufacturer's instructions. The concentration of each cytokine was calculated using the MSD discovery Workbench analysis software. The cytokine levels were expressed as concentration per cm² wound area.

The amount of heparin-binding protein (HBP) was determined using an ELISA kit from Aviva Systems Biology (San Diego, CA, USA). Samples were analyzed according to the manufacturer's instructions in duplicate. ELISA signals were read at 450 nm on a microplate reader (BioRad, Hercules, CA, USA). A standard curve was established with reference standards to calculate the concentration in each sample.

### Results

### Wound Area

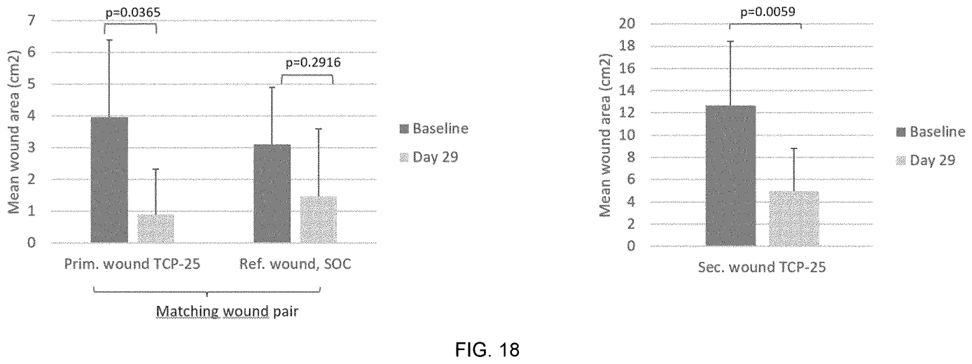

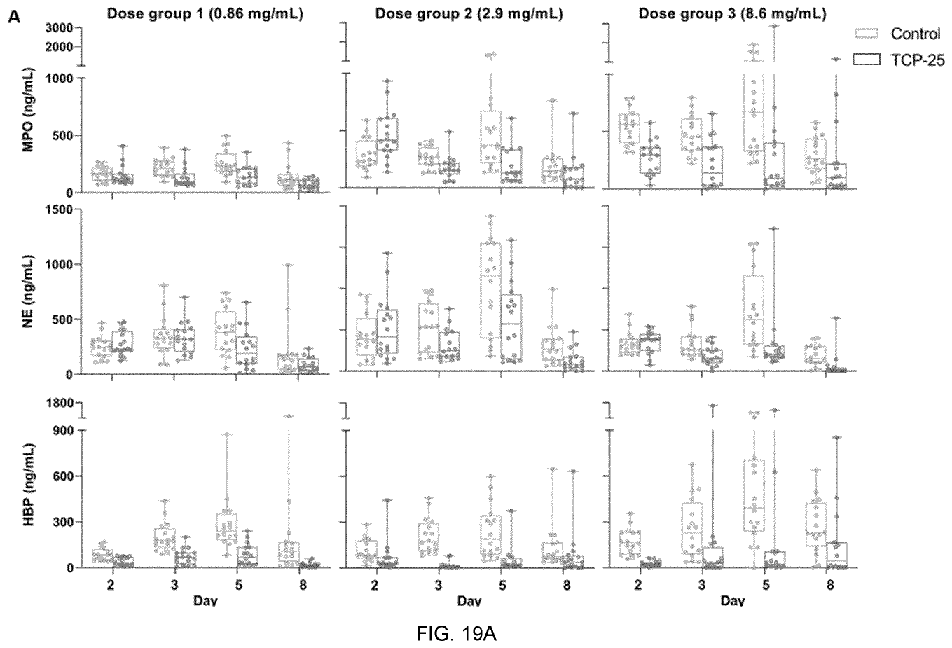

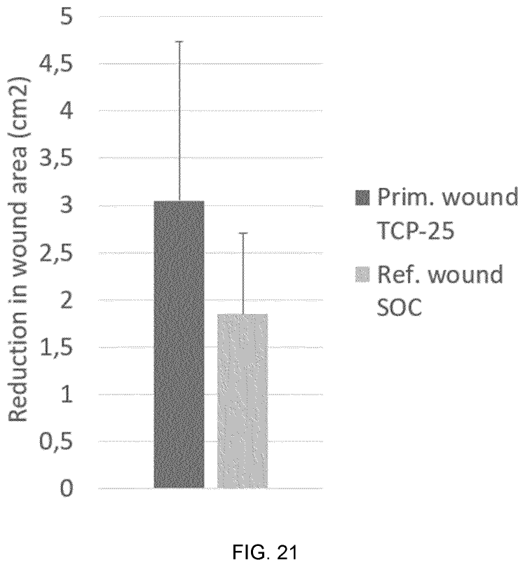

The results of the study show a significant reduction in wound sizes for patients with DEB over a treatment period of 29 days (FIG. 18, FIG. 21 and table below), both for the primary wound and the secondary wound. The latter located in an anatomical more challenging sites, assessed as harder to heal and treat. The application of TCP-25, at different concentrations, to these chronic, non-healing DEB wounds in four patients yielded notable effects, as observed and quantified through measuring the wound area, demonstrating effectiveness of the TCP treatment and healing process for DEB patients.

Importantly, the healing rate was significantly higher than reported for chronic DEB wounds of the type included.

| | Primary | | Secondary |
|---|---|---|---|
| | wound | Reference wound, | wound, |
| | TCP-25 | standard of care | TCP-25 |
| Baseline (cm²) | 4.0 | 3.1 | 12.7 |
| Day 29 (cm²) | 0.9 | 1.5 | 5.0 |
| Reduction at open area | 3.1 | 1.6 | 7.7 |
| from baseline (cm²) | | | |

### Wound Leakage, HPB and Cytokines

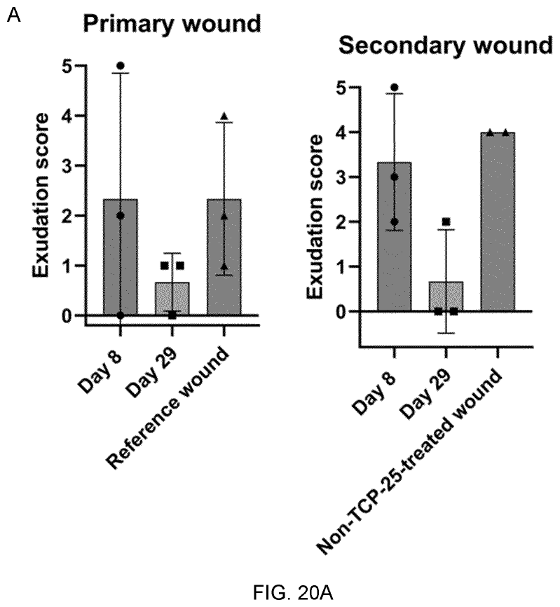

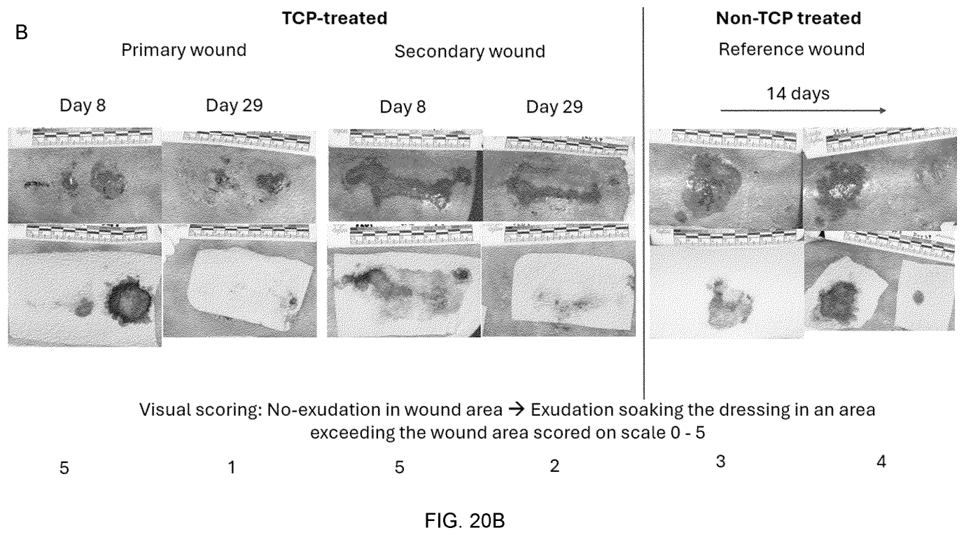

TCP-25 treatment reduced wound leakage (i.e. wound exudation) in DEB wounds. Leakage was reduced after treatment with TCP-25 gel at day 29 relative to day 8. The leakage after 4 weeks of treatment (day 29) was lower than observed for non-TCP-25 treated wounds. FIG. 20A shows average wound leakage of all primary (i.e “simple”) TCP-25 treated wounds (n=3) at day 8 and 29 and for reference SOC wounds (n=3). It further shows average wound leakage of secondary (i.e. “complex”) TCP-25 treated wounds (n=3) at day 8 and day 29 compared to additional non-TCP treated wounds of similar size (n=2). FIG. 20B illustrates wound leakage of primary, secondary and reference SOC wounds for one of the treated patients.

Wound fluid from wounds treated with TCP-25 for 29 days showed reduces HBP levels when compared with reference non-TCP-25-treated wounds. The amount of heparin binding protein (HBP) in the wound fluid from complex wounds of two patients is shown in the table below.

| | After 29 days of treatment: | |
|---|---|---|
| | TCP-treated secondary | Non-TCP-treated |
| | wound | wound (SOC) |
| Wound pair 1 | 582 ng/ml | 2005 ng/ml |
| Wound pair 2 | 421 ng/ml | 2154 ng/ml |

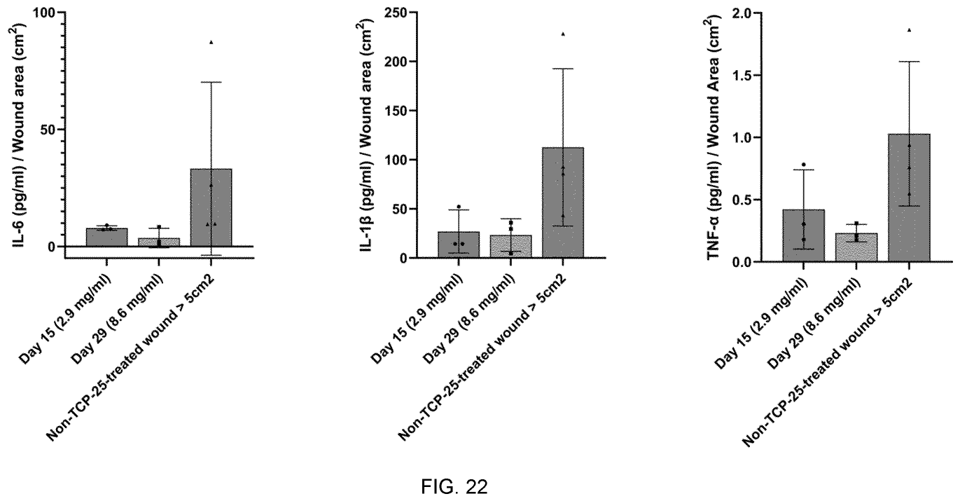

A reduction of IL-1β, TNF-α, and IL-6 levels in the secondary TCP-treated wounds was observed after treatment for 15 and 29 days compared with non-TCP25-treated wounds of similar size, larger than 5 cm² (FIG. 22).

TABLE
Mean and standard deviation (SD) of cytokine
concentrations (pg/ml)/Wound area (cm²).

| | IL-6 | IL-1b | TNF-a |
|---|---|---|---|
| Secondary Wound Day 15 | 7.882, | 27.02, | 0.4212, |
| (2.9 mg/ml) | SD 0.9997 | SD 21.90 | SD 0.3187 |
| Secondary Wound Day 29 | 3.610, | 23.33, | 0.2313, |
| (8.6 mg/ml) | SD 4.152 | SD 16.59 | SD 0.07046 |
| Non-TCP-25-treated | 33.22, | 112.6, | 1.029, |
| wounds (SOC) | SD 36.94 | SD 80.07 | SD 0.5799 |

In patients with recessive DEB in particular, continuous blistering, inflammation, relapsing infections and disturbed regeneration lead to painful, chronically inflamed often non-healing wounds. Additionally, repeated minor mechanical stress before the wound-healing process is complete limits the potential for cutaneous regeneration. Consequently, over time, these chronic wounds are accompanied by fibrosis, scarring, mitten deformities and, frequently, a deadly aggressive form of metastatic squamous cell carcinoma.

Recently, a number of investigations have focused on the immunological aspects of EB, and accumulating studies now support that immunological mechanisms promote the wound development. In line with this, studies have also provided evidence for an intrinsic pro-inflammatory state in EB patients. For example, high levels of cytokines, such as interleukin (IL)-1β and IL-6, have been observed. The high proinflammatory state not only hampers healing, but also drives both fibrosis and skin carcinoma development. Thus, these results indicate that TCP-25 mediated reduction of cytokines may improve wound healing and reduce the risk for fibrosis and cancer development.

Sequence overview

| SEQ ID NO | Sequence |
|---|---|
| 1 | GKYGFYTHVFRLKKWIQKVIDQFGE |
| 2 | FYTHVFRLKKWIQKVIDQFGE |
| 3 | GKYGFYTHVFRLKKWIQKVI |
| 4 | HVFRLKKWIQKVIDQFGE |
| 5 | KYGFYTHVFRLKKWIQKVIDQFGE |
| 6 | GKYGFYTHVFRLKKWIQKVIDQF |
| 7 | GKYGFYTHVFRLKKWIQKV |
| 8 | KKWIQKVIDQFGE |

### REFERENCES

Papanikolaou, M., A. Onoufriadis, J. E. Mellerio, L. A. Nattkemper, G. Yosipovitch, M. Steinhoff, and J. A. McGrath. 2021. ‘Prevalence, pathophysiology and management of itch in epidermolysis bullosa’, Br J Dermatol, 184:816-25.
Bernasconi, R., K. Thriene, E. Romero-Fernandez, C. Gretzmeier, T. Kuhl, M. Maler, P. Nauroy, S. Kleiser, A. C. Ruhl-Muth, M. Stumpe, D. Kiritsi, S. F. Martin, B. Hinz, L. Bruckner-Tuderman, J. Dengjel, and A. Nystrom. 2021. ‘Pro-inflammatory immunity supports fibrosis advancement in epidermolysis bullosa: intervention with Ang-(1-7)’, EMBO Mol Med, 13: e14392.
van der Kooi-Pol, M. M., Y. K. Veenstra-Kyuchukova, J. C. Duipmans, G. N. Pluister, L. M. Schouls, A. J. de Neeling, H. Grundmann, M. F. Jonkman, and J. M. van Dijl. 2012. ‘High genetic diversity of Staphylococcus aureus strains colonizing patients with epidermolysis bullosa’, Exp Dermatol, 21:463-6.
Hoste, E., E. N. Arwert, R. Lal, A. P. South, J. C. Salas-Alanis, D. F. Murrell, G. Donati, and F. M. Watt. 2015. ‘Innate sensing of microbial products promotes wound-induced skin cancer’, Nat Commun, 6:5932.
Robertson, S. J., E. Orrin, M. K. Lakhan, G. O'Sullivan, J. Felton, A. Robson, D. T. Greenblatt, C. Bernardis, J. A. McGrath, A. E. Martinez, and J. E. Mellerio. 2021. ‘Cutaneous Squamous Cell Carcinoma in Epidermolysis Bullosa: a 28-year Retrospective Study’, Acta Derm Venereol, 101: adv00523.
Tang, J. Y., M. P. Marinkovich, E. Lucas, E. Gorell, A. Chiou, Y. Lu, J. Gillon, D. Patel, and D. Rudin. 2021. ‘A systematic literature review of the disease burden in patients with recessive dystrophic epidermolysis bullosa’, Orphanet J Rare Dis, 16:175.
Cutting, Br J Comm Nurse, 2003
Adderley, Wound essentials, vol 3, 2008
Pillay and Clapham, 2018, Wounds international, vol 9, 4
Pope et al., Am Acad Dermatol. 2012 November
Prodinger, C., J. Reichelt, J. W. Bauer, and M. Laimer. 2019. ‘Epidermolysis bullosa: Advances in research and treatment’, Exp Dermatol, 28:1176-89.
Levin et al. 2021. ‘Characterization of wound microbes in epidermolysis bullosa: Results from the epidermolysis bullosa clinical characterization and outcomes database’, Pediatric Dermatology, 38:119-125.
Saravanan, R., D. A. Holdbrook, J. Petrlova, S. Singh, N. A. Berglund, Y. K. Choong, S. Kjellstrom, P. J. Bond, M. Malmsten, and A. Schmidtchen. 2018. ‘Structural basis for endotoxin neutralisation and anti-inflammatory activity of thrombin-derived C-terminal peptides’, Nat Commun, 9:2762
van der Plas, M. J., R. K. Bhongir, S. Kjellstrom, H. Siller, G. Kasetty, M. Morgelin, and A. Schmidtchen. 2016. ‘Pseudomonas aeruginosa elastase cleaves a C-terminal peptide from human thrombin that inhibits host inflammatory responses’, Nat Commun, 7:11567.
Kalle, M., P. Papareddy, G. Kasetty, M. Morgelin, M. J. van der Plas, V. Rydengard, M. Malmsten, B. Albiger, and A. Schmidtchen. 2012. ‘Host defense peptides of thrombin modulate inflammation and coagulation in endotoxin-mediated shock and Pseudomonas aeruginosa sepsis’, PLOS One, 7: e51313.
Puthia, M., M. Butrym, J. Petrlova, A. C. Stromdahl, M. A. Andersson, S. Kjellstrom, and A. Schmidtchen. 2020. ‘A dual-action peptide-containing hydrogel targets wound infection and inflammation’, Sci Transl Med, 12.
Ahlstrom, M. G., L. M. R. Gjerdrum, H. F. Larsen, C. Fuchs, A. L. Sorensen, J. L. Forman, M. S. Agren, and M. Mogensen. 2018. ‘Suction blister lesions and epithelialization monitored by optical coherence tomography’, Skin Res Technol, 24:65-72.
Bar et al., Clinical and Experimental Dermatology (2021) 46, pp 1223-1229
Byrd, Nature Reviews Microbiology, 2018, 16, 143
Bubier J A, et al. (2010) A Mouse Model of Generalized Non-Herlitz Junctional Epidermolysis Bullosa. J. Invest Dermatol 130 (7): 1819-28
Sproule et al. (2014) Molecular identification of collagen 17a1 as a major genetic modifier of laminin gamma 2 mutation-induced junctional epidermolysis bullosa in mice. PLOS Genet 10 (2): e1004068
Masson-Meyers D S, Andrade T A M, Caetano G F, Guimaraes F R, Leite M N, Leite S N, Frade M A C. Experimental models and methods for cutaneous wound healing assessment. Int J Exp Pathol. 2020 February; 101 (1-2): 21-37.

1. A compound for use in the treatment of a skin disease or skin condition in an individual in need thereof, wherein said skin disease or skin condition is selected from the group consisting of a disease or condition characterized by blistering, a disease or condition characterized by excessive wound exudate, a disease or condition characterised by a dysregulated skin microbiome and scars,
wherein the compound comprises a peptide comprising or consisting of the amino acid sequence
X1-X2-X3-X4-X5-X6-W-X8-X9-X10,
wherein
X_{4, 6, 9} is any standard amino acid,
X₁ is I, L or V,
X₂ is any standard amino acid except C,
X₃ is A, E, Q, R or Y,
X₅ is any standard amino acid except R,
X₈ is I or L,
X₁₀ is any standard amino acid except H,
wherein said peptide has a length of from 10 to 100 amino acid residues.

2. A compound for use in reducing the amount of fluid that exudes from one or more wound or blister in an individual in need thereof,
wherein the compound comprises a peptide comprising or consisting of the amino acid sequence
X1-X2-X3-X4-X5-X6-W-X8-X9-X10,
wherein
X_{4, 6, 9} is any standard amino acid,
X₁ is I, L or V,
X₂ is any standard amino acid except C,
X₃ is A, E, Q, R or Y,
X₅ is any standard amino acid except R,
X₈ is I or L,
X₁₀ is any standard amino acid except H,
wherein said peptide has a length of from 10 to 100 amino acid residues,
wherein the individual suffers from a disease or condition characterized by excessive wound exudate.

3. A compound for use in promoting a healthy skin microbiome in an individual in need thereof,
wherein the compound comprises a peptide comprising or consisting of the amino acid sequence
X1-X2-X3-X4-X5-X6-W-X8-X9-X10,
wherein
X_{4, 6, 9} is any standard amino acid,
X₁ is I, L or V,
X₂ is any standard amino acid except C,
X₃ is A, E, Q, R or Y,
X₅ is any standard amino acid except R,
X₈ is I or L,
X₁₀ is any standard amino acid except H,
wherein said peptide has a length of from 10 to 100 amino acid residues,
wherein the individual suffers from a disease or condition characterized by a dysregulated skin microbiome.

4. A compound for use in reducing scarring in an individual in need thereof,
wherein the compound comprises a peptide comprising or consisting of the amino acid sequence
X1-X2-X3-X4-X5-X6-W-X8-X9-X10,
wherein
X_{4, 6, 9} is any standard amino acid,
X₁ is I, L or V,
X₂ is any standard amino acid except C,
X₃ is A, E, Q, R or Y,
X₅ is any standard amino acid except R,
X₈ is I or L,
X₁₀ is any standard amino acid except H,
wherein said peptide has a length of from 10 to 100 amino acid residues,
wherein the individual suffers from a disease or condition characterized scarring.

5. The compound for use according to any one of the preceding claims, wherein said skin disease or skin disorder or said disease or condition is selected from the group consisting of:
epidermolysis bullosa (EB), burn-induced blisters, chilblains, cryotherapy-induced blisters, virus-induced blisters, chickenpox, shingles, herpes simplex, atypical enterovirus infection, hand, foot and mouth disease, eczema herpeticum, bullous impetigo and staphylococcal scalded skin syndrome, pemphigus, pemphigus foliaceous, pemphigus vulgaris, paraneoplastic pemphigus, pemphigus erythematosus, pemphigus vegetans,
pemphigoid, bullous pemphigoid, cicatricial pemphigoid, 200 k pemphigoid, mucous membrane pemphigoid, linear IgA bullous dermatosis, dermatitis herpetiformis, Porphyria cutanea tarda, bullous lupus erythematosus, Hailey-Hailey disease, Steven Johnson syndrome (toxic epidermal necrolysis),

6. The compound for use according to any one of the preceding claims, wherein said skin disease or skin disorder or said disease or condition is EB.

7. The compound for use according to any one of the preceding claims, wherein the skin disease or skin disorder or said disease or condition is EB, wherein EB is epidermolysis bullosa simplex, junctional epidermolysis bullosa, dystrophic epidermolysis bullosa, dominant dystrophic epidermolysis bullosa, recessive dystrophic epidermolysis bullosa, Kindler syndrome or epidermolysis bullosa acquisita.

8. The compound for use according to any one of the preceding claims, wherein said disease or condition is selected from the group consisting of burn wounds, chronic skin ulcers, infected acute wounds, pressure ulcers, chronic wounds, venous ulcers, venous leg ulcers, diabetic leg ulcers, eczema and atopic dermatitis,
wherein said disease or condition is characterized by blistering, excessive wound exudate, a dysregulated skin microbiome or scarring.

9. The compound for use according to any one of the preceding claims, wherein the compound is administered topically in a therapeutically effective amount.

10. The compound for use according to any one of the preceding claims, wherein the compound is administered to one or more areas of the individual affected by the skin condition or skin disorder.

11. The compound for use according to any one of the preceding claims, wherein treatment:
a. reduces the size of one or more blisters or wounds,
b. reduces the amount of fluid that exudes from one or more blister or wound,
c. reduces the size of the scar or reduces scar formation
d. promotes a healthy skin microbiome, and/or
e. reduces the total amount of microorganisms present in one or more wound or blister.

12. The compound for use according to any one of the preceding claims, wherein:
a. the peptide comprises or consists of the amino acid sequence:
X1-X2-X3-X4-X5-X6-W-X8-X9-X10-X11-X12-X13,
wherein
X4, 6, 9, 11 is any standard amino acid,
X1, is I, L or V
X2 is any standard amino acid except C
X3 is A, E, Q, R or Y
X5 is any standard amino acid except R
X8 is I or L
X10 is any standard amino acid except H
X12 is I, M or T
X13 is D, K, Q or R
and wherein said peptide has a length of from 20 to 100 amino acid residues;
or
b. the peptide comprises or consists of the amino acid sequence:
X1-X2-X3-X4-X5-X6-W-X8-X9-X10-X11-X12-X13-
X14-X15-X16-X17,
wherein
X4, 6, 9, 11, 14, 15 is any standard amino acid
X1 is I, L or V
X2 is any standard amino acid except C
X3 is A, E, Q, R or Y
X5 is any standard amino acid except R
X8 is I or L
X10 is any standard amino acid except H
X12 is I, M or T
X13 is D, K, Q or R
X16 is G or D
X17 is E, L, G, R or K
and wherein said peptide has a length of from 20 to 100 amino acid residues.

13. The compound for use according to any one of the preceding claims,
wherein the peptide has at least 80% sequence identity, such as at least 85% sequence identity, such as at least 90% sequence identity, such as at least 95% sequence identity with any of the amino acid sequences:
(SEQ ID NO: 1)
GKYGFYTHVFRLKKWIQKVIDQFGE
(SEQ ID NO: 2)
FYTHVFRLKKWIQKVIDQFGE,
(SEQ ID NO: 3)
GKYGFYTHVFRLKKWIQKVI,
(SEQ ID NO: 4)
HVFRLKKWIQKVIDQFGE,
(SEQ ID NO: 5)
KYGFYTHVFRLKKWIQKVIDQFGE,
(SEQ ID NO: 6)
GKYGFYTHVFRLKKWIQKVIDQF,
(SEQ ID NO: 7)
GKYGFYTHVFRLKKWIQKV,
or
(SEQ ID NO: 8)
KKWIQKVIDQFGE;
or
wherein the peptide comprises or consists of any of the amino acid sequences:
(SEQ ID NO: 1)
GKYGFYTHVFRLKKWIQKVIDQFGE,
(SEQ ID NO: 2)
FYTHVFRLKKWIQKVIDQFGE,
(SEQ ID NO: 3)
GKYGFYTHVFRLKKWIQKVI,
(SEQ ID NO: 4)
HVFRLKKWIQKVIDQFGE,
(SEQ ID NO: 5)
KYGFYTHVFRLKKWIQKVIDQFGE,
(SEQ ID NO: 6)
GKYGFYTHVFRLKKWIQKVIDQF,
(SEQ ID NO: 7)
GKYGFYTHVFRLKKWIQKV,
or
(SEQ ID NO: 8)
KKWIQKVIDQFGE;
or
wherein the peptide consists of the amino acid sequence:
(SEQ ID NO: 1)
GKYGFYTHVFRLKKWIQKVIDQFGE.

14. The compound for use according to any one of the preceding claims, wherein one or more of the standard amino acids comprised in the peptide are modified or derivatised.

15. The compound for use according to any one of the preceding claims, wherein one or more of the standard amino acids comprised in the peptide are PEGylated, amidated, acylated, acetylated, alkenylated and/or alkylated.

16. A composition comprising the compound for use according to any one of the preceding claims.

17. The composition according to claim 16, wherein the composition is an ointment, a cream, an emulsion, a lotion, a powder, a spray, a solution, a viscous solution, a gel or a hydrogel.

18. The composition according to any one of claims 16 to 17, wherein the composition is comprised in a patch, a dry patch, a plaster, a bandage, a film or a dressing.

19. The composition according to any one of claims 16 to 18, wherein the concentration of the peptide or compound in said composition is between 0.5 and 50 mg/mL, such as between 0.8 and 10 mg/mL, such as between 0.8 and 3 mg/mL, such as between 2.5 and 9 mg/mL, such as between 0.86 and 8.6 mg/mL, such as between 2.9 and 8.6 mg/mL.


SEQUENCE LISTING (8 sequences)

SEQ ID NO: 1 - protein, 25 aa, synthetic construct
GKYGFYTHVFRLKKWIQKVIDQFGE

SEQ ID NO: 2 - protein, 21 aa, synthetic construct
FYTHVFRLKKWIQKVIDQFGE

SEQ ID NO: 3 - protein, 20 aa, synthetic construct
GKYGFYTHVFRLKKWIQKVI

SEQ ID NO: 4 - protein, 18 aa, synthetic construct
HVFRLKKWIQKVIDQFGE

SEQ ID NO: 5 - protein, 24 aa, synthetic construct
KYGFYTHVFRLKKWIQKVIDQFGE

SEQ ID NO: 6 - protein, 23 aa, synthetic construct
GKYGFYTHVFRLKKWIQKVIDQF

SEQ ID NO: 7 - protein, 19 aa, synthetic construct
GKYGFYTHVFRLKKWIQKV

SEQ ID NO: 8 - protein, 13 aa, synthetic construct
KKWIQKVIDQFGE